(19) United States
(12) Patent Application Publication
(10) Pub. No.: US 2025/0075223 A1
(43) Pub. Date: Mar. 6, 2025

(54) COMPOSITIONS AND METHODS FOR GENOME EDITING ACROSS GRAFT-JUNCTIONS

(71) Applicants: The Texas A&M University System, College Station, TX (US); Washington State University, Pullman, WA (US)

(72) Inventors: Amit Dhingra, College Station, TX (US); Fabiola Ramirez Torres, Pullman, WA (US); Rishikesh Ghogare, Pullman, WA (US)

(21) Appl. No.: 18/816,382

(22) Filed: Aug. 27, 2024

Related U.S. Application Data
(60) Provisional application No. 63/579,179, filed on Aug. 28, 2023.

(51) Int. Cl. C12N 15/82; C12N 9/22

(52) U.S. Cl. CPC C12N 15/8201; C12N 9/22; C12N 2310/20; C12N 2770/00043

(57) ABSTRACT

Provided are compositions and methods for modifying a plant genome. Methods and compositions are also provided for delivering genome editing components across graft junctions and generating genome modified plants in the presence of a selection agent without integration of a selectable marker gene into the genome. Recombinant DNA constructs for modifying a plant genome and plants comprising the same are further provided.

### CROSS-REFERENCE TO RELATED APPLICATIONS

This application claims the benefit of U.S. provisional application No. 63/579,179, filed Aug. 28, 2023, herein incorporated by reference in its entirety.

### STATEMENT REGARDING FEDERALLY SPONSORED RESEARCH OR DEVELOPMENT

This invention was made with government support under grant NI19HFPXXXXXG010 awarded by United States Department of Agriculture through the National Institute of Food & Agriculture. The government has certain rights in the invention.

### INCORPORATION OF SEQUENCE LISTING

A sequence listing containing the file named “TAMC079US_ST26” which is 145 kilobytes (measured in MS-Windows®) and created on Aug. 2, 2024, and comprises 46 sequences, is incorporated herein by reference in its entirety.

### FIELD OF THE INVENTION

The present disclosure relates to the field of agricultural biotechnology, and to methods and compositions for delivery of genome editing components to plant cells across graft junctions. In particular, the invention relates to methods and compositions for producing stable gene-edited plants.

### BACKGROUND OF THE INVENTION

Precise genome editing technologies are powerful tools for engineering gene expression and modulating protein function and have the potential to improve important agricultural traits. A continuing need exists in the art to develop novel compositions and methods to effectively deliver genome editing components to generate edited crops with improved agronomic characteristics without the need for transgene elimination.

### SUMMARY

In one aspect, provided herein is a method for modifying a plant genome, the method comprising: obtaining a transgenic plant comprising a recombinant DNA construct encoding a site-specific endonuclease operably linked to a viral protein, or a rootstock therefrom; grafting a scion to said transgenic plant or rootstock therefrom, wherein the scion lacks said recombinant DNA construct; and selecting at least a first cell from said scion comprising a genomic modification resulting from the presence of said site-specific endonuclease. In one embodiment, the method further comprises generating a plant comprising the genomic modification from at least the first cell from said scion. In some embodiments, generating occurs in the presence of a selection agent; or by organogenesis or somatic embryogenesis. In further embodiments, organogenesis produces a root, a shoot, or somatic embryo comprising the genomic modification. In other embodiments, the recombinant DNA construct further comprises a sequence encoding a plant selectable marker. In other embodiments, the recombinant DNA construct is stably transformed into the genome of the transgenic plant; or further comprises a sequence capable of expressing at least one guide RNA sequence (gRNA).

In some embodiments, the transgenic plant further comprises a second recombinant DNA construct encoding: at least one viral replication protein; a movement protein; or a combination thereof. In particular embodiments, the second recombinant DNA construct encodes a viral protein from Tobacco Rattle Virus (TRV), Cabbage Leaf Curl Virus (CaLCuV), Bean Yellow Dwarf Virus (BcYDV), Pea early browning virus (PEBV), Tomato Mottle Mosaic Virus (TMMV), Bean Golden Mosaic Virus (BGMV), Tomato golden mosaic virus (TGMV), Cucumber Mosaic virus (CMV); or comprises a sequence encoding a protein having at least 85%, 90%, 91%, 92%, 93%, 94%, 95%, 96%, 97%, 98%, or 99% identity to a polypeptide selected from the group consisting of SEQ ID NO:43, SEQ ID NO:44, and SEQ ID NO:45.

In still other embodiments, the recombinant DNA construct encodes a viral protein from Tobacco Rattle Virus (TRV), Bean Yellow Dwarf Virus (BeYDV), Wheat Dwarf Virus (WDV), Tomato Mosaic Virus (ToMV), Pea Browning Virus (PEBV), Sonchus Yellow Net Rhabdovirus (SYNV), Potato Virus X (PVX), Foxtail Mosaic Virus (FoMV), Barley Yellow Striate Mosaic Virus (BYSMV), Beet Necrotic Yellow Vein Virus (BNYVV). In particular embodiments, the genomic modification is selected from the group consisting of a substitution, an insertion, an inversion, a deletion, a duplication, a transposition, and a combination thereof; or comprises introducing a heterologous DNA molecule into the plant genome. In further embodiments, the heterologous DNA molecule comprises a transcribable DNA sequence operably linked to a plant-expressible promoter. In certain embodiments, the transgenic plant is a N. tabacum plant; the transgenic plant and the scion are non-isogenic; the transgenic plant and the scion are of the same plant species; or the transgenic plant and the scion are of different plant species. In other embodiments, the site-specific endonuclease is selected from the group consisting of: an RNA-guided nuclease, a zinc-finger nuclease, a meganuclease, a TALE-nuclease, a recombinase, a transposase, and combinations of any thereof; is an RNA-guided nuclease comprising a Cas nuclease, a Cpf1 nuclease, or a variant of either thereof; or comprises a sequence having at least 85%, 90%, 91%, 92%, 93%, 94%, 95%, 96%, 97%, 98%, or 99% identity to a polypeptide selected from the group consisting of SEQ ID NOs: 37, 39, 40, and 41. In specific embodiments, the genomic modification is in a transcribable region of the genome; or in a non-transcribable region of the genome. In still other embodiments, the genomic modification confers an altered phenotype as compared to the phenotype of an otherwise isogenic plant that lacks the modification.

Also provided is a recombinant DNA construct encoding a site-specific endonuclease operably linked to a protein having at least 85%, 90%, 91%, 92%, 93%, 94%, 95%, 96%, 97%, 98%, or 99% identity to a polypeptide selected from the group consisting of SEQ ID NO:42 and SEQ ID NO:46. In some embodiments, the sequence is stably transformed into the genome of a plant. In yet another aspect, the invention provides a method for producing a transgenic plant cell, comprising transforming a plant cell with at least one DNA molecule or vector comprising the recombinant DNA construct to produce one or more transformed plant cells comprising the recombinant DNA construct stably transformed into the genome of the one or more transformed plant cells. In some embodiments, said plant cell is transformed via Agrobacterium-mediated transformation or Rhizobium-mediated transformation; said plant cell is transformed via microprojectile-mediated transformation or particle bombardment-mediated transformation; said plant cell is transformed via microinjection-mediated delivery of transgenic elements; said plant cell is transformed via polyethylene glycol-mediated delivery of transgenic elements; said plant cell is transformed via electroporation-mediated delivery of transgenic elements; said plant cell is transformed via nanoparticle-mediated delivery of transgenic elements. In specific embodiments, the nanoparticle can be a carbon nanotube, clay nanosheet, DNA nanostructure, or peptide nanoparticle. In other embodiments, said transgenic plant cell is a tobacco plant cell or a derivative thereof, e.g. a protoplast.

In another aspect, provided herein is a method of regenerating a plant, the method comprising: obtaining at least a first cell from a plant comprising a recombinant DNA construct encoding a plant selectable marker gene operably linked to a viral protein, wherein the recombinant DNA construct is not stably integrated into at least the first cell; and regenerating a plant comprising the recombinant DNA construct from at least the first cell in the presence of a selection agent. In some embodiments, the selectable marker gene is an antibiotic resistance gene, a herbicide resistance gene, or a metabolic resistance gene. In other embodiments, regeneration occurs by organogenesis or somatic embryogenesis.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 Schematic of plant transformation vectors. pFGR27 comprises TRV1 cloned in pCambia2300; pFGR28 comprises TRV2 with multiple cloning site (MCS) cloned in pCambia2300; pFGR29 comprises Cas9 cloned in pFGR28; pFGR32 comprises Cas9 and gRNA targeting the phytoene desaturase (PDS) gene cloned into pCambia 2300. Binary vectors pFGR27 and pFGR32 were used together to generate transgenic gene editing reagent donor lines or rootstock.

FIG. 2 shows editing of the phytoene desaturase (PDS) gene in Nicotiana tabacum. Panels A-E show Nicotiana tabacum lines transformed with the following plant transformation vectors: Panel A. pCambia 2300 control vector; Panel B. pFGR27+28 (no gene editing expected); Panel C. pFGR27+29 (no gene editing expected); Panel D. pFGR27+32 (gene editing expected); Panel E. pVIG12 (gene editing expected).

FIG. 3 shows high-throughput sequencing of the target region within the NtPDS gene in WT Scion (Panel A); and Nt-pFRG27+32-6 (Panel B) event used as a rootstock. The binding site for gRNA and PAM sites are marked by blue and green boxes, respectively. The image shows three replicates for each sample. Panel C shows the percentage of reads with edits in the Leaf 1 of WT Scion and Nt-pFRG27+32-6 rootstock.

FIG. 4 shows high-throughput sequencing of the target region within the NtPDS gene in WT Scion (Panel A) and Nt-pFRG27+32-6 (Panel B) event used as a rootstock. The binding site for gRNA and PAM sites are marked by blue and green boxes, respectively. The image shows three replicates for each sample. Panel C shows the percentage of reads with edits in the Leaf 2 of WT Scion and Nt-pFRG27+32-6 rootstock.

FIG. 5 shows high-throughput sequencing of the target region within the NtPDS gene in WT Scion (Panel A) and Nt-pFRG27+32-6 (Panel B) event used as rootstock. The binding site for gRNA and PAM sites are marked by blue and green boxes, respectively. The image shows three replicates for each sample. Panel C shows the percentage of reads with edits in the Leaf 3 of WT Scion and Nt-pFRG27+32-6 rootstock.

FIG. 6 shows high-throughput sequencing of the target region within the NtPDS gene in WT Scion (Panel A) and Nt-pFRG27+32-7 (Panel B) event used as a rootstock. The binding site for gRNA and PAM sites are marked by blue and green boxes, respectively. The image shows three replicates for each sample. Panel C shows percentage of reads with edits in the Leaf 1 of WT Scion and Nt-pFRG27+32-7 rootstock.

FIG. 7 shows high-throughput sequencing of the target region within the NtPDS gene in WT Scion (Panel A) and Nt-pFRG27+32-7 (Panel B) event used as rootstock. The binding site for gRNA and PAM sites are marked by blue and green boxes, respectively. The image shows three replicates for each sample. Panel C shows percentage of reads with edits in the Leaf 2 of WT Scion and Nt-pFRG27+32-7 rootstock.

FIG. 8 shows high-throughput sequencing of the target region within the NtPDS gene in WT Scion (Panel A) and Nt-pFRG27+32-7 (Panel B) event used as rootstock. The binding site for gRNA and PAM sites are marked by blue and green boxes, respectively. The image shows three replicates for each sample. Panel C shows percentage of reads with edits in the Leaf 3 of WT Scion and Nt-pFRG27+32-7 rootstock.

FIG. 9 shows high-throughput sequencing of the target region within the NtPDS gene in WT Scion (Panel A) and Nt-pFRG27+32-8 (Panel B) event used as rootstock. The binding site for gRNA and PAM sites are marked by blue and green boxes, respectively. The image shows three replicates for each sample. Panel C shows percentage of reads with edits in the Leaf 1 of WT Scion and Nt-pFRG27+32-8 rootstock.

FIG. 10 shows high-throughput sequencing of the target region within the NtPDS gene in WT Scion (Panel A) and Nt-pFRG27+32-8 (Panel B) event used as rootstock. The binding site for gRNA and PAM sites are marked by blue and green boxes, respectively. The image shows three replicates for each sample. Panel C shows percentage of reads with edits in the Leaf 2 of WT Scion and Nt-pFRG27+32-8 rootstock.

FIG. 11 shows high-throughput sequencing of the target region within the NtPDS gene in Wt Scion (Panel A) and Nt-pFRG27+32-8 (Panel B) event used as rootstock. The binding site for gRNA and PAM sites are marked by blue and green boxes, respectively. The image shows three replicates for each sample. Panel C shows percentage of reads with edits in the Leaf 3 of WT Scion and Nt-pFRG27+32-8 rootstock.

FIG. 12 shows PCR results for detection of NtPDSNptII gene. Panel A-Lanes 1-3 Nt-pCambia2300; Lanes 4-6 NtpFGR27+28; Lanes 7-9 Nt-pFGR27+29; Lanes 10-12 Nt-pFGR27+32; Lanes 13-15 pVIG12; Lanes 16-18 WT N. tabacum; Lanes 19-21-Negative control. Panel B shows PCR detection of NtPDS gene-Lanes 1-3 Nt-pCambia2300; Lanes 4-6 NtpFGR27+28; Lanes 7-9 Nt-pFGR27+29; Lanes 10-12 Nt-pFGR27+32; Lanes 13-15 pVIG12; Lanes 16-18 WT N. tabacum; Lanes 19-21-Negative control. Panel C shows a representative DNA sequence electropherogram obtained from one of the Nt-pFGR27+32 lines demonstrating albino phenotype.

FIG. 13 shows delivery of gene editing reagents across graft junction. Rootstock-Nt-pFGR27+32-Plant 6; Scion-WT Nicotiana tabacum, Rootstock-Nt-pFGR27+32-Plant 7; Scion-WT N. tabacum, Rootstock-Nt-pFGR27+32-Plant 8; Scion-WT N. tabacum. Self-grafted WT N. tabacum. L-Leaf.

FIG. 14 shows PCR verification for the presence of gene editing components: TRV RNA1, gRNA, Cas9 and TRV RNA 2. WT N. tabacum; WT Scion N. tabacum 6; WT Scion Nicotiana tabacum-7; WT Scion Nicotiana tabacum-8; Rootstock-Nt-pFGR27+32-6; Rootstock-Nt-pFGR27+32-7; Rootstock-Nt-pFGR27+32-8; + positive control; − negative control. 1,2,3: Leaf 1, Leaf 2 and Leaf 3 respectively. Ladder 1 Kb DNA plus (NEB).

FIG. 15 shows percentage of reads with edits in the NtPDS gene. Sampled from three leaves each of the WT scion and three independent events obtained using pFGR27+32 construct. Rootstock-Nt-pFGR27+32 event number 6 with WT scion; B. Rootstock-Nt-pFGR27+32 event number 7 with WT scion; C. Rootstock-Nt-pFGR27+32 event number 8 with WT scion.

FIG. 16 shows somatic transmission of CRISPR reagents and maintenance of the edits following organogenesis. Panel A shows regeneration of leaves from WT Scion grafted on Nt-pFRG27+pFGR32-6, Nt-pFGR27+32-7 and Nt-pFGR27+32-8. Panel B shows shoots regenerated from the leaves of WT scion grafted on pFGR27+32-7. Panel C shows green shoots started to turn pale and albino-week 4. Panel D shows albino plants after eight weeks.

FIG. 17 shows PCR screening for NptII gene following organogenesis.

FIG. 18 shows alignment of DNA sequences or Illumina reads generated through high-throughput sequencing from three technical replicates following organogenesis.

FIG. 19 shows percentage of edited reads for three independent plants regenerated from WT scion grafted on pFGR27+32-7.

FIG. 20 shows delivery of gene editing reagents across interspecies graft junctions. For all the experiments, Nt pFGR27+32-6 was used a scion, which was grafted onto different plant species: (Panel A) Apple (Malus x domestica M11IL659), (Panel B) Blueberry (Vaccinium sect. Cyanoccocus Elliot L695), (Panel C) Pear (Pyrus communis OHxF97C658), (Panel D) Cherry (Prunus avium Gi12-C656), (Panel E) Grape (Vitis vinifera Paulsen C709), (Panel F) Tomato (Solanum lycopersicum cv Heinz) and (Panel G) Strawberry (Fragaria x ananassa Totem C683).

FIG. 21 shows PCR verification to detect individual gene editing components Cas9, TRV1, TRV2 and gRNA in WT tissue of several plant species grafted on Nt-pFGR27+32-6. Panel A. Apple (Malus x domestica M11IL659), Panel B. Blueberry (Vaccinium sect. Cyanoccocus Elliot L695), Panel C. Pear (Pyrus communis OHxF97C658), Panel D. Cherry (Prunus avium Gi12-C656), Panel E. Tomato (Solanum lycopersicum cv Heinz) and PCR from cDNA in Panel F. Cherry (Prunus avium Gi12-C656) and Panel G. Blueberry (Vaccinium sect. Cyanoccocus Elliot L695). Expected amplicon sizes: Cas9 5′ region—1.2 kb, Cas9 3′ region—1.6 Kb; TRV RNA2 5′ region—1.6 Kb, TRV RNA2 3′ region—400 bp, gRNA-400 bp; TRV RNA1 5′ region—1.6 Kb, TRV RNA1 3′ region—700 bp. M-1 Kb Molecular Marker (NEB).

FIG. 22 shows high-throughput sequencing of the target region within the NtPDS gene in WT samples. The binding site for gRNA and PAM sites are marked by blue and green boxes, respectively. The image shows three replicates demonstrating no edits as expected.

FIG. 23 shows shoots regenerated from the leaves of WT scion grafted on rootstock pFGR27+32-7. Panels A and B show albino and chimeric regenerants obtained on leaf regeneration media without Kanamycin. Panels C and D show albino regenerants obtained on leaf regeneration media with Kanamycin 100 mg/L.

FIG. 24 shows high-throughput sequencing of the target region within the NtPDS gene in plant 1 regenerated from WT Scion Leaf 1 (Panel A).

FIG. 25 shows high-throughput sequencing of the target region within the NtPDS gene in plant 1 regenerated from WT Scion Leaf 2 (Panel B).

FIG. 26 shows high-throughput sequencing of the target region within the NtPDS gene in plant 1 regenerated from WT Scion Leaf 3 (Panel C).

FIG. 27 shows albino plant obtained after 8 weeks (Panel D); and percentage of reads with edits in the 3 leaves obtained from Plant 1 (Panel E).

FIG. 28 shows high-throughput sequencing of the target region within the NtPDS gene in plant 2 regenerated from WT Scion Leaf 1 (Panel A).

FIG. 29 shows high-throughput sequencing of the target region within the NtPDS gene in plant 2 regenerated from WT Scion Leaf 2 (Panel B).

FIG. 30 shows high-throughput sequencing of the target region within the NtPDS gene in plant 2 regenerated from WT Scion Leaf 3 (Panel C).

FIG. 31 shows albino plant obtained after 8 weeks (Panel D); and percentage of reads with edits in the 3 leaves obtained from Plant 2 (Panel E).

FIG. 32 shows high-throughput sequencing of the target region within the NtPDS gene in plant 3 regenerated from WT Scion Leaf 1 (Panel A).

FIG. 33 shows high-throughput sequencing of the target region within the NtPDS gene in plant 3 regenerated from WT Scion Leaf 2 (Panel B)

FIG. 34 shows high-throughput sequencing of the target region within the NtPDS gene in plant 3 regenerated from WT Scion Leaf 3 (Panel C)

FIG. 35 shows chimeric plant obtained after 8 weeks (Panel D); and percentage of reads with edits in the 3 leaves obtained from Plant 3 (Panel E).

### BRIEF DESCRIPTION OF THE SEQUENCES

SEQ ID NO:1 is a binary plasmid (pCambia 2300) comprising TRV RNA2, Cas9, NtPDS gRNA, NptII as selectable marker for plant transformation, and is also referred to as pFGR32.

SEQ ID NO:2 is a binary plasmid (pCambia 2300) comprising TRV RNA2 and Cas9, NptII as a selectable marker for plant transformation, and is also referred to as pFGR29.

SEQ ID NO:3 is a binary plasmid (pCambia 2300) comprising TRV RNA2, NptII as selectable marker, and is also referred to as pFGR27.

SEQ ID NO:4 is a binary plasmid (YL 156) comprising TRV RNA1, NptII as selectable marker for plant transformation, and is also referred to as pFGR28.

SEQ ID NO:5 is a binary plasmid (YL 156) comprising TRV RNA1 with the original bar gene removed, and is also referred to as pFGR15.

SEQ ID NO:6 is a binary plasmid (pKSE401) comprising Cas9 and gRNA to target NtPDS gene in Nicotiana tabacum, and is also referred to as pVIG12.

SEQ ID NO:7 (a.k.a. FGRP277) is a forward primer corresponding to the NtPDS gene from Nicotiana tabacum.

SEQ ID NO:8 (a.k.a. FGRP278) is a reverse primer corresponding to the NtPDS gene from Nicotiana tabacum.

SEQ ID NO:9 (a.k.a. FGRP257) is a forward primer corresponding to Cas9.

SEQ ID NO:10 (a.k.a. FGRP258) is a forward primer corresponding to Cas9.

SEQ ID NO:11 (a.k.a. FGRP253) is a reverse primer corresponding to Cas9.

SEQ ID NO:12 (a.k.a. FGRP254) is a reverse primer corresponding to Cas9.

SEQ ID NO:13 (a.k.a. FGRP259) is a forward primer corresponding to the gene coding for TRV RNA2 coat protein.

SEQ ID NO:14 (a.k.a. FGRP260) is a reverse primer corresponding to the gene coding for TRV RNA2 coat protein.

SEQ ID NO:15 (a.k.a. FGRP261) is a forward primer corresponding to the gene coding for 32.8K TRV RNA2 protein.

SEQ ID NO:16 (a.k.a. FGRP262) is a reverse primer corresponding to the gene coding for 32.8K TRV RNA2 protein.

SEQ ID NO:17 (a.k.a. RCWP374) is a forward primer corresponding to the gRNA promoter and scaffold.

SEQ ID NO:18 (a.k.a. RCWP375) is a reverse primer corresponding to the gRNA promoter and scaffold.

SEQ ID NO:19 (a.k.a. FGRP263) is a forward primer corresponding to the gene coding for replicase TRV RNA1 protein.

SEQ ID NO:20 (a.k.a. FGRP264) is a reverse primer corresponding to the gene coding for replicase TRV RNA 1 protein.

SEQ ID NO:21 (a.k.a. FGRP273) is a forward primer corresponding to the gene coding for 16 KDa TRV RNA1 protein.

SEQ ID NO:22 (a.k.a. FGRP274) is a reverse primer corresponding to the gene coding for 16 KDa TRV RNA1 protein.

SEQ ID NO:23 (a.k.a. FRGP5) is a forward primer corresponding to the NPTII gene.

SEQ ID NO:24 (a.k.a. FGRP56) is a reverse primer corresponding to the NPTII gene.

SEQ ID NO:25 (a.k.a. BWBP268) is a forward primer corresponding to NtPDS gRNA from N. tabacum.

SEQ ID NO:26 (a.k.a. BWBP269) is a reverse primer corresponding to NtPDS gRNA from N. tabacum.

SEQ ID NO:27 (a.k.a. FGRP3) is a forward primer to amplify the NptII gene from pCambia2300.

SEQ ID NO:28 (a.k.a. FGRP4) is a reverse primer to amplify the NptII gene from pCambia2300, with SbfI restriction site added.

SEQ ID NO:29 (a.k.a. FGRP165) is a forward primer to amplify TRV RNA2 genome.

SEQ ID NO:30 (a.k.a. FGRP166) is a reverse primer to amplify TRV RNA2 genome with PmeI restriction digestion recognition site added.

SEQ ID NO:31 (a.k.a. FGRP8) is a forward primer for testing the presence of Cas9 in Nicotiana tabacum plants.

SEQ ID NO:32 (a.k.a. FGRP9) is a reverse primer for testing the presence of whole Cas9 in Nicotiana tabacum plants.

SEQ ID NO:33 (a.k.a. RGP110) is a forward primer for testing the presence of TRV1 RNA1 genome in Nicotiana tabacum plants.

SEQ ID NO:34 (a.k.a. FGR159) is a reverse primer for testing the presence of TRV RNA1 genome in Nicotiana tabacum plants.

SEQ ID NO:35 (a.k.a. RGP111) is a forward primer for testing the presence of TRV RNA2 genome in Nicotiana tabacum plants.

SEQ ID NO:36 (a.k.a. RGP112) is a reverse primer for testing the presence of TRV RNA2 genome in Nicotiana tabacum plants.

SEQ ID NO:37 is a Streptococcus pyogenes Cas9 amino acid sequence (NCBI accession number MT670352).

SEQ ID NO:38 is a gRNA NtPDS sequence derived from NCBI accession number XM_016642616.

SEQ ID NO:39 is a Staphylococcus aureus Cas 9 (SaCas9) nuclease amino acid sequence (NCBI accession number AYD60528.1).

SEQ ID NO:40 is a Cas @ nuclease amino acid sequence (NCBI accession number 7LYS).

SEQ ID NO:41 is a CRISPR Prevotella and Francisella 1 nuclease amino acid sequence (NCBI accession number WBC51234.1).

SEQ ID NO:42 is an amino acid sequence for TRV2 viral coat protein (NCBI accession number UVC58663.1).

SEQ ID NO:43 is an amino acid sequence for TRV1 movement protein (NCBI accession number QYF10871.1).

SEQ ID NO:44 is an amino acid sequence for TRV1 replication protein (NCBI accession number NP_620669.1).

SEQ ID NO:45 is an amino acid sequence for TRV1 16 KDa protein (NCBI accession number NP_620672.1).

SEQ ID NO:46 is a partial amino acid sequence for TRV2 32.8 KDa protein (NCBI accession number WAK98717.1).

### DETAILED DESCRIPTION

Major genome editing technologies currently used for plant genome engineering include, for example, engineered zinc finger nucleases (ZFN); engineered transcription activator-like effector nucleases (TALEN); and the clustered regularly interspaced short palindromic repeat (CRISPR)/CRISPR-associated protein 9 nuclease (Cas9). Although the CRISPR/Cas9 system has emerged as the most widely used genome-editing technique, all genome editing technologies ultimately rely on the effective delivery of the genome editing components to the plant cell. Unfortunately, current methods remain constrained to easily transformed plant species using time and labor-intensive in vitro techniques. Additionally, in many instances, plant lines lacking transgenic DNA expressing the genome editing components are desired following gene editing. Therefore, the generation of stable gene-edited plant lines using (CRISPR)-CRISPR-associated protein 9 (Cas9) currently requires a lengthy process of outcrossing to eliminate CRISPR-Cas9-associated sequences used to produce the genomic modification. Moreover, this approach is incompatible with several high-value, obligate outcrossing crops; and while some tissue culture-free methods have been reported, their application remains restricted to model systems. Thus, there is a continuing need to develop novel compositions and methods to deliver gene editing components to plants that result in increased editing efficiencies while avoiding the need for subsequent manipulation to remove transgenic elements used to produce the genomic modification(s).

Viruses such as Tobacco Rattle Virus (TRV) infect meristematic tissue, spread over the vascular system in the plants at early stages of development, and have a broad host range. See, e.g., Wu and Cheng, Traffic. 2020 December; 21 (12): 725-736. These characteristics make viral delivery of gene editing components an attractive approach. However, delivery of a complete CRISPR/Cas9 complex using RNA viruses into the plant cell has not been reported, likely due to apparent technical limitations e.g. most RNA viruses are not believed to be able to carry extraneous information for coding for a protein as large as Cas9. As such, only viral delivery of individual gene-editing components has been previously demonstrated. For example, the RNA2 from TRV was previously used to introduce a gRNA sequence into a Cas9 transgenic plant to provide immunity against Tomato Yellow Leaf Curl Virus (TYLC) (Ali, Z. et al. Genome Biol. 16, 1-11 (2015)), but subsequent outcrossing is still required to eliminate one or more transgenic elements used to produce the genomic modification in these situations. The present disclosure represents a significant advance in the art in that it provides for complete CRISPR/Cas9 delivery from a transgenic plant or rootstock therefrom to a scion. In particular, provided herein are methods and compositions for delivering components required for gene-editing (a.k.a. gene-editing reagents) across a graft junction to plants lacking the genome editing components.

The methods and compositions of the present disclosure take advantage of the ability of plant viruses to spread systemically via the vasculature. Provided herein in one embodiment is therefore a method to deliver gene editing reagents across a graft junction, from a transgenic plant comprising a recombinant DNA construct encoding a site-specific endonuclease operably linked to a viral protein, or a rootstock therefrom to a grafted scion lacking said recombinant DNA construct and selecting at least a first cell from said scion comprising a genomic modification resulting from the presence of said site-specific endonuclease, bypassing post-editing transgene elimination in the scion. As such, plants comprising the recombinant DNA constructs of the present disclosure may be employed as donors to transport the gene-editing reagents across intra-species as well as inter-species graft junctions to facilitate efficient plant genome modification(s). As described herein, the plant genome modifications are also maintained in the somatic generation obtained via organogenesis. The present disclosure thus expands the horizons of plant gene editing, offering significant potential for enhancing crop diversity and agricultural productivity.

### I. Plant Grafting

Plant grafting, also known as “grafting” or “graft,” is a horticultural technique that involves merging the vascular tissue of one plant with another plant. This results in a single grafted plant, achieved through the inosculation of their vascular tissue. Grafted plants offer numerous advantages, such as increased plant vigor, improved disease resistance, enhanced tolerance to environmental stresses, and higher crop yields that extend over a longer harvest period. Furthermore, plant grafting can aid in the prevention of other diseases or disorders, including early blight (Altemaria solani), late blight (Phytophthora infestans), and blossom end-rot (a physiological disorder caused by low calcium levels). Grafted plants also demonstrate improved tolerance to environmental stressors like salinity or extreme temperatures. Methods for plant grafting are known in the art. See, e.g., Koepke T et al. (2013) Plant Cell Rep 32 (9): 1321-1337; Mudge K, et al. (2009) Horticultural reviews. John Wiley & Sons, Inc, NJ, pp 437-493; and Ruiz J M et al. (2005) Physiol Plant 124 (4): 465-475. Furthermore, tobacco in particular is graft compatible with several plant species. See, e.g. Molina-Hidalgo, F. J. et al. Trends Biotechnol. 2021 and Goldschmidt, E. E. et al. Front. Plant Sci. 2014.

The vascular system of a plant plays a crucial role in transporting water, minerals (through the xylem), and sugars (through the phloem) to the growing parts of the plant known as sinks. Additionally, the vascular system, particularly the phloem, facilitates the transport of macromolecules such as RNA and protein for long-distance signaling, controlling various plant functions. This process involves the synthesis of macromolecules in the companion cells of the phloem found in leaves and roots (known as sources), followed by the loading of these macromolecules into sieve elements. The macromolecules are then transported through plasmodesmata to reach distant growing parts of the plant (sinks). In accordance with the present disclosure, expressing genome editing reagents in a transgenic plant or rootstock therefrom can aid in the long-distance transport of transgenic RNA, DNA, and/or protein to a scion plant.

The grafting of plant materials comprising the recombinant DNA constructs disclosed herein onto plant materials lacking said recombinant DNA constructs, according to the present disclosure, enables the efficient delivery of genome editing reagents to plant cells for modifying a plant genome. This approach eliminates the requirement of preparing and delivering one or more genome editing reagents directly to plant cells via traditional in vitro techniques, broadening the range of editable plant genotypes. In some embodiments the scion comprising a genomic modification resulting from the presence of said site-specific endonuclease may be cleaned of viral sequences, including recombinant sequences delivered across the graft junction. For example, a scion plant or plant cell comprising a genomic modification may be cleaned of viral sequences using cryotherapy, heat therapy, chemotherapy, or any other method of virus cleaning known in the art. See, e.g. Wang, M R et al. (2018) Plant Methods 14, 87; and Gong, H. et al. (2019) Am. J. Potato Res. 96, 379-389.

A plant or scion that may be grafted with a transgenic plant comprising a recombinant DNA construct of the present disclosure, may include a variety of flowering plants or angiosperms, which may be further defined as including various dicotyledonous (dicot) plant species or monocotyledonous (monocot) plant species. A dicot plant could be tobacco, tomato, potato, apple, pear, cherry, cassava, sugar beet, spinach, lettuce, sunflower, rose, peach, almond, walnut, pecan, pistachio, legumes, radish, grape, strawberry, papaya, mango, avocado, hazelnut, pea, raspberry, blackberry, blueberry, or carrot. A monocot plant could be rice, wheat, sorghum, tulip, corn, switchgrass, bamboo, turf grasses, garlic, onion, coconut, palms, dates, barley, pineapple, banana, or sugarcane. Given that the present disclosure may apply to a broad range of plant species, the present disclosure further applies to other botanical structures analogous to pods of leguminous plants, such as bolls, siliques, fruits, nuts, tubers, etc. In specific embodiments, the plant may be a plant species capable of clonal propagation.

### II. Genome Editing

The present disclosure provides, in certain embodiments, methods of modifying a plant genome through site-specific integration or genome editing. Genome editing can be used to make one or more edit(s) or mutation(s) at a desired target site in the genome of a plant, such as to change expression and/or activity of one or more genes, or to integrate an insertion sequence or transgene at a desired location in a plant genome. Any site or locus within the genome of a plant may potentially be chosen for making a genomic edit (or gene edit) or site-directed integration of a transgene, construct, or transcribable DNA sequence. As used herein, a “target site” for genome editing or site-directed integration refers to the location of a polynucleotide sequence within a plant genome that is bound and cleaved by a site-specific nuclease to introduce a double-stranded break (DSB) or single-stranded nick into the nucleic acid backbone of the polynucleotide sequence and/or its complementary DNA strand within the plant genome. A target site may comprise, for example, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 29, or at least 30 consecutive nucleotides. A “target site” for an RNA-guided nuclease may comprise the sequence of either complementary strand of a double-stranded nucleic acid (DNA) molecule or chromosome at the target site. A site-specific nuclease may bind to a target site, such as via a non-coding guide RNA (e.g., without being limiting, a CRISPR RNA (crRNA) or a single-guide RNA (sgRNA) as described further herein). A non-coding guide RNA provided herein may be complementary to a target site (e.g., complementary to either strand of a double-stranded nucleic acid molecule or chromosome at the target site). It will be appreciated that perfect identity or complementarity may not be required for a non-coding guide RNA to bind or hybridize to a target site. For example, at least 1, at least 2, at least 3, at least 4, at least 5, at least 6, at least 7, or at least 8 mismatches (or more) between a target site and a non-coding RNA may be tolerated. A “target site” also refers to the location of a polynucleotide sequence within a plant genome that is bound and cleaved by any other site-specific nuclease that may not be guided by a non-coding RNA molecule, such as a zinc finger nuclease (ZFN), a transcription activator-like effector nuclease (TALEN), a meganuclease, etc., to introduce a DSB or single-stranded nick into the polynucleotide sequence and/or its complementary DNA strand. As used herein, a “target region” or a “targeted region” refers to a polynucleotide sequence or region that is flanked by two or more target sites. Without being limiting, in some embodiments a target region may be subjected to a mutation, deletion, insertion, substitution, inversion, transposition or duplication. As used herein, “flanked” when used to describe a target region of a polynucleotide sequence or molecule, refers to two or more target sites of the polynucleotide sequence or molecule surrounding the target region, with one target site on each side of the target region.

As used herein, a “targeted genome editing technique” refers to any method, protocol, or technique that allows the precise and/or targeted editing of a specific location in a genome of a plant (i.e., the editing is largely or completely non-random) using a site-specific nuclease, such as a meganuclease, a zinc-finger nuclease (ZFN), an RNA-guided endonuclease (e.g., the CRISPR/Cas9 system or the CRISPR/Cpf1 system), a TALE (transcription activator-like effector)-endonuclease (TALEN), a recombinase, or a transposase. As used herein, “editing” or “genome editing” refers to generating a targeted mutation, deletion, insertion, substitution, inversion, transposition or duplication of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 1000, at least 2500, at least 5000, at least 10,000, or at least 25,000 nucleotides of an endogenous plant genome nucleic acid sequence. As used herein, “editing” or “genome editing” may also encompass the targeted insertion or site-directed integration of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 750, at least 1000, at least 1500, at least 2000, at least 2500, at least 3000, at least 4000, at least 5000, at least 10,000, or at least 25,000 nucleotides into the endogenous genome of a plant. An “edit” or “genomic edit” in the singular refers to one such targeted mutation, deletion, insertion, substitution, inversion, or duplication, whereas “edits” or “genomic edits” refers to two or more targeted mutation(s), deletion(s), insertion(s), substitution(s), inversion(s), transposition(s) and/or duplication(s), with each “edit” being introduced via a targeted genome editing technique.

According to some embodiments, a site-specific nuclease may be co-delivered across a graft junction with a donor template molecule to serve as a template for making a desired edit, mutation or insertion into the genome at the desired target site through repair of the double strand break (DSB) or nick created by the site-specific nuclease. According to some embodiments, a site-specific nuclease may be co-delivered across a graft junction with a DNA molecule comprising a selectable or screenable marker gene either as a contiguous entity with the gene editing reagents, or as an independent transcribable sequence operably linked to a viral protein. In specific embodiments, the plant selectable marker gene is operably linked to a viral protein.

A site-specific nuclease provided herein may be selected from the group consisting of a zinc-finger nuclease (ZFN), a TALE-endonuclease (TALEN), a meganuclease, an RNA-guided endonuclease (e.g., Cas9 and Cpf1), a recombinase, a transposase, or any combination thereof. Sec, e.g., Khandagale et al. (Plant Biotechnol Rep 10:327-343, 2016); and Gaj et al. (Trends Biotechnol. 31 (7): 397-405, 2013). Zinc finger nucleases (ZFN) are synthetic proteins consisting of an engineered zinc finger DNA-binding domain fused to a cleavage domain (or a cleavage half-domain), which may be derived from a restriction endonuclease (e.g., FokI). The DNA binding domain may be canonical (C2H2) or non-canonical (e.g., C3H or C4). The DNA-binding domain can comprise one or more zinc fingers (e.g., 2, 3, 4, 5, 6, 7, 8, 9 or more zinc fingers) depending on the target site but may typically be composed of 3-4 (or more) zinc-fingers. Multiple zinc fingers in a DNA-binding domain may be separated by linker sequence(s). ZFNs can be designed to cleave almost any stretch of double-stranded DNA by modification of the zinc finger DNA-binding domain. ZFNs form dimers from monomers composed of a non-specific DNA cleavage domain (e.g., derived from the FokI nuclease) fused to a DNA-binding domain comprising a zinc finger array engineered to bind a target site DNA sequence. The amino acids at positions-1, +2, +3, and +6 relative to the start of the zinc finger α-helix, which contribute to site-specific binding to the target site, can be changed and customized to fit specific target sequences. The other amino acids may form a consensus backbone to generate ZFNs with different sequence specificities.

Methods and rules for designing ZFNs for targeting and binding to specific target sequences are known in the art. See, e.g., U.S. Patent App. Pub. Nos. 2005/0064474, 2009/0117617, and 2012/0142062. The FokI nuclease domain may require dimerization to cleave DNA and therefore two ZFNs with their C-terminal regions are needed to bind opposite DNA strands of the cleavage site (separated by 5-7 bp). The ZEN monomer can cut the target site if the two-ZF-binding sites are palindromic. A ZFN, as used herein, is broad and includes a monomeric ZFN that can cleave double stranded DNA without assistance from another ZFN. The term ZFN may also be used to refer to one or both members of a pair of ZFNs that are engineered to work together to cleave DNA at the same site. Because the DNA-binding specificities of zinc finger domains can be re-engineered using one of various methods, customized ZFNs can theoretically be constructed to target nearly any target sequence (e.g., at or near a gene in a plant genome). Publicly available methods for engineering zinc finger domains include Context-dependent Assembly (CoDA), Oligomerized Pool Engineering (OPEN), and Modular Assembly.

Transcription activator-like effectors (TALEs) can be engineered to bind practically any DNA sequence, such as at or near the genomic locus of a gene in a plant. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

TALENs are artificial restriction enzymes generated by fusing the TALE DNA binding domain to a nuclease domain. In some aspects, the nuclease is selected from a group consisting of PvuII, MutH, TevI, FokI, AlwI, MlyI, SbfI, SdaI, StsI, CleDORF, Clo051, and Pept071. When each member of a TALEN pair binds to the DNA sites flanking a target site, the FokI monomers dimerize and cause a double-stranded DNA break at the target site. The term TALEN, as used herein, is broad and includes a monomeric TALEN that can cleave double stranded DNA without assistance from another TALEN. The term TALEN also refers to one or both members of a pair of TALENs that work together to cleave DNA at the same site.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. PvuII, MutH, and TevI cleavage domains are useful alternatives to FokI and FokI variants for use with TALEs. PvuII functions as a highly specific cleavage domain when coupled to a TALE (see Yank et al., PLOS One 8: e82539, 2013). MutH is capable of introducing strand-specific nicks in DNA (see Gabsalilow et al., Nucleic Acids Research. 41: e83, 2013). TevI introduces double-stranded breaks in DNA at targeted sites (see Beurdeley et al., Nature Communications 4:1762, 2013).

The relationship between amino acid sequence and DNA recognition of the TALE binding domain allows for designable proteins. Software programs such as DNAWorks can be used to design TALE constructs. Other methods of designing TALE constructs are known to those of skill in the art. See Doyle et al. (Nucleic Acids Research 40: W117-122, 2012); Cermak et al. (Nucleic Acids Research 39: e82, 2011); and tale-nt.cac.cornell.edu/about. In another aspect, a TALEN provided herein is capable of generating a targeted DSB.

A site-specific nuclease may be a meganuclease. Meganucleases, which are commonly identified in microbes, such as the LAGLIDADG family of homing endonucleases, are unique enzymes with high activity and long recognition sequences (>14 bp) resulting in site-specific digestion of target DNA. Engineered versions of naturally occurring meganucleases typically have extended DNA recognition sequences (for example, 14 to 40 bp). The engineering of meganucleases can be more challenging than ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity.

A site-specific nuclease may be an RNA-guided nuclease. In an aspect, the targeted genome editing described herein may comprise the use of an RNA-guided endonuclease. As used herein, an “RNA-guided nuclease” refers to an RNA-guided DNA endonuclease associated with the CRISPR system. According to some embodiments, an RNA-guided endonuclease may be selected from the group consisting of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1 (also known as Cas12a, see e.g., Safari, F. et al., Cell Biosci 9:36, 2019), CasX, CasY, and homologs or modified versions of any thereof, as well as Argonaute proteins (non-limiting examples of Argonaute proteins include Thermus thermophilus Argonaute (TtAgo), Pyrococcus furiosus Argonaute (PfAgo), Natronobacterium gregoryi Argonaute (NgAgo), and homologs or modified versions of any thereof). According to some embodiments, an RNA-guided endonuclease is a Cas9 or Cpf1 enzyme. According to some embodiments, an RNA-guided endonuclease comprises a sequence having at least 85%, 90%, 91%, 92%, 93%, 94%, 95%, 96%, 97%, 98%, or 99% identity to a polypeptide selected from the group consisting of SEQ ID NOs: 37, 39, 40, and 41.

The CRISPR system, in its native context, provides bacteria and archaca with immunity to invading foreign nucleic acids and relies on an RNA-guided endonuclease to cleave the invading DNA or RNA into short sequence fragments and incorporating them into the bacterial CRISPR genomic locus. The incorporated short sequences, referred to as “protospacers”, and flanking direct repeats are transcribed and processed into CRISPR RNAs (crRNAs). These crRNAs hybridize with trans-activating crRNAs (tracrRNAs) to activate the RNA-guided Cas endonuclease to form a ribonucleoprotein (RNP) complex that is guided to a target site. A prerequisite for cleavage of the target site, however, is the presence of a conserved genomic protospacer-adjacent motif sequence recognized by the Cas endonuclease. A “protospacer adjacent motif” (PAM) herein refers to a short nucleotide sequence adjacent to a target sequence (protospacer) that is recognized (targeted) by a guide polynucleotide/Cas endonuclease system described herein. A PAM may be present in the genome immediately adjacent and upstream to the 5′ end of the genomic target site sequence complementary to the targeting sequence of the guide RNA—i.e., immediately downstream (3′) to the sense (+) strand of the genomic target site (relative to the targeting sequence of the guide RNA) as known in the art. See, e.g., Wu et al. (Quant Biol. 2 (2): 59-70, 2014). The Cas endonuclease may not successfully recognize a target DNA sequence if the target DNA sequence is not followed by a PAM sequence. The sequence and length of a PAM sequence herein can differ depending on the Cas endonuclease used. The PAM sequence can be of any length but is typically 1, 2, 3, 4, 5, 6, 7, 8, 9, 10, 11, 12, 13, 14, 15, 16, 17, 18, 19 or 20 nucleotides long.

CRISPR/Cas9, which is the CRISPR system from Streptococcus pyogenes, was adapted for use in eukaryotes and has been widely used for gene editing in plants. The CRISPR/Cas9 system requires both crRNA and tracrRNA to guide the Cas9 protein to recognize and cleave the target DNA double helix. Cas9 recognizes the genomic PAM sequence 5′-NGG-3′ (where N is any nucleotide) and, when located on the sense (+) strand adjacent to the target site, will create a blunt-end DSB at the target site, specifically the 5′-end of the PAM site. Cas9 has been observed to recognize other PAM sequences, such as 5′-NAG-3′ and 5′-NGA-3,′ which may result in cleavage of non-specific DNA sequences. However, the corresponding sequence of the guide RNA (i.e., immediately downstream (3′) to the targeting sequence of the guide RNA) may generally not be complementary to the genomic PAM sequence.

The CRISPR/Cpf1 system was discovered as an alternative to the CRISPR/Cas9 system for genome editing. While CRISPR/Cpf1 functions in a manner similar to CRISPR/Cas9, it requires only one crRNA molecule and no tracrRNA to cleave DNA. Cpf1 recognizes the genomic PAM sequence 5′-TTTV-3′ (where V is A, G, or C) or 5′-TTN-3′, depending on the Cpf1 ortholog. See e.g., Alok et al. (Front. Plant Sci. 11:264, 2020). When Cpf1 recognizes the genomic PAM located on the sense (+) strand adjacent to the target site, it will generate a staggered DSB with a 4 or 5-nt 5′ overhang at the target site, specifically the 3′-end of the PAM site.

The RNA-guided nuclease may be delivered as a protein with or without a guide RNA, or the guide RNA may be complexed with the RNA-guided nuclease enzyme and delivered as a ribonucleoprotein (RNP).

For RNA-guided endonucleases, a guide RNA molecule may be further provided to direct the endonuclease to a target site in the genome of the plant via base-pairing or hybridization to cause a DSB or nick at or near the target site. The guide RNA may be transformed or introduced into a plant cell or tissue as a gRNA molecule, or as a recombinant DNA molecule, construct or vector comprising a transcribable DNA sequence encoding the guide RNA operably linked to a promoter. As understood in the art, a guide RNA may comprise, for example, a CRISPR RNA (crRNA), a single-chain guide RNA (sgRNA), or any other RNA molecule that may guide or direct an endonuclease to a specific target site in the genome. A prototypical CRISPR associated protein, Cas9 from S. pyogenes, naturally binds two RNAs, a CRISPR RNA (crRNA) guide and a trans-acting CRISPR RNA (tracrRNA), to assemble a CRISPR ribonucleoprotein (crRNP). A “single-chain guide RNA” (or “sgRNA”) is an RNA molecule comprising a crRNA covalently linked to a tracrRNA by a linker sequence, which may be expressed as a single RNA transcript or molecule. The guide RNA comprises a guide or targeting sequence (also referred to herein as a “spacer sequence”) that is identical or complementary to a target site within the plant genome, such as at or near a gene. The guide RNA is typically a non-coding RNA molecule that does not encode a protein. The guide sequence of the guide RNA may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The guide sequence may be at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the genomic target site.

In addition to the guide sequence, a guide RNA may further comprise one or more other structural or scaffold sequence(s), which may bind or interact with an RNA-guided endonuclease. Such scaffold or structural sequences may further interact with other RNA molecules (e.g., tracrRNA). Methods and techniques for designing targeting constructs and guide RNAs for genome editing and site-directed integration at a target site within the genome of a plant using an RNA-guided endonuclease are known in the art.

For modification of a target gene through genome editing, an RNA-guided endonuclease may be targeted to a transcribable DNA sequence (i.e., a transcribable region) of said gene, such as a region of said gene comprising a coding sequence for a specific domain, an exon region, an intron region, or a combination thereof. For example, in certain embodiments a transcribable DNA sequence targeted for genome editing may comprise an exon/intron boundary or may be in close proximity to an exon/intron boundary. If the resulting modification spans an exon/intron boundary, the modification may be referred to as a modification in an exon region and an intron region. For genetic modification of the target gene, a guide RNA may be used, which comprises a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of the target gene or a sequence complementary thereto (e.g., 10, 11, 12, 13, 14, 15, 16, 17, 18, 19, 20, 21, 22, 23, 24, 25 or more consecutive nucleotides of the target gene or a sequence complementary thereto), although alternative splicing and different exon/intron boundaries may occur. As used herein, the term “consecutive” in reference to a polynucleotide or protein sequence means without deletions or gaps in the sequence.

According to some embodiments, the methods provided herein result in increased genomic editing efficiency in plant species known to exhibit low editing efficiencies, e.g. barley, B. oleracea, wheat, strawberry, grape, and corn. In particular embodiments, the methods provided may result in a gene editing efficiencies of at least 5%, at least 10%, at least 20%, at least 25%, at least 30%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 70%, at least 75%, at least 80%, at least 90%, or 100%. Such gene editing efficiencies percentages may be expressed, e.g. as the percentage of edited sequencing reads or any other standard measurement used in the art. This includes heterozygous or homozygous editing in haploid, diploid, triploid, tetraploid, hexaploid, octoploid, decaploid or polyploid plant species.

According to further embodiments, a plant is provided having a recombinant DNA molecule that yields an increase in editing efficiency in at least one plant cell by at least 5%, at least 10%, at least 20%, at least 25%, at least 30%, at least 40%, at least 50%, at least 60%, at least 70%, at least 75%, at least 80%, at least 90%, or 100%, as compared to a control plant.

As used herein, with respective to a given sequence, a “complement”, a “complementary sequence” and a “reverse complement” are used interchangeably. All three terms refer to the inversely complementary sequence of a nucleotide sequence, i.e., to a sequence complementary to a given sequence in reverse order of the nucleotides.

As used herein, the term “antisense” refers to DNA or RNA sequences that are complementary to a specific DNA or RNA sequence. Antisense RNA molecules are single-stranded nucleic acids which can combine with a sense RNA strand or sequence or mRNA to form duplexes due to complementarity of the sequences. The term “antisense strand” refers to a nucleic acid strand that is complementary to the “sense” strand. The “sense strand” of a gene or locus is the strand of DNA or RNA that has the same sequence as an RNA molecule transcribed from the gene or locus (with the exception of uracil in RNA and thymine in DNA).

A protospacer-adjacent motif (PAM) may be present in the genome immediately adjacent and upstream to the 5′ end of the genomic target site sequence complementary to the targeting sequence of the guide RNA—i.e., immediately downstream (3′) to the sense (+) strand of the genomic target site (relative to the targeting sequence of the guide RNA) as known in the art. See, e.g., Wu et al. (Quant Biol. 2 (2): 59-70, 2014). However, the corresponding sequence of the guide RNA (i.e., immediately downstream (3′) to the targeting sequence of the guide RNA) may generally not be complementary to the genomic PAM sequence.

In some embodiments, a site-specific nuclease is a recombinase. Non-limiting examples of recombinases that may be used include a serine recombinase attached to a DNA recognition motif, a tyrosine recombinase attached to a DNA recognition motif, or any recombinase enzyme known in the art attached to a DNA recognition motif. In certain embodiments, the site-specific nuclease is a recombinase or transposase, which may be a DNA transposase or recombinase attached or fused to a DNA binding domain. Non-limiting examples of recombinases include a tyrosine recombinase selected from the group consisting of a Cre recombinase, a Gin recombinase, a Flp recombinase, and a Tnp1 recombinase attached to a DNA recognition motif provided herein. In one aspect of the present disclosure, a Cre recombinase or a Gin recombinase provided herein is tethered to a zinc-finger DNA-binding domain, a TALE DNA-binding domain, or a Cas9 nuclease. In another aspect, a serine recombinase selected from the group consisting of a PhiC31 integrase, an R4 integrase, and a TP-901 integrase may be attached to a DNA recognition motif provided herein. In yet another aspect, a DNA transposase selected from the group consisting of a TALE-piggyBac and TALE-Mutator may be attached to a DNA binding domain provided herein.

Several site-specific nucleases, such as recombinases, zinc finger nucleases (ZFNs), meganucleases, and TALENs, are not RNA-guided and instead rely on their protein structure to determine their target site for causing the DSB or nick, or they are fused, tethered or attached to a DNA-binding protein domain or motif. The protein structure of the site-specific nuclease (or the fused/attached/tethered DNA binding domain) may target the site-specific nuclease to the target site. According to many of these embodiments, non-RNA-guided site-specific nucleases, such as recombinases, zinc finger nucleases (ZFNs), meganucleases, and TALENs, may be designed, engineered and constructed according to known methods to target and bind to a target site at or near the genomic locus of an endogenous gene of a plant to create a DSB or nick at such a genomic locus. The DSB or nick created by the non-RNA-guided site-specific nuclease may lead to knockdown of gene expression, or a change in the activity of the protein encoded by the endogenous gene, via repair of the DSB or nick, which may result in a mutation or insertion of a sequence at the site of the DSB or nick through cellular repair mechanisms. Such cellular repair mechanisms may be guided by a donor template molecule.

As used herein, a “donor molecule”, “donor template”, or “donor template molecule” (collectively a “donor template”), which may be a recombinant polynucleotide, DNA or RNA donor template or sequence, is defined as a nucleic acid molecule having a homologous nucleic acid template or sequence (e.g., homology sequence) and/or an insertion sequence for site-directed, targeted insertion or recombination into the genome of a plant cell via repair of a nick or DSB in the genome of a plant cell. A donor template may be a separate DNA molecule comprising one or more homologous sequence(s) and/or an insertion sequence for targeted integration, or a donor template may be a sequence portion (i.e., a donor template region) of a DNA molecule further comprising one or more other expression cassettes, genes/transgenes, and/or transcribable DNA sequences. For example, a “donor template” may be used for site-directed integration of a transgene or construct, or as a template to introduce a mutation, such as an insertion, deletion, substitution, etc., into a target site within the genome of a plant. A targeted genome editing technique provided herein may comprise the use of one or more, two or more, three or more, four or more, or five or more donor molecules or templates. A donor template provided herein may comprise at least one, at least two, at least three, at least four, at least five, at least six, at least seven, at least eight, at least nine, or at least ten gene(s) or transgene(s) and/or transcribable DNA sequence(s). Alternatively, a donor template may comprise no genes, transgenes or transcribable DNA sequences.

Without being limiting, a gene/transgene or transcribable DNA sequence of a donor template may include, for example, an insecticidal resistance gene, an herbicide tolerance gene, a nitrogen use efficiency gene, a water use efficiency gene, a yield enhancing gene, a nutritional quality gene, a DNA binding gene, a selectable marker gene, an RNAi or suppression construct, a site-specific genome modification enzyme gene, a single guide RNA of a CRISPR/Cas9 system, a geminivirus-based expression cassette, or a plant viral expression vector system. According to other embodiments, an insertion sequence of a donor template may comprise a protein encoding sequence or a transcribable DNA sequence that encodes a non-coding RNA molecule, which may target an endogenous gene for suppression. A donor template may comprise a promoter operably linked to a coding sequence, gene, or transcribable DNA sequence, such as a constitutive promoter, a tissue-specific or tissue-preferred promoter, a developmental stage promoter, or an inducible promoter. A donor template may comprise a leader, enhancer, promoter, transcriptional start site, 5′-UTR, a DNA sequence encoding a signal or targeting peptide, one or more exon(s), one or more intron(s), transcriptional termination site, region or sequence, 3′-UTR, and/or polyadenylation signal, which may each be operably linked to a coding sequence, gene (or transgene) or transcribable DNA sequence encoding a non-coding RNA, a guide RNA, an mRNA and/or protein. A donor template may be a single-stranded or double-stranded DNA or RNA molecule or plasmid.

An “insertion sequence” of a donor template is a sequence designed for targeted insertion into the genome of a plant cell, which may be of any suitable length. For example, the insertion sequence of a donor template may be between 2 and 50,000, between 2 and 10,000, between 2 and 5000, between 2 and 1000, between 2 and 500, between 2 and 250, between 2 and 100, between 2 and 50, between 2 and 30, between 15 and 50, between 15 and 100, between 15 and 500, between 15 and 1000, between 15 and 5000, between 18 and 30, between 18 and 26, between 20 and 26, between 20 and 50, between 20 and 100, between 20 and 250, between 20 and 500, between 20 and 1000, between 20 and 5000, between 20 and 10,000, between 50 and 250, between 50 and 500, between 50 and 1000, between 50 and 5000, between 50 and 10,000, between 100 and 250, between 100 and 500, between 100 and 1000, between 100 and 5000, between 100 and 10,000, between 250 and 500, between 250 and 1000, between 250 and 5000, or between 250 and 10,000 nucleotides or base pairs in length. A donor template may also have at least one homology sequence or homology arm, such as two homology arms, to direct the integration of a mutation or insertion sequence into a target site within the genome of a plant via homologous recombination, wherein the homology sequence or homology arm(s) are identical or complementary, or have a percent identity or percent complementarity, to a sequence at or near the target site within the genome of the plant. When a donor template comprises homology arm(s) and an insertion sequence, the homology arm(s) will flank or surround the insertion sequence of the donor template. Each homology arm may be at least 70%, at least 75%, at least 80%, at least 85%, at least 90%, at least 95%, at least 96%, at least 97%, at least 99% or 100% identical or complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a target DNA sequence within the genome of a plant.

Any method known in the art for site-directed integration may be used with the present disclosure. In the presence of a donor template molecule with an insertion sequence, the DSB or nick can be repaired by homologous recombination between homology arm(s) of the donor template and the plant genome, or by non-homologous end joining (NHEJ), resulting in site-directed integration of the insertion sequence into the plant genome to create the targeted insertion event at the site of the DSB or nick. Thus, site-specific insertion or integration of a transgene, transcribable DNA sequence, construct, or sequence may be achieved if the transgene, transcribable DNA sequence, construct or sequence is located in the insertion sequence of the donor template.

The introduction of a DSB or nick may also be used to introduce targeted mutations in the genome of a plant, including genomic modifications that reduce or disrupt the activity of the target gene, as compared to the activity of said gene in an otherwise identical plant, plant seed, plant part, or plant cell that lacks the modification. As used herein, a “mutation” refers to the permanent alteration of the nucleotide sequence of the genome of an organism, the extrachromosomal DNA, or other genetic elements. According to this approach, mutations, such as deletions, insertions, substitutions, inversions, and/or duplications may be introduced at a target site via imperfect repair of the DSB or nick to produce a genetic modification within a gene. Such mutations may be generated by imperfect repair of the targeted locus even without the use of a donor template molecule. A modification of a gene may be achieved by inducing a DSB or nick at or near the endogenous locus of the gene that results in expression of a non-functional protein, interfering protein, or a protein having reduced, disrupted, or altered activity as compared to a protein expressed from the gene lacking said modification.

As used herein, the term “insertion” as it relates to a mutation, refers to the addition of one or more extra nucleotides into the DNA. Insertions in the coding region of a gene may alter splicing of the mRNA (splice site mutation) or cause a shift in the reading frame (frameshift), both of which can significantly alter the gene product.

As used herein, the term “deletion” as it relates to a mutation refers to the removal of one or more nucleotides from the DNA. Like insertion mutations, these mutations can alter the reading frame of the gene.

As used herein, the term “substitution” as it relates to a mutation refers to an exchange of a single nucleotide for another.

As used herein, the term “inversion” refers to reversing the orientation of a chromosomal segment. An inversion can be accompanied by a loss of nucleotides flanking either one or both sites of the inversion due to DNA repair mechanisms occurring at the cut and ligation sites during the formation of an inversion.

As used herein, the term “duplication” refers to the creation of multiple copies of chromosomal regions, increasing the dosage of the genes located within them.

As used herein, the term “transposition” refers to the movement of a defined DNA segment from one genomic site to another.

As used herein, a “missense mutation” refers to a single nucleotide change that results in a codon that codes for a different amino acid. For example, the codon “CGU” encodes an arginine amino acid. If a missense mutation changes the G to a U, producing a “CUU” codon, the codon now encodes a leucine amino acid. Missense mutations can be caused by an insertion, deletion, substitution, duplication, or inversion. A “loss-of-function mutation” is a mutation in the coding sequence of a gene, which causes the function of the gene product, usually a protein, to be either reduced or completely absent. A loss-of-function mutation can, for instance, be caused by the truncation of the gene product. A phenotype associated with an allele with a loss of function mutation can be either recessive or dominant.

Similarly, such targeted mutations of a gene may be generated with a donor template molecule to direct a particular or desired mutation at or near the target site via repair of the DSB or nick. The donor template molecule may comprise a homologous sequence with or without an insertion sequence and comprising one or more mutations, such as one or more deletions, insertions, substitutions, inversions, and/or duplications, relative to the targeted genomic sequence at or near the site of the DSB or nick. For example, targeted mutations of a gene may be achieved by deleting, inserting, substituting, inverting, or duplicating at least a portion of the gene, such as by introducing a frame shift or premature stop codon into the coding sequence of the gene or introducing a modification into a transcribable DNA sequence. A deletion of a portion of a gene may also be introduced by generating DSBs or nicks at two target sites and causing a deletion of the intervening target region flanked by the target sites. A modification of a targeted gene may result in expression of a non-functional protein, interfering protein, or a protein having reduced, disrupted, or altered activity as compared to a protein expressed from the gene lacking said modification.

### II. Constructs for Genome Editing

Recombinant DNA constructs and vectors are provided comprising a polynucleotide sequence encoding a site-specific nuclease, such as a zinc-finger nuclease (ZFN), a meganuclease, an RNA-guided endonuclease, a TALE-endonuclease (TALEN), a recombinase, or a transposase, wherein the coding sequence is operably linked to a viral protein. For RNA-guided endonucleases, recombinant DNA constructs and vectors are further provided comprising a polynucleotide sequence encoding a guide RNA, wherein the guide RNA comprises a guide sequence of sufficient length having a percent identity or complementarity to a target site within the genome of a plant. A polynucleotide sequence of a recombinant DNA construct and vector that encodes a site-specific nuclease or a guide RNA may be operably linked to a plant expressible promoter, such as an inducible promoter, a constitutive promoter, a tissue-specific promoter, etc. In some embodiments, the site-specific endonuclease is operably linked to a viral protein, e.g. a viral protein from Tobacco Rattle Virus (TRV), Bean Yellow Dwarf Virus (BeYDV), Wheat Dwarf Virus (WDV), Tomato Mosaic Virus (ToMV), Pea Browning Virus (PEBV), Sonchus Yellow Net Rhabdovirus (SYNV), Potato Virus X (PVX), Foxtail Mosaic Virus (FoMV), Barley Yellow Striate Mosaic Virus (BYSMV), or Beet Necrotic Yellow Vein Virus (BNYVV). In other embodiments, the site-specific endonuclease is operably linked to a viral protein from, e.g. Cabbage Leaf Curl Virus (CaLCuV), Tomato Mottle Mosaic Virus (TMMV), Bean Golden Mosaic Virus (BGMV), Tomato golden mosaic virus (TGMV), or Cucumber Mosaic virus (CMV). In further embodiments, the recombinant DNA construct is stably transformed into the genome of a transgenic plant or a rootstock therefrom.

In an aspect, viral vectors comprising polynucleotides encoding a site-specific nuclease, and optionally one or more, two or more, three or more, or four or more gRNAs are provided to a plant cell by transformation methods known in the art (e.g., without being limiting, particle bombardment, PEG-mediated protoplast transfection or Agrobacterium-mediated transformation). In an aspect, vectors comprising polynucleotides encoding a Cpf1 nuclease, and optionally one or more, two or more, three or more, or four or more gRNAs are provided to a plant cell by transformation methods known in the art (e.g., without being limiting, particle bombardment, PEG-mediated protoplast transfection or Agrobacterium-mediated transformation). In another aspect, vectors comprising polynucleotides encoding a Cpf1 and, optionally one or more, two or more, three or more, or four or more crRNAs are provided to a cell by transformation methods known in the art (e.g., without being limiting, viral transfection, particle bombardment, PEG-mediated protoplast transfection or Agrobacterium-mediated transformation).

In another aspect, a second recombinant DNA construct encoding at least one viral replication protein; a movement protein; or a combination thereof may be used in accordance with the present disclosure. In some embodiments, the second recombinant DNA construct encodes a viral protein from, e.g. Tobacco Rattle Virus (TRV), Cabbage Leaf Curl Virus (CaLCuV), Bean Yellow Dwarf Virus (BcYDV), Pea early browning virus (PEBV), Tomato Mottle Mosaic Virus (TMMV), Bean Golden Mosaic Virus (BGMV), Tomato golden mosaic virus (TGMV), or Cucumber Mosaic virus (CMV). In other embodiments, the second recombinant DNA construct encodes a viral protein from, e.g. Wheat Dwarf Virus (WDV), Tomato Mosaic Virus (ToMV), Sonchus Yellow Net Rhabdovirus (SYNV), Potato Virus X (PVX), Foxtail Mosaic Virus (FoMV), Barley Yellow Striate Mosaic Virus (BYSMV), or Beet Necrotic Yellow Vein Virus (BNYVV). According to some embodiments, the second recombinant DNA comprises a sequence encoding a protein having at least 85%, 90%, 91%, 92%, 93%, 94%, 95%, 96%, 97%, 98%, or 99% identity to a polypeptide selected from the group consisting of SEQ ID NO:43, SEQ ID NO:44, and SEQ ID NO: 45. Without being limiting, a second recombinant DNA construct as provided herein, encoding at least one viral replication protein; a movement protein; or a combination thereof, may facilitate the transfer of recombinant sequences to neighboring plant cells directly through plasmodesmata. For example, a viral replication protein and/or movement protein encoded by a second recombinant DNA construct may facilitate the transfer of recombinant sequences encoding a site-specific endonuclease operably linked to a viral protein and/or at least one gRNA.

As used herein, a “gene” refers to a nucleic acid sequence forming a genetic and functional unit and coding for one or more sequence-related RNA and/or polypeptide molecules. A gene generally contains a coding region operably linked to appropriate regulatory sequences that regulate the expression of a gene product (e.g., a polypeptide or a functional RNA). A gene can have various sequence elements, including, but not limited to, a promoter, an untranslated region (UTR), exons, introns, and other upstream or downstream regulatory sequences.

As used herein, “locus” is a chromosomal locus or region where a polymorphic nucleic acid, trait determinant, gene, or marker is located. A “locus” can be shared by two homologous chromosomes to refer to their corresponding locus or region. As used herein, an “allele” refers to an alternative nucleic acid sequence of a gene or at a particular locus (e.g., a nucleic acid sequence of a gene or locus that is different than other alleles for the same gene or locus). Such an allele can be considered (i) wild-type or (ii) mutant if one or more mutations or edits are present in the nucleic acid sequence of the mutant allele relative to the wild-type allele. A mutant or edited allele for a gene may have reduced, disrupted, altered, or eliminated activity, or a reduced or eliminated expression level for the gene relative to the wild-type allele. For example, a mutant or edited allele for a target gene may have a deletion in the transcribable region of the endogenous target gene that reduces, disrupts, or alters the activity of the protein encoded by the mutant allele as compared to the activity of the protein encoded by the wild-type allele in an otherwise identical plant. For diploid organisms such as corn, a first allele can occur on one chromosome, and a second allele can occur at the same locus on a second homologous chromosome. If one allele at a locus on one chromosome of a plant is a mutant or edited allele and the other corresponding allele on the homologous chromosome of the plant is wild-type, then the plant is described as being heterozygous for the mutant or edited allele. However, if both alleles at a locus are mutant or edited alleles, then the plant is described as being homozygous or biallelic for the mutant or edited alleles. As used herein, the term “homozygous” refers to a genotype comprising two identical alleles at a given locus in a diploid genome. Given that corn is a diploid organism, CRISPR-mediated gene editing can result in biallelic (that is, different edits are made to the same locus on corresponding homologous chromosomes) edits resulting in a genotype comprising two non-identical mutant alleles at a given locus in a diploid genome in R₀ plants. When used in the context of edited alleles, plants comprising such genotypes may also be referred to as comprising a heteroallelic combination or biallelic edits.

As used herein, a “wild-type gene” or “wild-type allele” refers to a gene or allele having a sequence or genotype that is most common in a particular plant species, or another sequence or genotype having only natural variations, polymorphisms, or other silent mutations relative to the most common sequence or genotype that do not significantly impact the expression and activity of the gene or allele. Indeed, a “wild-type” gene or allele contains no variation, polymorphism, or any other type of mutation that substantially affects the normal function, activity, expression, or phenotypic consequence of the gene or allele relative to the most common sequence or genotype.

In general, the term “variant” refers to molecules with some differences, generated synthetically or naturally, in their nucleotide or amino acid sequences as compared to a reference (native) polynucleotides or polypeptides, respectively. These differences include substitutions, insertions, deletions, inversions, duplications, or any desired combinations of such changes in a native polynucleotide or amino acid sequence.

As used herein, the term “expression” refers to the biosynthesis of a gene product, and typically the transcription and/or translation of a nucleotide sequence, such as an endogenous gene, a heterologous gene, a transgene or an RNA and/or protein coding sequence, in a cell, tissue, organ, or organism, such as a plant, plant part or plant cell, tissue or organ.

The term “recombinant” in reference to a polynucleotide (DNA or RNA) molecule, protein, construct, vector, etc., refers to a polynucleotide or protein molecule or sequence that is man-made and not normally found in nature, and/or is present in a context in which it is not normally found in nature, including a polynucleotide (DNA or RNA) molecule, protein, construct, etc., comprising a combination of two or more polynucleotide or protein sequences that would not naturally occur together in the same manner without human intervention, such as a polynucleotide molecule, protein, construct, etc., comprising at least two polynucleotide or protein sequences that are operably linked but heterologous with respect to each other. For example, the term “recombinant” can refer to any combination of two or more DNA or protein sequences in the same molecule (e.g., a plasmid, construct, vector, chromosome, protein, etc.) where such a combination is man-made and not normally found in nature. As used in this definition, the phrase “not normally found in nature” means not found in nature without human introduction. A recombinant polynucleotide or protein molecule, construct, etc., can comprise polynucleotide or protein sequence(s) that is/are (i) separated from other polynucleotide or protein sequence(s) that exist in proximity to each other in nature, and/or (ii) adjacent to (or contiguous with) other polynucleotide or protein sequence(s) that are not naturally in proximity with each other.

Such a recombinant polynucleotide molecule, protein, construct, etc., can also refer to a polynucleotide or protein molecule or sequence that has been genetically engineered and/or constructed outside of a cell. For example, a recombinant DNA molecule can comprise any engineered or man-made plasmid, vector, etc., and can include a linear or circular DNA molecule. Such plasmids, vectors, etc., can contain various maintenance elements including a prokaryotic origin of replication and selectable marker gene, as well as one or more transgenes or expression cassettes perhaps in addition to a plant selectable marker gene, etc. The term “operably linked” refers to a functional linkage between a promoter or other regulatory element and an associated transcribable DNA sequence or coding sequence of a gene (or transgene), such that the promoter, etc., operates or functions to initiate, assist, affect, cause, and/or promote the transcription and expression of the associated transcribable DNA sequence or coding sequence, at least in certain cell(s), tissue(s), developmental stage(s), and/or condition(s).

Reference in this application to an “isolated DNA molecule” or an “isolated polynucleotide”, or an equivalent term or phrase, is intended to mean that the DNA molecule or polynucleotide is one that is present alone or in combination with other compositions, but not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” within the scope of this disclosure so long as the element is not within the genome of the organism and at the location within the genome in which it is naturally found. Similarly, a nucleotide sequence encoding a protein or any naturally occurring variant of that protein would be an isolated nucleotide sequence so long as the nucleotide sequence was not within the DNA of the organism in which the sequence encoding the protein is naturally found. A synthetic nucleotide sequence encoding the amino acid sequence of the naturally occurring protein would be considered to be isolated for the purposes of this disclosure. For the purposes of this disclosure, any transgenic nucleotide sequence, i.e., the nucleotide sequence of the DNA inserted into the genome of the cells of a plant or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

As commonly understood in the art, the term “promoter” can generally refer to a DNA sequence that contains an RNA polymerase binding site, transcription start site, and/or TATA box and assists or promotes the transcription and expression of an associated transcribable polynucleotide sequence and/or gene (or transgene). A promoter can be synthetically produced, varied or derived from a known or naturally occurring promoter sequence or other promoter sequence. A promoter can also include a chimeric promoter comprising a combination of two or more heterologous sequences. A promoter of the present disclosure can thus include variants or fragments of promoter sequences that are similar in composition, but not identical to, other promoter sequence(s) known or provided herein. A promoter provided herein, or variant or fragment thereof, may comprise a “minimal promoter” which provides a basal level of transcription and is comprised of a TATA box or equivalent DNA sequence for recognition and binding of the RNA polymerase II complex for initiation of transcription. A promoter can be classified according to a variety of criteria relating to the pattern of expression of an associated coding or transcribable sequence or gene (including a transgene) operably linked to the promoter, such as constitutive, developmental, tissue-specific, inducible, etc. Promoters that drive expression in all or most tissues of the plant are referred to as “constitutive” promoters. Promoters that drive expression during certain periods or stages of development are referred to as “developmental” promoters. Promoters that drive enhanced expression in certain tissues of the plant relative to other plant tissues are referred to as “tissue-enhanced” or “tissue-preferred” promoters. Thus, a “tissue-preferred” promoter causes relatively higher or preferential expression in a specific tissue(s) of the plant, but with lower levels of expression in other tissue(s) of the plant. Promoters that express within a specific tissue(s) of the plant, with little or no expression in other plant tissues, are referred to as “tissue-specific” promoters. An “inducible” promoter is a promoter that initiates transcription in response to an environmental stimulus such as cold, drought or light, or other stimuli, such as wounding or chemical application. A promoter can also be classified in terms of its origin, such as being heterologous, homologous, chimeric, synthetic, etc.

As used herein, a “plant-expressible promoter” refers to a promoter that can initiate, assist, affect, cause, and/or promote the transcription and expression of its associated transcribable DNA sequence, coding sequence or gene in a plant cell or tissue.

The term “heterologous” in reference to a promoter or other regulatory sequence in relation to an associated polynucleotide sequence (e.g., a transcribable DNA sequence or coding sequence or gene) is a promoter or regulatory sequence that is not operably linked to such associated polynucleotide sequence in nature without human introduction—e.g., the promoter or regulatory sequence has a different origin relative to the associated polynucleotide sequence and/or the promoter or regulatory sequence is not naturally occurring in a plant species to be transformed with the promoter or regulatory sequence.

As used herein, an “endogenous gene” or an “endogenous locus” refers to a gene or locus at its natural and original chromosomal location.

As used herein, in the context of a protein-coding gene, an “exon” refers to a segment of a DNA or RNA molecule containing information coding for a protein or polypeptide sequence.

As used herein, an “intron” of a gene refers to a segment of a DNA or RNA molecule, which does not contain information coding for a protein or polypeptide, and which is first transcribed into an RNA sequence but then spliced out from a mature RNA molecule.

As used herein, an “untranslated region (UTR)” of a gene refers to a segment of an RNA molecule or sequence (e.g., a mRNA molecule) expressed from a gene (or transgene), but excluding the exon and intron sequences of the RNA molecule. An “untranslated region (UTR)” also refers to a DNA segment or sequence encoding such a UTR segment of an RNA molecule. An untranslated region can be a 5′-UTR or a 3′-UTR depending on whether it is located at the 5′ or 3′ end of a DNA or RNA molecule or sequence relative to a coding region of the DNA or RNA molecule or sequence (i.e., upstream (5′) or downstream (3′) of the exon and intron sequences, respectively).

As used herein, a “signal peptide” or a “targeting peptide” refers to a short amino acid sequence, which directs a transcribed protein to a specific location within the cell, or a different location or tissue within the plant. In some embodiments, a signal peptide can direct a protein of interest linked thereto to a chloroplast, a mitochondria, a nucleus, an endoplasmic reticulum, a vacuole, a golgi bodies, a symplast, a apoplast, bark, suberized layers, a leaf, a stem, a root, a flower, an ovary, pollen, a fruit, a tuber, a silique, etc.

As used herein, a “transcribable region” or “transcribable DNA sequence” refers to a nucleic acid sequence expressed from a gene (or transgene).

As used herein, a “transcription termination sequence” refers to a nucleic acid sequence containing a signal that triggers the release of a newly synthesized transcript RNA molecule from an RNA polymerase complex and marks the end of transcription of a gene or locus.

Recombinant DNA molecules or constructs provided herein may be synthesized and modified by methods known in the art, either completely or in part, where it is desirable to provide sequences useful for DNA manipulation (such as restriction enzyme recognition sites or recombination-based cloning sites), plant-preferred sequences (such as plant-codon usage or Kozak consensus sequences), or sequences useful for DNA construct design (such as spacer or linker sequences). The present disclosure includes recombinant DNA molecules (or constructs) and proteins having at least 50% sequence identity, at least 60% sequence identity, at least 70% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, and at least 99% sequence identity to any of the recombinant DNA molecule or amino acid sequences provided herein. The terms “percent identity,” “% identity” or “percent identical” as used herein in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.” Sequences having a percent identity to a base sequence may exhibit the activity of the base sequence.

Degeneracy of the genetic code provides the possibility to substitute at least one base of the protein encoding sequence of a gene with a different base without causing the amino acid sequence of the polypeptide produced from the gene to be changed. When optimally aligned, homolog proteins, or their corresponding nucleotide sequences, have typically at least about 70%, at least about 75%, at least about 80%, at least about 85%, at least about 90%, at least about 92%, at least about 93%, at least about 94%, at least about 95%, at least about 96%, at least about 97%, at least about 98%, at least about 99%, or even at least about 99.5% identity over the full length of a protein or its corresponding nucleotide sequence identified as being associated with imparting an altered phenotype when expressed in plant cells.

Homologs are inferred from sequence similarity, by comparison of protein sequences, for example, manually or by use of a computer-based tool. For optimal alignment of sequences to calculate their percent identity, various pair-wise or multiple sequence alignment algorithms and programs are known in the art, such as ClustalW or Basic Local Alignment Search Tool® (BLAST), etc., that can be used to compare the sequence identity or similarity between two or more nucleotide or protein sequences. BLAST, can also be used, for example to search query protein sequences of a base organism against a database of protein sequences of various organisms, to find similar sequences. The generated summary Expectation value (E-value) can be used to measure the level of sequence similarity. Because a protein hit with the lowest E-value for a particular organism may not necessarily be an ortholog or be the only ortholog, a reciprocal query is used to filter hit sequences with significant E-values for ortholog identification. The reciprocal query entails search of the significant hits against a database of protein sequences of the base organism. A hit can be identified as an ortholog, when the reciprocal query's best hit is the query protein itself or a paralog of the query protein. With the reciprocal query process orthologs are further differentiated from paralogs among all the homologs, which allows for the inference of functional equivalence of genes.

The terms “percent complementarity” or “percent complementary”, as used herein in reference to two nucleotide sequences, is similar to the concept of percent identity but refers to the percentage of nucleotides of a query sequence that optimally base-pair or hybridize to nucleotides of a subject sequence when the query and subject sequences are linearly arranged and optimally base paired without secondary folding structures, such as loops, stems or hairpins. Such a percent complementarity may be between two DNA strands, two RNA strands, or a DNA strand and an RNA strand. The “percent complementarity” is calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences may be determined based on the known pairings of nucleotide bases, such as G-C, A-T, and A-U, through hydrogen bonding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present disclosure, when two sequences (query and subject) are optimally base-paired (with allowance for mismatches or non-base-paired nucleotides but without folding or secondary structures), the “percent complementarity” for the query sequence is equal to the number of base-paired positions between the two sequences divided by the total number of positions in the query sequence over its length (or by the number of positions in the query sequence over a comparison window), which is then multiplied by 100%.

As used herein, a “fragment” of a polynucleotide refers to a sequence comprising at least about 50, at least about 75, at least about 95, at least about 100, at least about 125, at least about 150, at least about 175, at least about 200, at least about 225, at least about 250, at least about 275, at least about 300, at least about 500, at least about 600, at least about 700, at least about 750, at least about 800, at least about 900, or at least about 1000 contiguous nucleotides, or longer, of a DNA molecule or protein as disclosed herein. Methods for producing such fragments from a starting promoter molecule are well known in the art. Fragments of a DNA molecule or protein may exhibit the activity of the DNA molecule or protein from which they are derived.

A plant selectable marker gene (or transgene) in a transformation vector or recombinant DNA construct of the present disclosure may be used to assist in the selection of cells or tissue due to the presence of a selection agent, such as an antibiotic or herbicide, wherein the plant selectable marker gene provides tolerance or resistance to the selection agent. Thus, the selection agent may bias or favor the survival, development, growth, proliferation, etc., of cells expressing the plant selectable marker gene, such as to increase the proportion of modified cells or tissues in the R₀ plant. As used herein, a “plant selectable marker” can refer to a protein encoded by a plant selectable marker gene, e.g. a neomycin phosphotransferase II enzyme encoded by nptII. According to some embodiments, the plant selectable marker gene can be expressed from a recombinant DNA construct that is not stably integrated into the genome of the plant. In specific embodiments, provided herein are recombinant DNA constructs comprising a sequence encoding a plant selectable marker operably linked to a viral protein. Commonly used plant selectable marker genes include, for example, those conferring tolerance or resistance to antibiotics, such as kanamycin and paromomycin (nptII), hygromycin B (aph IV), streptomycin or spectinomycin (aadA) and gentamycin (aac3 and aacC4), or those conferring tolerance or resistance to herbicides such as glufosinate (bar or pat), dicamba (DMO) and glyphosate (proA or EPSPS). Plant screenable marker genes may also be used, which provide an ability to visually screen for transformants, such as luciferase or green fluorescent protein (GFP), or a gene expressing a beta glucuronidase or uidA gene (GUS) for which various chromogenic substrates are known. Plant transformation may also be carried out in the absence of selection during one or more steps or stages of culturing, developing or regenerating transformed or modified explants, tissues, plants and/or plant parts.

### III. Transformation Methods

Methods and compositions are provided for transforming a plant cell, tissue or explant with a recombinant DNA molecule or construct encoding one or more molecules required for targeted genome editing (e.g., guide RNA(s) and/or site-directed nuclease(s)). Suitable methods for transformation of host plant cells include virtually any method by which DNA or RNA can be introduced into a cell (for example, where a recombinant DNA construct is stably integrated into a plant chromosome or where a recombinant DNA construct or an RNA is transiently provided to a plant cell) and are well known in the art. Two effective methods for cell transformation are bacterially-mediated transformation, such as Agrobacterium-mediated or Rhizobium-mediated transformation, and microprojectile or particle bombardment-mediated transformation. Microprojectile bombardment methods are illustrated, for example, in U.S. Pat. Nos. 5,550,318; 5,538,880; 6,160,208; and 6,399,861. Agrobacterium-mediated transformation methods are described, for example in U.S. Pat. No. 5,591,616. Other methods for plant transformation, such as microinjection, electroporation, vacuum infiltration, pressure, sonication, silicon carbide fiber agitation, PEG-mediated transformation, nanoparticle-mediated gene transformation (See, e.g. Lv et al. The Plant Journal (2020) 104, 880-891), etc., are also known in the art.

Transformation of plant material is practiced in tissue culture on nutrient media, for example a mixture of nutrients that allow cells to grow in vitro. Recipient cell targets include, but are not limited to, meristem cells, shoot tips, hypocotyls, calli, immature or mature embryos, and gametic cells such as microspores and pollen. Callus can be initiated from tissue sources including, but not limited to, immature or mature embryos, hypocotyls, seedling apical meristems, microspores and the like. Cells containing a transgenic nucleus are grown into transgenic plants, also referred to as R₀ plants. As used herein, “R₀ plant” refers to an initial regenerated transformant. As used herein, “R₁ seed” refers to the seed produced from selfing R₀ plants. As used herein, “R₁ plant” refers to a plant grown from R₁ seed. As used herein, “R₂ seed” refers to the seed produced from selfing R₁ plants. As used herein, “R₂ plant” refers to a plant grown from R₂ seed.

Any suitable method or technique for transformation of a plant cell known in the art may be used according to present methods. In transformation, DNA is typically introduced into only a small percentage of target plant cells in any one transformation experiment. Marker genes are used to provide an efficient system for identification of those cells that are stably transformed by receiving and integrating a recombinant DNA molecule into their genomes.

As used herein, the terms “regeneration” and “regenerating,” and like terms “generation” and “generating,” refer to a process of growing or developing a plant from one or more plant cells through one or more culturing steps. Transformed or edited cells, tissues or explants containing a DNA sequence insertion or edit may be grown, developed or regenerated into transgenic plants in culture, plugs, or soil according to methods known in the art. Certain embodiments of the disclosure, therefore, relate to methods and constructs for regenerating a plant from a cell with modified genomic DNA resulting from genome editing. The regenerated plant can then be used to propagate additional plants.

According to an aspect of the present disclosure, regenerated plants or a progeny plant, plant part or seed thereof can be screened or selected based on a marker, trait, or phenotype produced by the edit or mutation, or by the site-directed integration of an insertion sequence, transgene, etc., in the developed or regenerated plant, or a progeny plant, plant part or seed thereof. If a given mutation, edit, trait or phenotype is recessive, one or more generations or crosses (e.g., selfing) from the initial R₀ plant may be necessary to produce a plant homozygous for the edit or mutation so the trait or phenotype can be observed. Progeny plants, such as plants grown from R₁ seed or in subsequent generations, can be tested for zygosity using any known zygosity assay, such as by using a single nucleotide polymorphism (SNP) assay, DNA sequencing, thermal amplification, or polymerase chain reaction (PCR), and/or Southern blotting that allows for the distinction between heterozygote, homozygote and wild-type plants.

Methods and techniques are provided for screening for, and/or identifying, cells or plants, etc., for the presence of targeted edits or transgenes, and selecting cells or plants comprising targeted edits or transgenes, which may be based on one or more phenotypes or traits, or on the presence or absence of a molecular marker or polynucleotide or protein sequence in the cells or plants. As used herein, a “molecular technique” refers to any method known in the fields of molecular biology, biochemistry, genetics, plant biology, or biophysics that involves the usc, manipulation, or analysis of a nucleic acid, a protein, or a lipid. Without being limiting, molecular techniques useful for detecting the presence of a modified sequence in a genome include phenotypic screening; molecular marker technologies such as SNP analysis by TaqMan® or Illumina/Infinium technology; Southern blot; PCR (including amplicon sequencing which consists of the generation of one or more unique PCR products across the genomic region of interest for further sequencing analysis, e.g., using Next-Gen Sequencing techniques known in the art. Sequence data from each sample is then mapped to a reference sequence to identify consensus differences); enzyme-linked immunosorbent assay (ELISA); and sequencing (e.g., Sanger, Illumina®, 454, Pac-Bio, Ion Torrent™, Nanopore). In one aspect, a method of detection provided herein comprises phenotypic screening. In another aspect, a method of detection provided herein comprises SNP analysis. In a further aspect, a method of detection provided herein comprises a Southern blot. In a further aspect, a method of detection provided herein comprises PCR. In a further aspect, a method of detection provided herein comprises amplicon sequencing. In an aspect, a method of detection provided herein comprises ELISA. In a further aspect, a method of detection provided herein comprises determining the sequence of a nucleic acid or a protein. Without being limiting, nucleic acids can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY).

Nucleic acids can be isolated using techniques routine in the art. For example, nucleic acids can be isolated using any method including, without limitation, recombinant nucleic acid technology, and/or PCR. General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Detection (e.g., of an amplification product, of a hybridization complex, of a polypeptide) can be accomplished using detectable labels that may be attached or associated with a hybridization probe or antibody. The term “label” is intended to encompass the use of direct labels as well as indirect labels. Detectable labels include enzymes, prosthetic groups, fluorescent materials, luminescent materials, bioluminescent materials, and radioactive materials. The screening and selection of modified (e.g., edited) plants or plant cells can be through any methodologies known to those skilled in the art of molecular biology. Examples of screening and selection methodologies include, but are not limited to, Southern analysis, PCR amplification for detection of a polynucleotide (including amplicon sequencing), Northern blots, RNase protection, primer-extension, RT-PCR amplification for detecting RNA transcripts, Sanger sequencing, Next Generation sequencing technologies (e.g., Illumina®, PacBio®, Ion Torrent™, etc.) enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known in the art.

As used herein, the term “polypeptide” refers to a chain of at least two covalently linked amino acids. Polypeptides can be encoded by polynucleotides provided herein. An example of a polypeptide is a protein. Proteins provided herein can be encoded by nucleic acid molecules provided herein. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

Polypeptides can be detected using antibodies. Techniques for detecting polypeptides using antibodies include enzyme linked immunosorbent assays (ELISAs), Western blots, immunoprecipitations and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

### IV. Genome Modified Plants

As used herein, “modified” in the context of a plant, plant seed, plant part, plant cell, and/or plant genome, refers to a plant, plant seed, plant part, plant cell, and/or plant genome comprising an engineered change in the expression level and/or endogenous sequence of one or more genes of interest relative to a wild-type or control plant, plant seed, plant part, plant cell, and/or plant genome. Indeed, the term “modified” may further refer to a plant, plant seed, plant part, plant cell, and/or plant genome having one or more deletions and/or one or more nucleotide substitutions or nucleotide insertions affecting an endogenous gene introduced through chemical mutagenesis, transposon insertion or excision, or any other known mutagenesis technique, or introduced through genome editing. In an aspect, a modified plant, plant seed, plant part, plant cell, and/or plant genome can comprise one or more transgenes. For clarity, therefore, a modified plant, plant seed, plant part, plant cell, and/or plant genome includes a mutated, edited and/or transgenic plant, plant seed, plant part, plant cell, and/or plant genome having a modified sequence of a gene relative to a wild-type or control plant, plant seed, plant part, plant cell, and/or plant genome. Furthermore, the modification may reduce, disrupt, or alter the activity of the protein encoded by the gene as compared to the activity of the protein encoded by the gene in an otherwise identical plant.

Modified plants, plant parts, seeds, etc., may have been subjected to mutagenesis, genome editing or site-directed integration, genetic transformation, or a combination thereof. Such “modified” plants, plant seeds, plant parts, and plant cells include plants, plant seeds, plant parts, and plant cells that are offspring or derived from “modified” plants, plant seeds, plant parts, and plant cells that retain the molecular change (e.g., change in expression level and/or activity) to the gene. A modified seed provided herein may give rise to a modified plant provided herein. A modified plant, plant seed, plant part, plant cell, or plant genome provided herein may comprise a recombinant DNA construct or vector or genome edit as provided herein.

A “modified plant product” may be any product made from a modified plant, plant part, plant cell, or plant chromosome provided herein, or any portion or component thereof. For example, in some embodiments a modified plant product may be a commodity product produced from a modified plant or part thereof containing the recombinant DNA molecule as described herein. In some embodiments, commodity products contain a detectable amount of DNA comprising a DNA sequence selected from the group consisting of SEQ ID NOs: 1-6 or fragments or variants thereof; a DNA sequence encoding a site-specific endonuclease operably linked to a viral protein as provided herein, e.g. a site-specific endonuclease operably linked to a protein having at least 85%, 90%, 91%, 92%, 93%, 94%, 95%, 96%, 97%, 98%, or 99% identity to a polypeptide selected from the group consisting of SEQ ID NO:42 and SEQ ID NO:46. As used herein, a “commodity product” refers to any composition or product which is comprised of material derived from a modified plant, seed, plant cell, or plant part containing a DNA molecule as described herein, such as those provided as SEQ ID NOs: 1-6 or fragments or variants thereof; a DNA sequence encoding a site-specific endonuclease operably linked to a viral protein as provided herein, e.g. a site-specific endonuclease operably linked to a protein having at least 85%, 90%, 91%, 92%, 93%, 94%, 95%, 96%, 97%, 98%, or 99% identity to a polypeptide selected from the group consisting of SEQ ID NO:42 and SEQ ID NO:46. Commodity products include but are not limited to processed seeds, grains, plant parts, and meal, protein concentrate, protein isolate, grain, starch, flour, biomass, or seed oil. A commodity product containing a detectable amount of DNA corresponding to the recombinant DNA molecule as described herein, such as those provided as SEQ ID NOs: 1-6 or fragments or variants thereof; a DNA sequence encoding a site-specific endonuclease operably linked to a viral protein as provided herein, e.g. a site-specific endonuclease operably linked to a protein having at least 85%, 90%, 91%, 92%, 93%, 94%, 95%, 96%, 97%, 98%, or 99% identity to a polypeptide selected from the group consisting of SEQ ID NO:42 and SEQ ID NO:46 is contemplated. Detection of one or more of this DNA in a sample may be used for determining the content or the source of the commodity product. Any standard method of detection for DNA molecules may be used, including methods of detection disclosed herein.

Modified plants may be further crossed to themselves or other plants to produce modified plant seeds and progeny. A modified plant may also be prepared by crossing a first plant comprising a DNA sequence or construct or an edit (e.g., a genomic deletion) with a second plant lacking the DNA sequence or construct or edit. For example, a DNA sequence or inversion may be introduced into a first plant line that is amenable to transformation or editing, which may then be crossed with a second plant line to introgress the DNA sequence or edit (e.g., deletion) into the second plant line. Progeny of these crosses can be further backcrossed into the desirable line multiple times, such as through 6 to 8 generations or back crosses, to produce a progeny plant with substantially the same genotype as the original parental line, but for the introduction of the DNA sequence or edit. A modified plant, plant cell, or seed provided herein may be a hybrid plant, plant cell, or seed. As used herein, a “hybrid” is created by crossing two plants from different varieties, lines, inbreds, or species, such that the progeny comprises genetic material from each parent. Skilled artisans recognize that higher order hybrids can be generated as well.

A modified plant, plant part, plant cell, or seed provided herein may be of an elite variety or an elite line. An “elite variety” or an “elite line” refers to a variety that has resulted from breeding and selection for superior agronomic performance.

As used herein, the term “control plant” (or likewise a “control” plant seed, plant part, plant cell, and/or plant genome) refers to a plant (or plant seed, plant part, plant cell, and/or plant genome) that is used for comparison to a modified plant (or modified plant seed, plant part, plant cell, and/or plant genome) and has the same or similar genetic background (e.g., same parental lines, hybrid cross, inbred line, testers, etc.) as the modified plant (or plant seed, plant part, plant cell, and/or plant genome), except for genome edit(s) (e.g., a deletion) affecting a gene. For example, a control plant may be an inbred line that is the same as the inbred line used to make the modified plant, or a control plant may be the product of the same hybrid cross of inbred parental lines as the modified plant, except for the absence in the control plant of any transgenic events or genome edit(s) affecting a gene. Similarly, an “unmodified control plant” refers to a plant that shares a substantially similar or essentially identical genetic background as a modified plant, but without the one or more engineered changes to the genome (e.g., mutation or edit) of the modified plant. For purposes of comparison to a modified plant, plant seed, plant part, plant cell, and/or plant genome, a “wild-type plant” (or likewise a “wild-type” plant seed, plant part, plant cell, and/or plant genome) refers to a non-transgenic and non-genome edited control plant, plant seed, plant part, plant cell, and/or plant genome. As used herein, a “control” plant, plant seed, plant part, plant cell, and/or plant genome may also be a plant, plant seed, plant part, plant cell, and/or plant genome having a similar (but not the same or identical) genetic background to a modified plant, plant seed, plant part, plant cell, and/or plant genome, if deemed sufficiently similar for comparison of the characteristics or traits to be analyzed.

As used herein, the term “activity” refers to the biological function of a gene or protein. A gene or a protein may provide one or more distinct functions. A reduction, disruption, or alteration in “activity” thus refers to a lowering, reduction, or elimination of one or more functions of a gene or a protein in a plant, plant cell, or plant tissue at one or more stage(s) of plant development, as compared to the activity of the gene or protein in a wild-type or control plant, cell, or tissue at the same stage(s) of plant development. Additionally, an increase in “activity” thus refers to an elevation of one or more functions of a gene or a protein in a plant, plant cell, or plant tissue at one or more stage(s) of plant development, as compared to the activity of the gene or protein in a wild-type or control plant, cell, or tissue at the same stage(s) of plant development.

According to some embodiments, a modified plant is provided having a genomic modification in a target gene that results in reduced, disrupted, or altered activity of the protein encoded by the target gene in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to further embodiments, a modified plant is provided having a protein encoded by a target gene that results in reduced, disrupted, or altered activity in at least one plant tissue by 5%-20%, 5%-25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a control plant.

According to some embodiments, a modified plant is provided having a target mRNA level that is reduced or increased in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to some embodiments, a modified plant is provided having a target mRNA expression level that is reduced or increased in at least one plant tissue by 5%-20%, 5%-25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a control plant. According to some embodiments, a modified plant is provided having a target protein expression level that is reduced or increased in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant. According to some embodiments, a modified plant is provided having a target protein expression level that is reduced or increased in at least one plant tissue by 5%-20%, 5%-25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a control plant.

According to some embodiments, a plant is provided having an gRNA expression level that is reduced or increased in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a control plant.

According to some embodiments, a plant is provided having a recombinant DNA molecule that yields an increase in editing efficiency in at least one plant cell by at least 5%, at least 10%, at least 20%, at least 25%, at least 30%, at least 40%, at least 50%, at least 60%, at least 70%, at least 75%, at least 80%, at least 90%, or 100%, as compared to a control plant.

Modified plants comprising or derived from plant cells that comprise a genome modification of this disclosure can be further enhanced with stacked traits, for example, a modified crop plant having an enhanced trait resulting from expression of DNA disclosed herein in combination with one or more additional genome modifications that provide a beneficial agronomic trait or further improve the enhanced trait.

Modified plants comprising or derived from plant cells that are transformed with a recombinant DNA of this disclosure can be further enhanced with stacked traits, for example, a modified crop plant having an enhanced trait resulting from expression of DNA disclosed herein in combination with one or more genes of agronomic interest that provide a beneficial agronomic trait (such as herbicide and/or pest resistance traits) to crop plants. For example, the traits conferred by the recombinant DNA constructs of the current disclosure can be stacked with other traits of agronomic interest, such as a trait providing insect resistance such as using a gene from Bacillus thuringiensis to provide resistance against lepidopteran, coleopteran, homopteran, hemiopteran, and other insects, or improved quality traits such as improved nutritional value. Molecules and methods for imparting insect/nematode/virus resistance are disclosed in U.S. Pat. Nos. 5,250,515; 5,880,275; 6,506,599; 5,986,175; and U.S. Patent Application Publication No. 2003/0150017 A1.

### VI. Definitions

The following definitions are provided to define and clarify the meaning of these terms in reference to the relevant embodiments of the present disclosure as used herein and to guide those of ordinary skill in the art in understanding the present disclosure. Unless otherwise noted, terms are to be understood according to their conventional meaning and usage in the relevant art, particularly in the field of molecular biology and plant transformation.

When introducing elements of the present disclosure or the embodiment(s) thereof, the articles “a”, “an”, “the”, and “said” are intended to mean that there are one or more of the elements.

The term “and/or”, when used in a list of two or more items, means any one of the items, any combination of the items, or all of the items with which this term is associated.

The terms “comprising”, “including”, and “having” are intended to be inclusive and mean that there may be additional elements other than the listed elements. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

As used herein, a “plant” includes a whole plant, explant, plant part, sicon, seedling, or plantlet at any stage of regeneration or development.

As used herein, a “plant part” can refer to any organ or intact tissue of a plant, such as a meristem, shoot organ/structure (e.g., leaf, stem or node), root, flower or floral organ/structure (e.g., bract, sepal, petal, stamen, carpel, anther and ovule), seed, embryo, endosperm, seed coat, fruit, the mature ovary, propagule, or other plant tissues (e.g., vascular tissue, dermal tissue, ground tissue, and the like), or any portion thereof. Plant parts of the present disclosure can be viable, nonviable, regenerable, and/or non-regenerable. A “propagule” can include any plant part that can grow into an entire plant.

An “embryo” is a part of a plant seed, consisting of precursor tissues (e.g., meristematic tissue) that can develop into all or part of an adult plant. An “embryo” may further include a portion of a plant embryo.

As used herein, “grafting” refers to a vegetative propagation technique that connects two severed plant segments together. For example, the upper part (scion) of one plant grows on the root system (rootstock) of another plant.

A “meristem” or “meristematic tissue” comprises undifferentiated cells or meristematic cells, which are able to differentiate to produce one or more types of plant parts, tissues or structures, such as all or part of a shoot, stem, root, leaf, seed, etc.

As used herein, “virus cleaning” refers to eradication of viral components from a plant. Virus cleaning may be carried out, e.g. using thermotherapy, chemotherapy, nanoparticles and/or in vitro methods.

As used herein, “genomic DNA” or “gDNA” refers to chromosomal DNA of an organism.

As used herein, a “genomic modification” (also referred to as “modification”) or “genomic edit” (also referred to as “edit”) refers to any modification to a genomic nucleotide sequence as compared to a wild-type or control plant. A genomic modification or genomic edit comprises a deletion, an insertion, a substitution, an inversion, a duplication, or any combination thereof.

As used herein, “T-DNA” or “transfer DNA” refers to the transferred DNA of the tumor-inducing (Ti) plasmid of some species of bacteria such as Agrobacterium tumefaciens.

As used herein, an “interfering protein” refers to a protein comprising an alteration that interferes with the normal activity of a protein lacking the alteration, such as a wild-type protein. Non-limiting examples of such interference include, reducing or disrupting the normal protein-protein interactions of the wild-type protein, binding protein-protein interaction partners in a non-functional manner, and/or forming non-functional protein complexes.

As used herein, a “dominant effect” refers to the phenomenon of one allele of a gene on a chromosome masking or overriding the effect of a different allele of the same gene on the other copy of the chromosome. A “dominant effect” may also refer to the observance of an allele associated phenotype in a plant that is heterozygous at the gene of interest.

“Standard agronomic practices” are known to those of skill in the art and refer to well-accepted methods and techniques for the cultivation and evaluation of crop species. For example, a field trial carried out under standard agronomic practices carefully controls for confounding factors including the environment, and thus the trial reflects the intrinsic morphology and physiology of the varieties being tested.

As used herein, the “vegetative phase” of plant development is the period of growth between germination and flowering. For example, in maize, a common plant development scale used in the art is known as V-Stages. The V-stages are defined according to the uppermost leaf in which the leaf collar is visible. VE corresponds to emergence, V1 corresponds to first leaf, V2 corresponds to second leaf, V3 corresponds to third leaf, V(n) corresponds to nth leaf. VT occurs when the last branch of tassel is visible but before silks emerge. When staging a field of maize, each specific V-stage is defined only when 50 percent or more of the plants in the field are in or beyond that stage. Other development scales are known to those of skill in the art and may be used with the methods of the invention. The stages in the reproductive phase of corn are as follows R1 (silking; silks emerge from husks); R2 (blister; kernels are white on outside and inner fluid is clear); R3 (milk, kernels are yellow on the outside and inner fluid is milky-white); R4 (dough; milky inner fluid thickens from starch accumulation); R5 (dent; more than 50% of kernels are dented); and R6 (physiological maturity; black layer formed). Vegetative and reproductive stages such as those described herein for corn are well known to those of skill in the art and numerous publications describing these stages can be found on the world wide web and elsewhere.

As used herein, the term “isogenic” means genetically uniform, whereas non-isogenic means genetically distinct.

All methods described herein can be performed in any suitable order unless otherwise indicated herein or clearly contradicted by context. The use of any and all examples, or exemplary language (e.g., “such as”) provided with respect to certain embodiments herein is intended merely to illuminate the present disclosure and does not pose a limitation on the scope of the present disclosure otherwise claimed.

### EXAMPLES

### Example 1. Generation of Plant Transformation Vectors

TRV RNA1 (Vector YL192-pTRV1) and TRV RNA2 (Vector YL156-pTRV2-MCS), are used for transient virus-induced gene silencing. These vectors were modified to enable the stable transformation of N. tabacum.

In brief, the vector pTRV1 was digested with SmaI and PmeI restriction digestion enzymes to remove the bar selectable marker gene. The digested vector was self-ligated to generate pFGR15, which was digested with BamHI and treated with Mung bean nuclease (MBN) (New England Biolabs®) to remove 5′ overhang ends using 1 μL of MBN, 1 μg of digested plasmid and 1×MBN buffer and self-ligated. Subsequently, pFGR15 was digested with SbfI to clone the NptII gene, which was amplified from the pCambia 2300 plasmid using Q5 Hi-fidelity Taq polymerase (New England Biolabs®) as per manufcaturer's protocol with primers FGRP3 and FGRP4. The PCR product was cleaned using a QIAquick PCR purification kit (Qiagen®), digested with SbfI enzyme and ligated into SbfI digested pFGR15 to generate plant transformation vector pFGR27 with NptII as the plant selectable marker.

The pCambia2300 vector was digested with EcoRI and SmaI restriction digestion enzymes to remove the multiple cloning site. The EcoRI staggered end was treated with MBN to generate a linearized plasmid with blunt ends. The resulting plasmid was self-ligated using T4 DNA ligase. The TRV RNA2 sequence was PCR amplified with Q5 Long Amp polymerase (New England Biolabs®) using primers FGRP165 and FGRP166. The PCR reaction was cleaned using QIAquick PCR purification kit, and digested with HindIII and PmeI and ligated in pCambia2300 digested with the same enzymes to generate plasmid pFGR28.

To obtain the gRNA targeting the NtPDS gene, plasmid pKSE401 was digested with BsaI, which generates staggered ends. The NtPDS gRNA was generated by annealing 100 μM each of the primers BWBP268 and BWBP269 with the following program: 10 minutes at 95° C., decreasing temperature by −1° C./minute till the temperature reached 25° C. When annealed, the primers generated four nucleotide overhangs at 5′ and 3′ ends compatible with BsaI. The annealed primers were ligated into pKSE401 using 1:6 pg (vector:insert) 0.5 μL T4 DNA ligase, and 1×T4 DNA ligase buffer (New England Biolabs®) generating the plasmid pVIG12.

To obtain the Cas9 gene as an insert, the plasmid pKSE401 was first digested with HindIII and self-ligated to remove gRNA promoter and scaffold sequences and then further digested with EcoRI and PmeI. The Cas9 insert was ligated into the multiple cloning site of pFGR28 digested with the same enzymes (EcoRI and PmeI) generating the plasmid pFGR29.

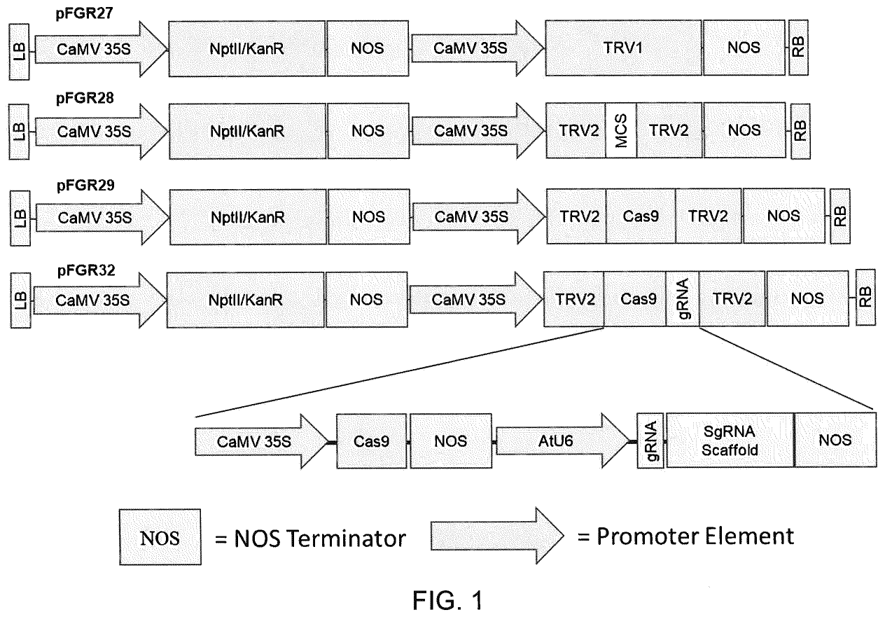

Thereafter, pVIG12 plasmid was digested with EcoRI and PmeI restriction digestion enzymes to obtain the insert representing gRNA sequence, driven by AtU6 promoter along with the Cas9 gene driven by the CaMV35S promoter, and ligated into pFGR28 digested EcoRI and SmaI generating the plant transformation vector pFGR32 (Table 1 and FIG. 1).

TABLE 1
Summary of Plant Transformation Vectors

| | Plasmid | |
|---|---|---|
| SEQ ID NO | Identifier | Description |
| SEQ ID NO: 1 | pFGR32 | pCambia 2300 with TRV RNA2, Cas9, |
| | | NtPDS gRNA, NptII as a selectable |
| | | marker for plant transformation |
| SEQ ID NO: 2 | pFGR29 | pCambia 2300 with TRV RNA2 and Cas9, |
| | | NptII as a selectable marker for plant |
| | | transformation |
| SEQ ID NO: 4 | pFGR28 | pCambia 2300 with TRV RNA2, NptII as a |
| | | selectable marker for plant transformation |
| SEQ ID NO: 3 | pFGR27 | YL 156 with TRV RNA1, NptII as a |
| | | selectable marker for plant transformation |
| SEQ ID NO: 5 | pFGR15 | YL 156 with TRV RNA1, bar gene removed |
| SEQ ID NO: 6 | pVIG12 | pKSE401 with Cas9 and gRNA to target |
| | | NtPDS gene in N. tabacum |

### Example 2. N. tabacum Growth, Propagation, and Transformation

N. tabacum Plants were propagated in MS media (Murashige et al., Physiol. Plant. 1962) as described previously (Dhingra et al., Proc. Natl. Acad. Sci. U.S.A 2004) and maintained at 24±1° C. with a 16/8 h light/dark photoperiod. Leaf regeneration was performed on MS media supplemented with 1.0 mg/L BAP, 0.2 mg/L NAA, 0.7% Agar, pH 5.8 with or without Kanamycin (100 mg/L) as needed.

Agrobacterium strain GV3101 harboring all plant transformation vectors were used for co-cultivation with leaf explants of N. tabacum cv. Petit Havana plants. Leaf discs were prepared for co-cultivation using a sterile cork borer (8 mm). The explants were placed adaxial side up onto the MS medium and incubated at 25° C. under an 18-h-light/6-h-dark cycle for 24 hours prior to co-cultivation treatments described in Table 2. Agrobacterium suspension was diluted in MS media to OD₆₀₀ 0.8 with 200 μM Acetosyringone for 10 minutes under vacuum and then incubated at ambient temperature for 30 minutes.

Leaf discs were then placed on solid co-cultivation media (MS Media, 200 μM Acetosyringone, 1.0 mg/L BAP, 0.1 mg/L NAA, 0.7% Agar, pH 5.8) and incubated at 25° C. in the dark for 48 hours. The leaf discs were moved to selection media MS media supplemented with 1.0 mg/L BAP, 0.2 mg/L NAA, 0.7% Agar, 200 mg/L Timentine, 200 mg/L Kanamycin, and pH 5.8. Potential transgenic shoots were transferred to the rooting media MS media with 2% sucrose, 0.7% Agar, 200 mg/L Timentine, 200 mg/L Kanamycin and pH 5.8.

TABLE 2
Summary of N. tabacum transformation

| Treatment | Description |
|---|---|
| T1 | Regeneration control - no selection |
| T2 | Negative regeneration control |
| T3 | pCambia 2300 |
| T4 | pFGR27 + 28 |
| T5 | pFGR27 + 29 |
| T6 | pFGR27 + 32 |
| T7 | pVIG12 |

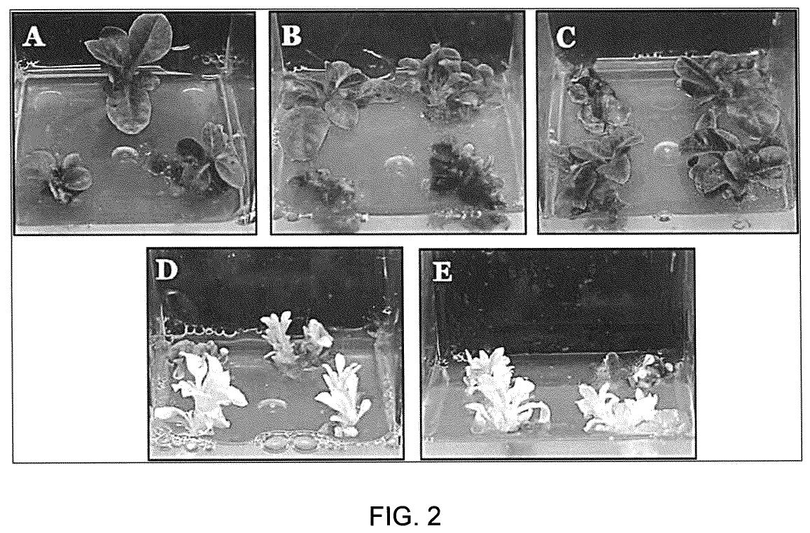

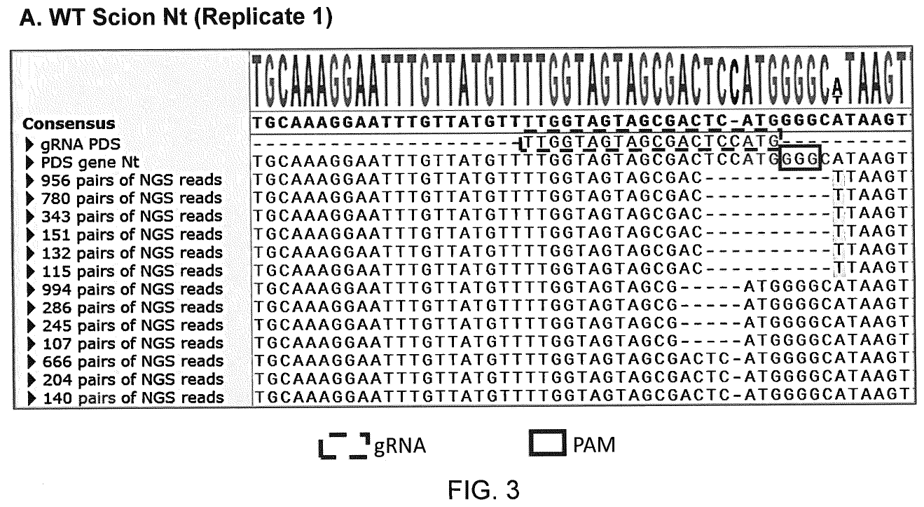

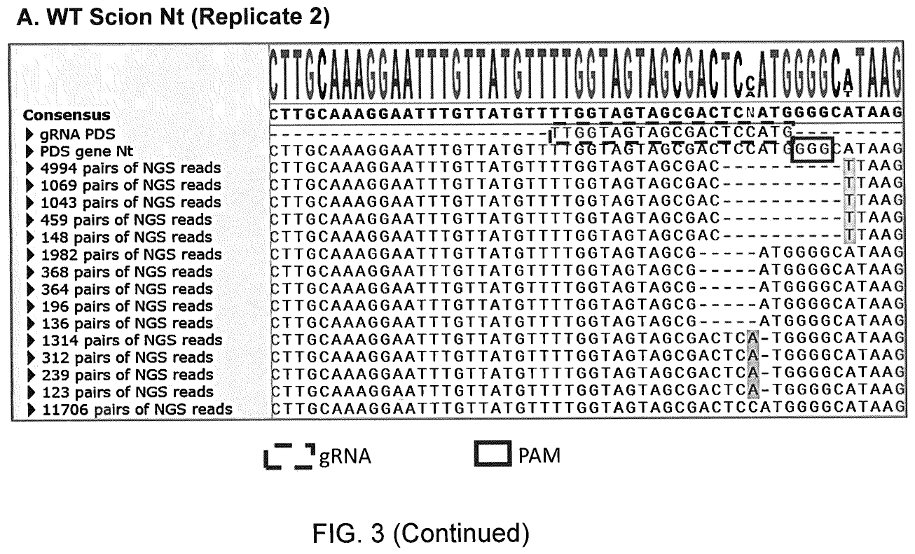

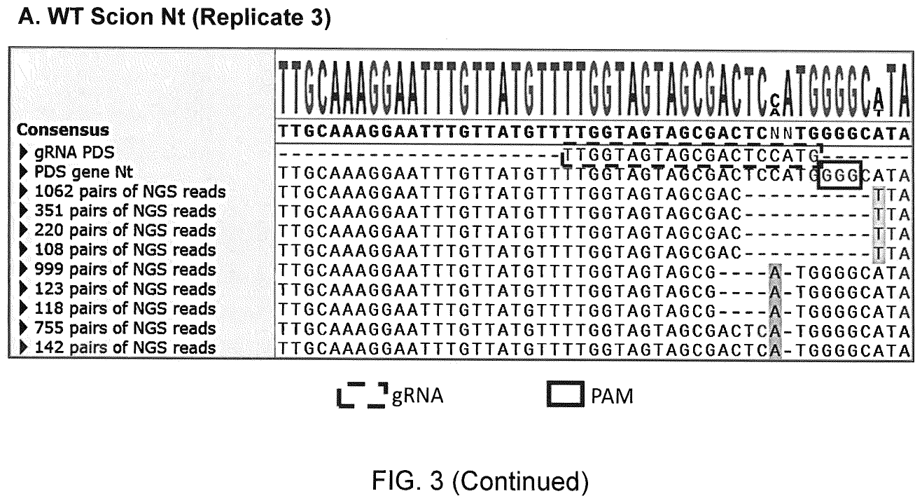

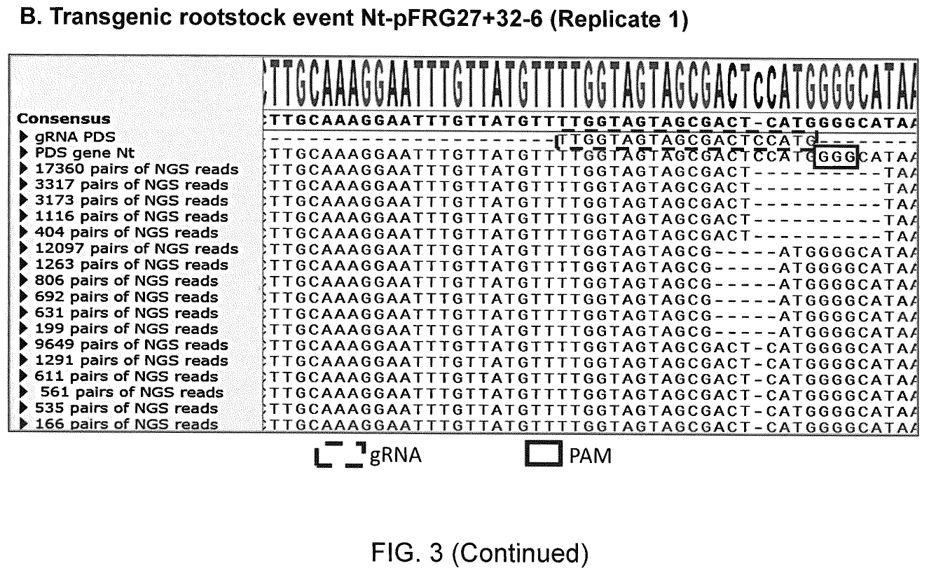

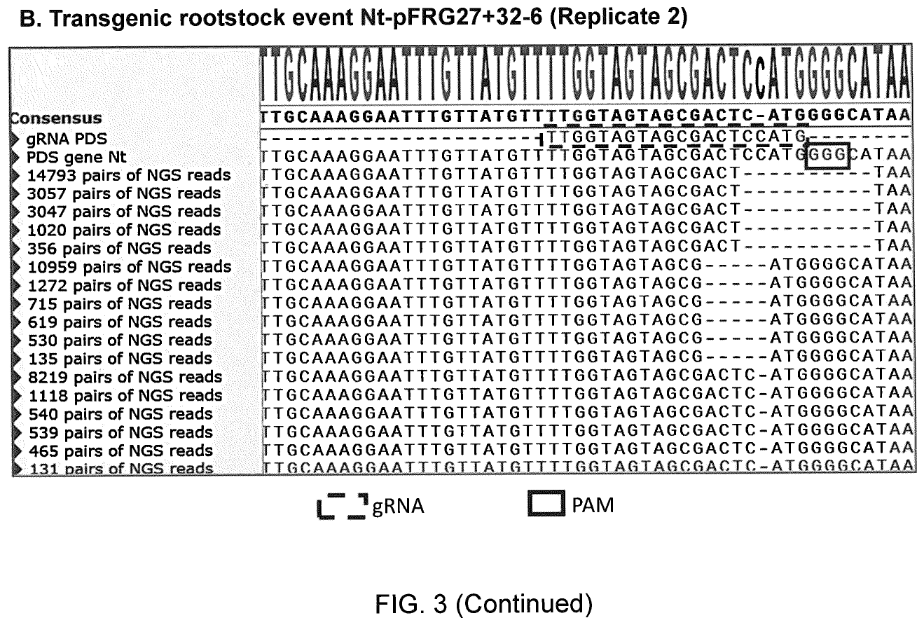

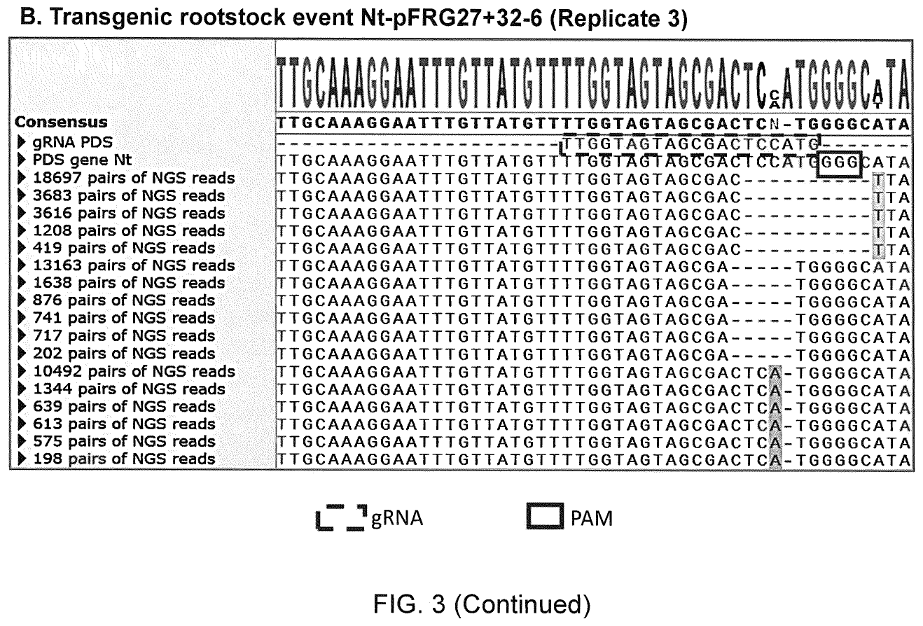

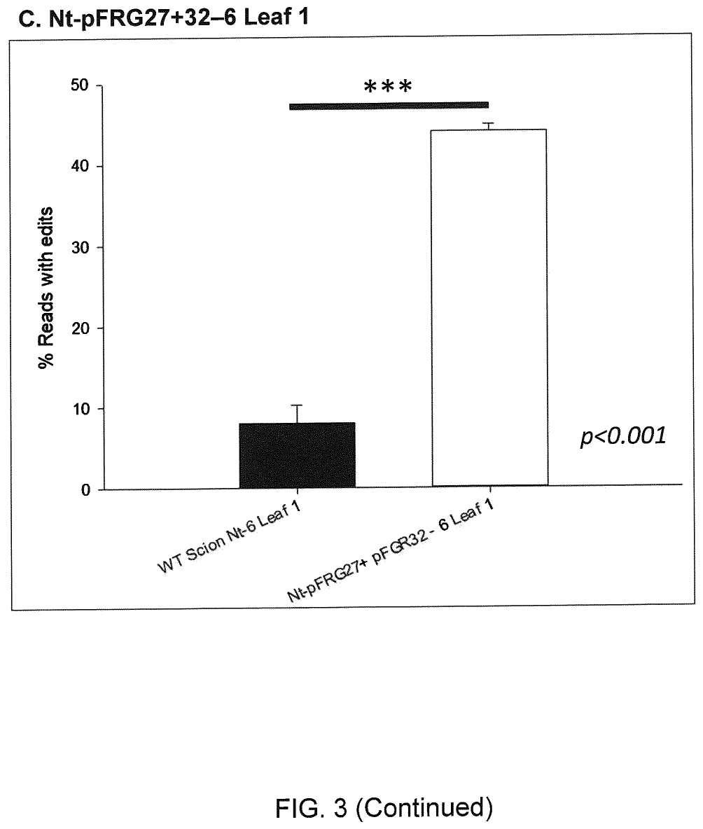

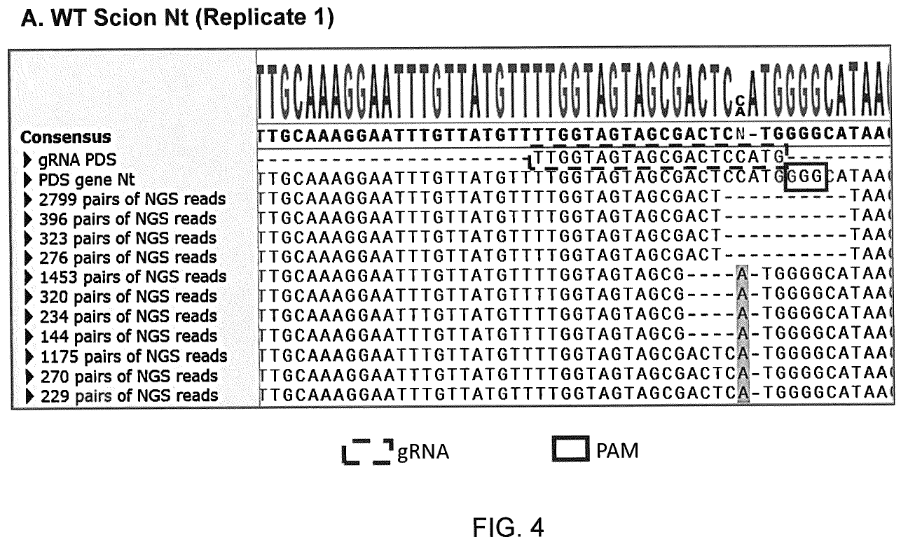

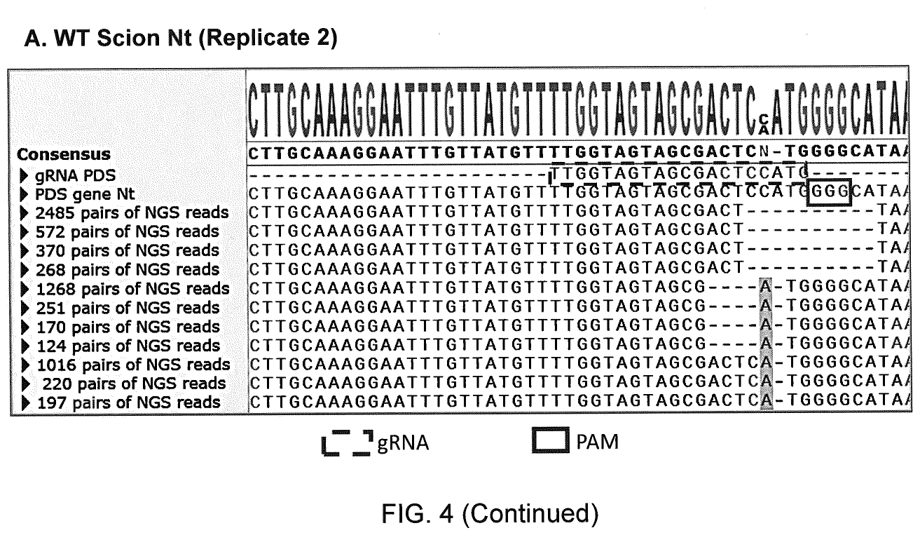

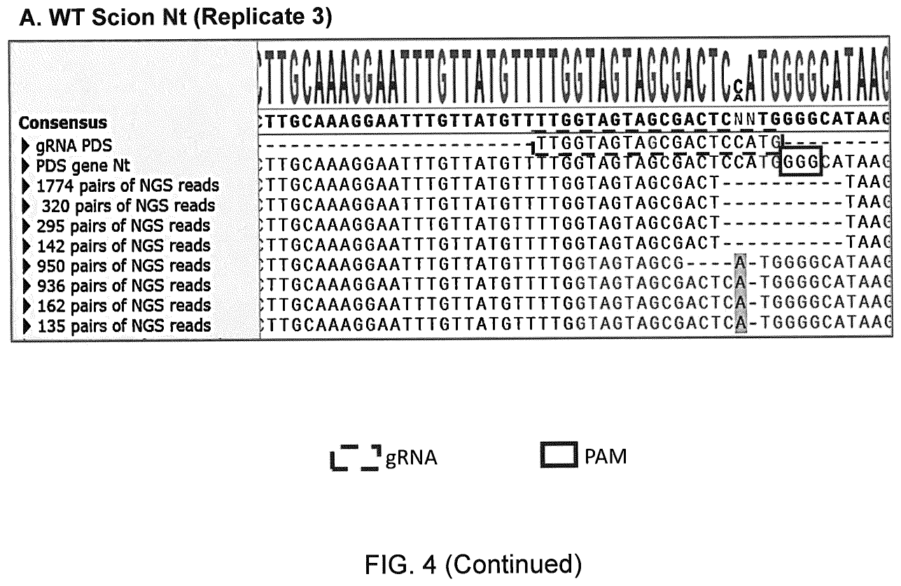

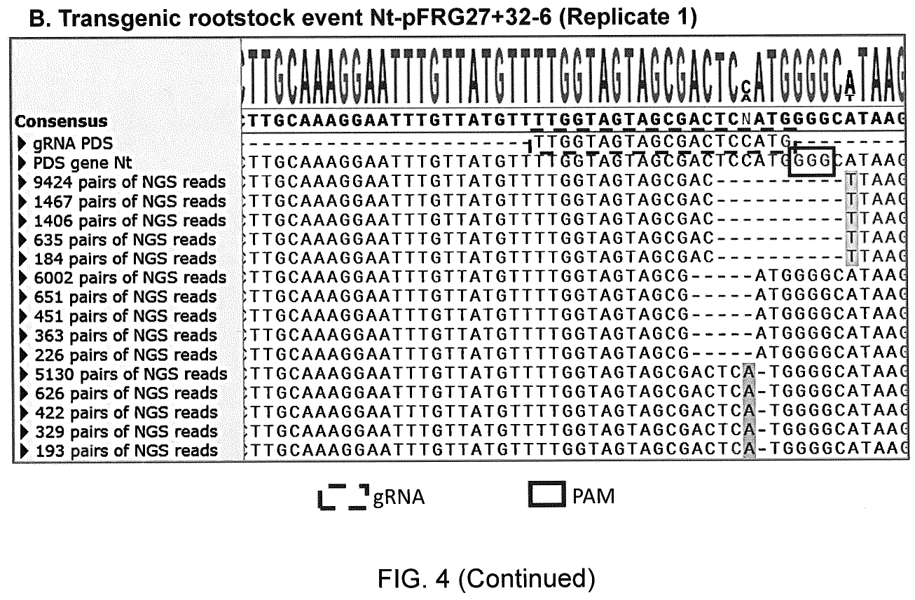

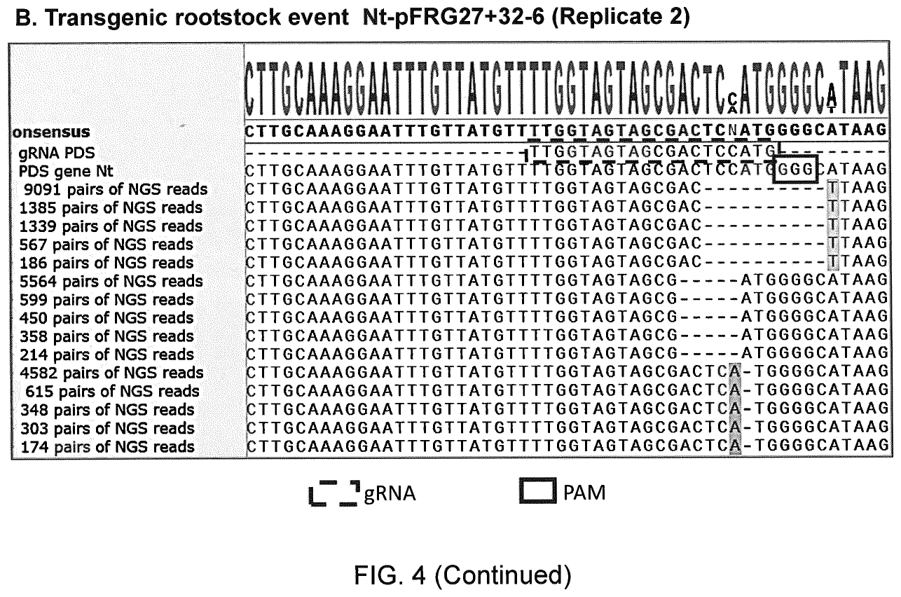

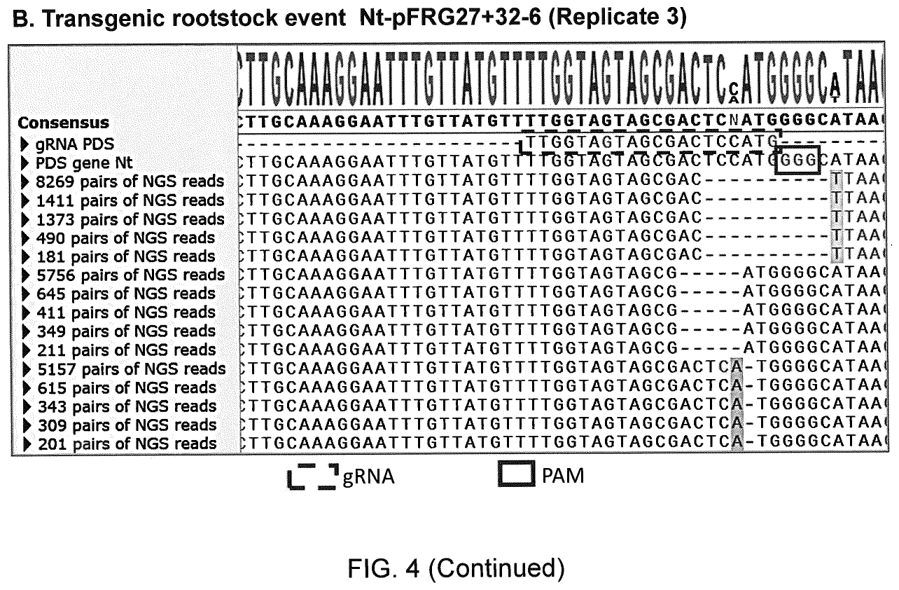

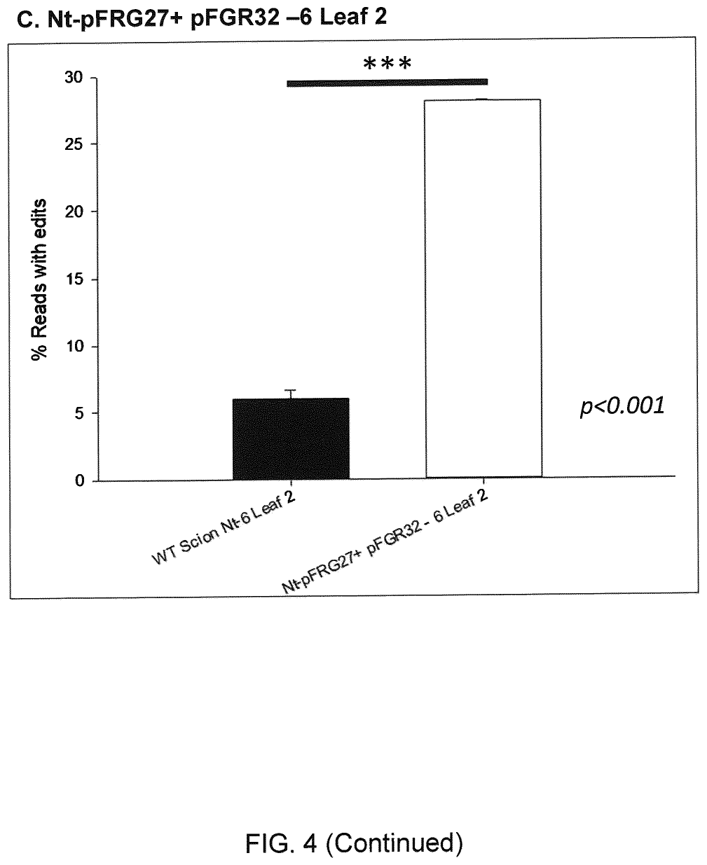

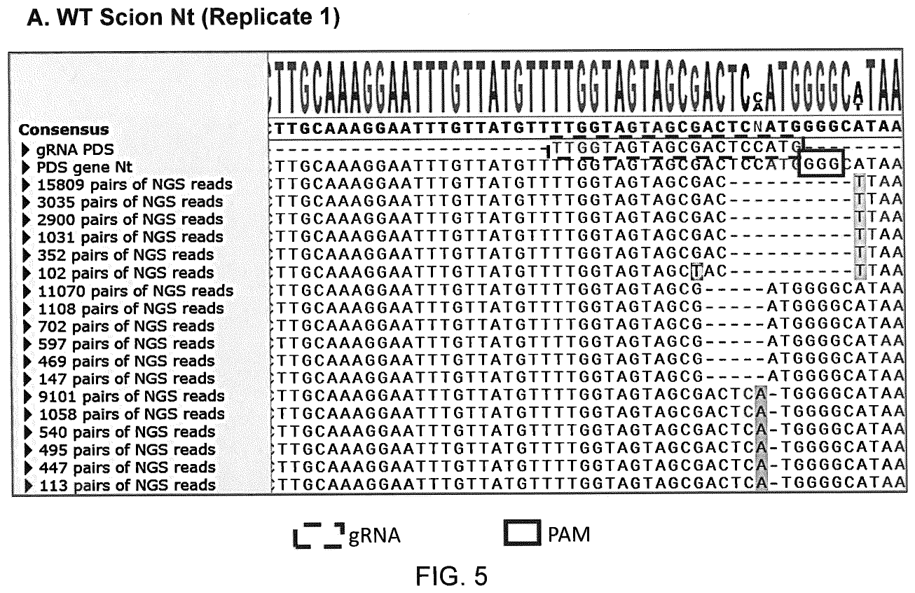

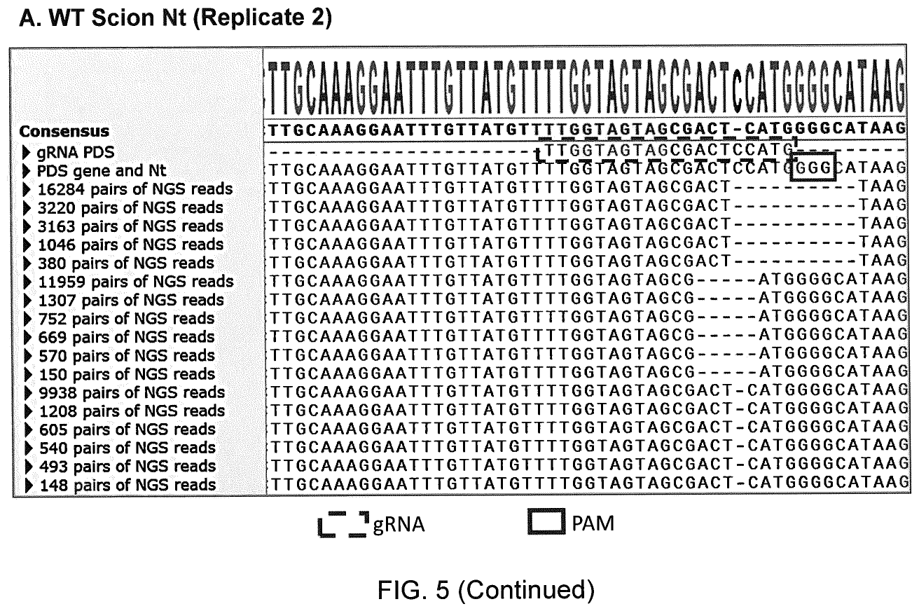

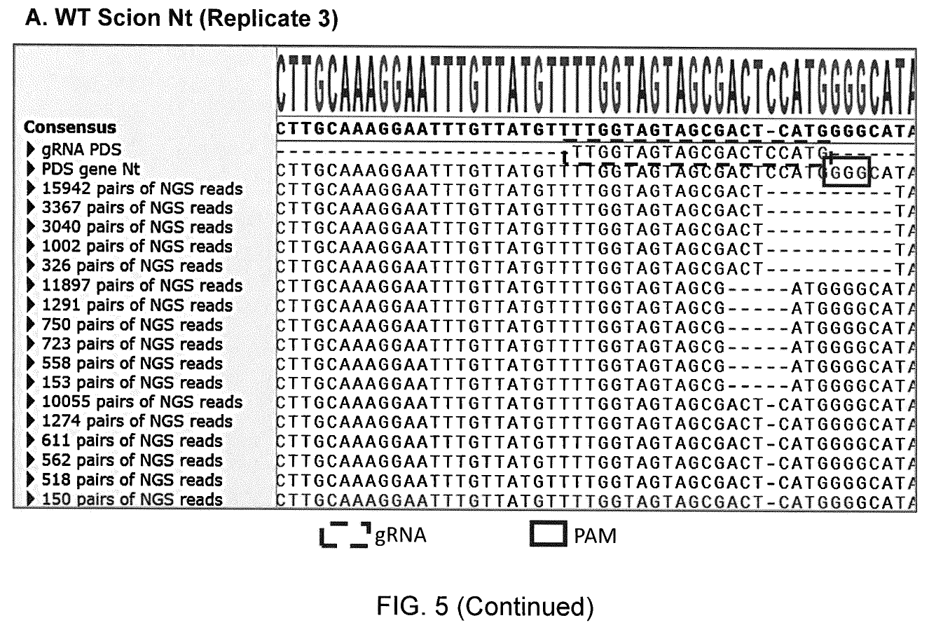

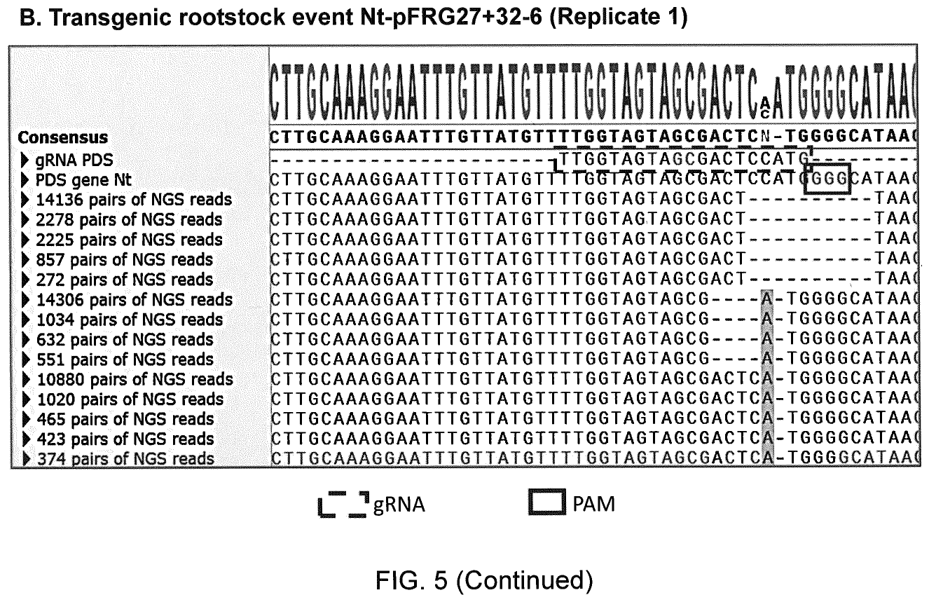

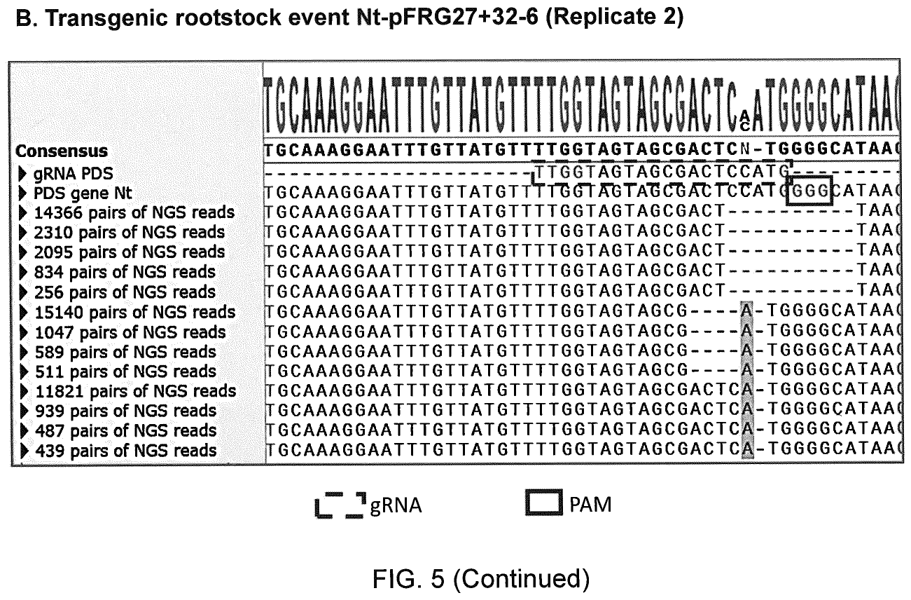

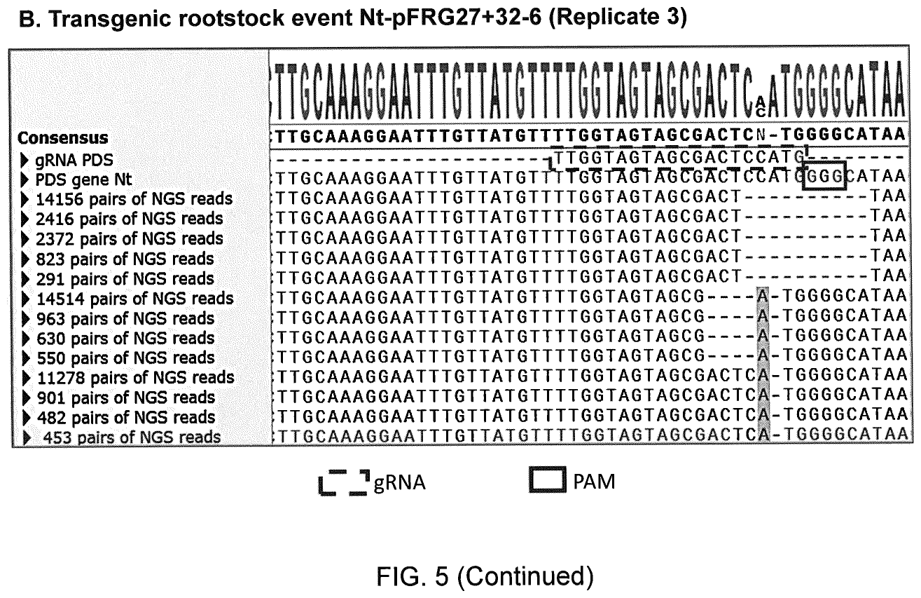

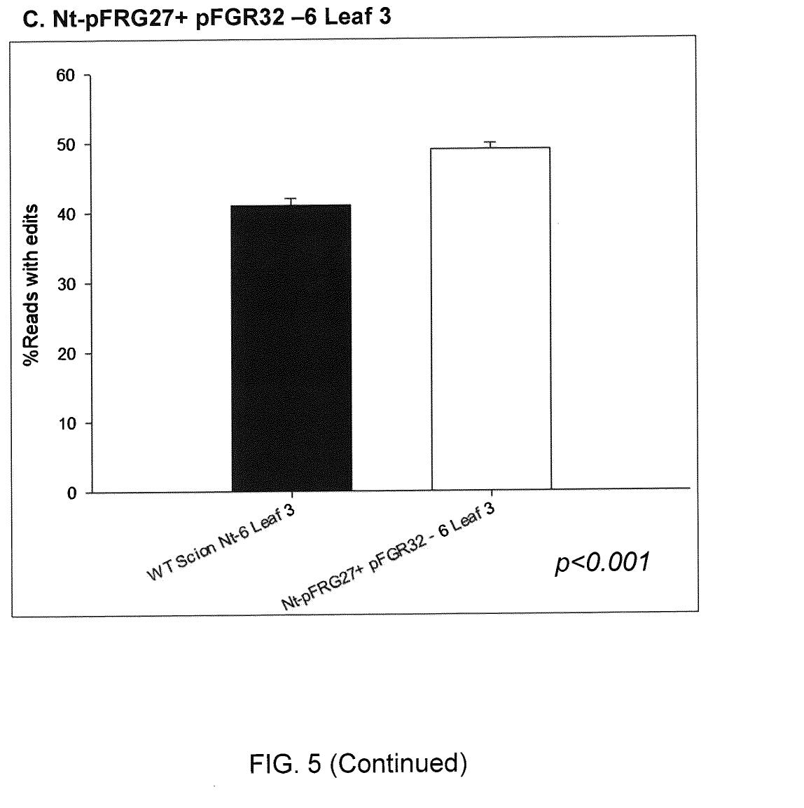

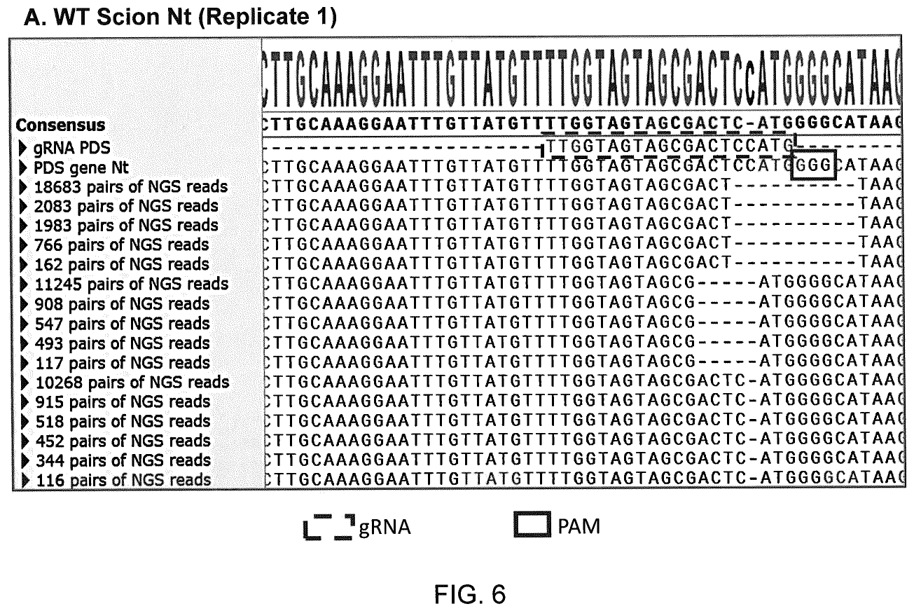

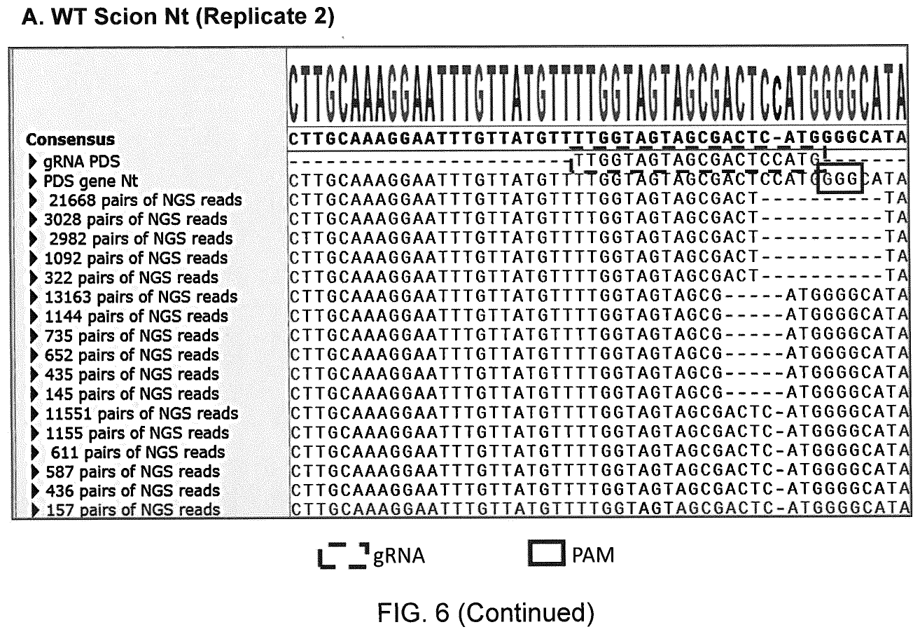

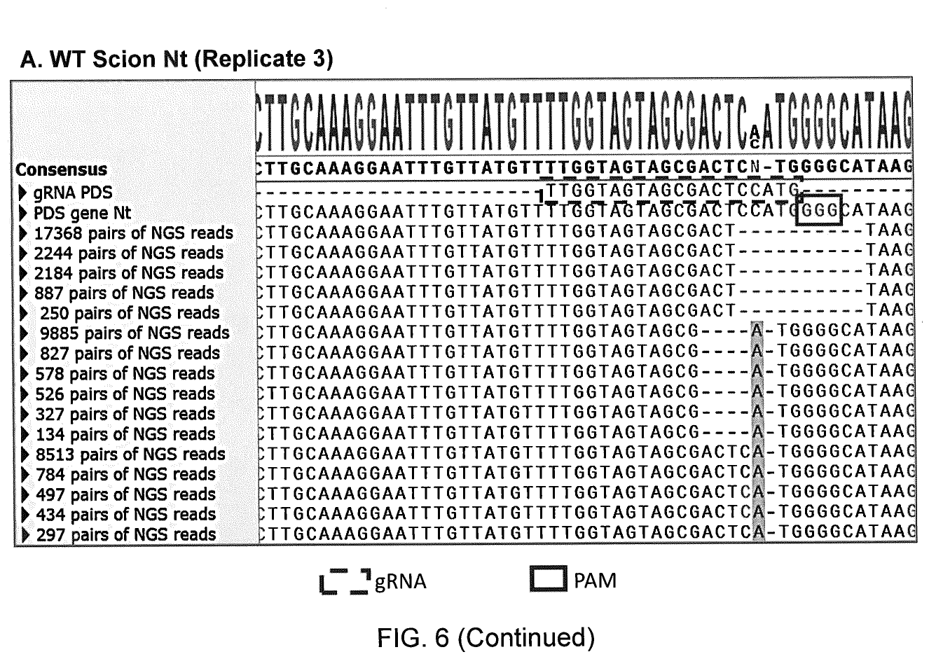

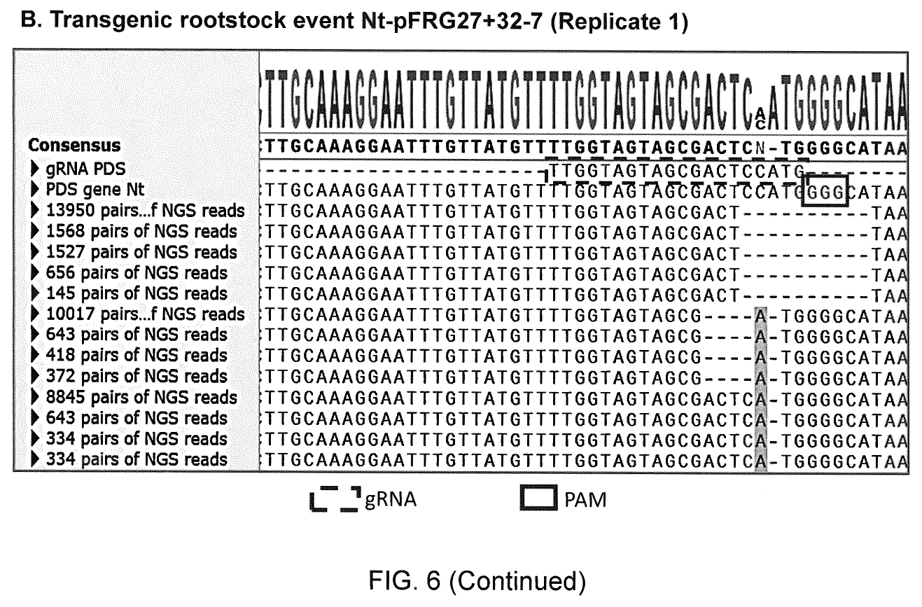

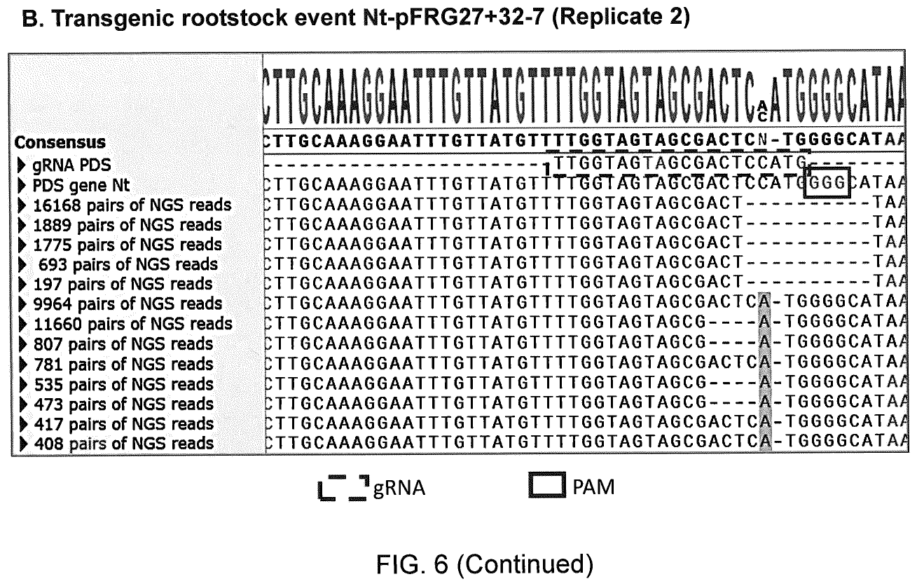

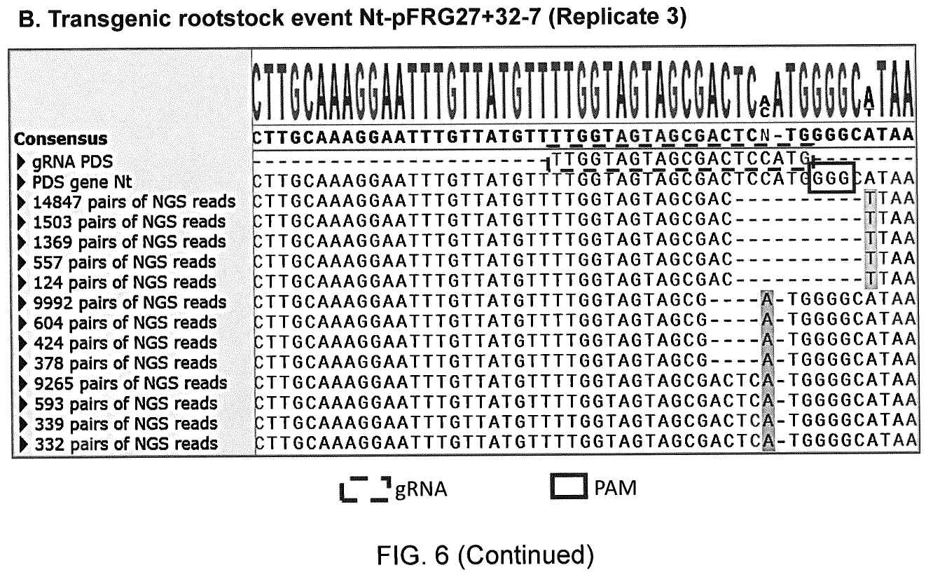

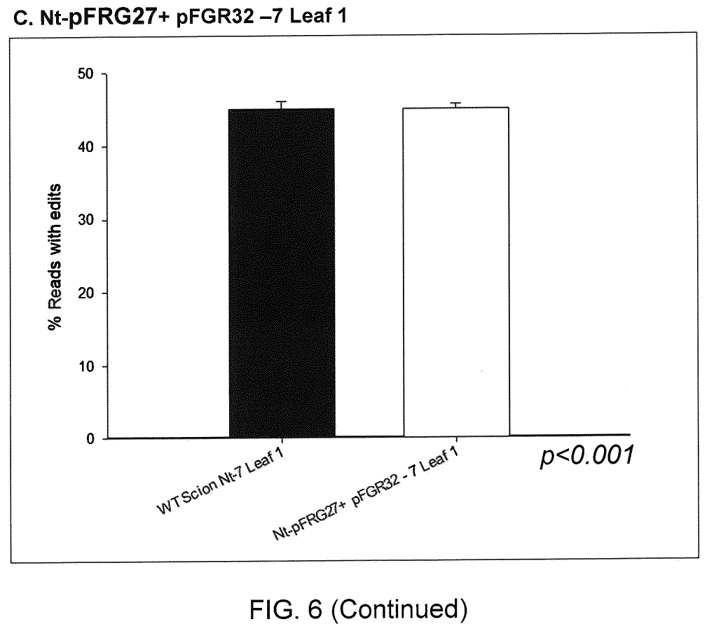

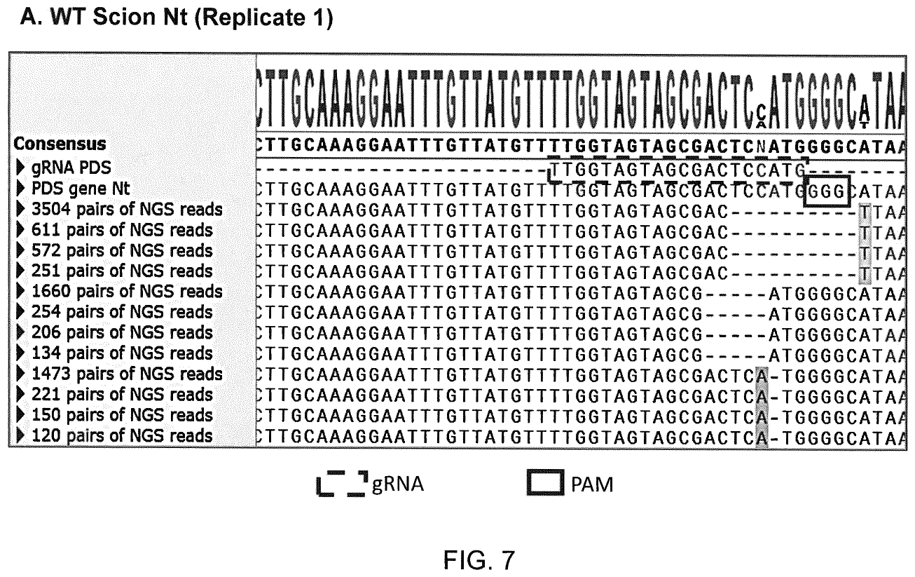

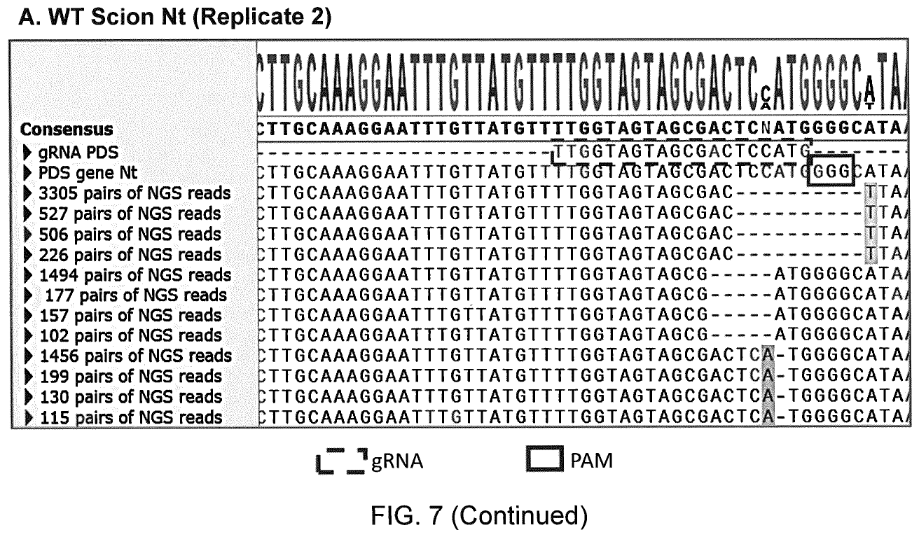

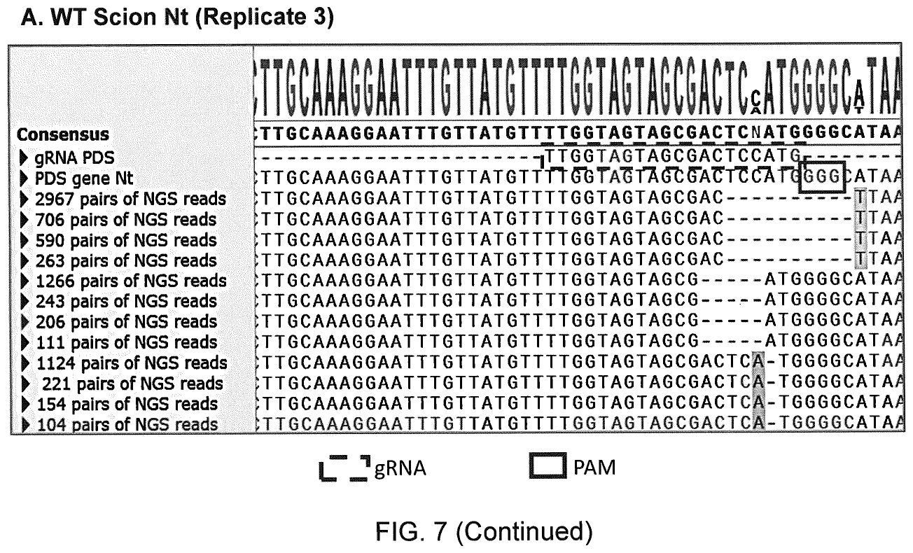

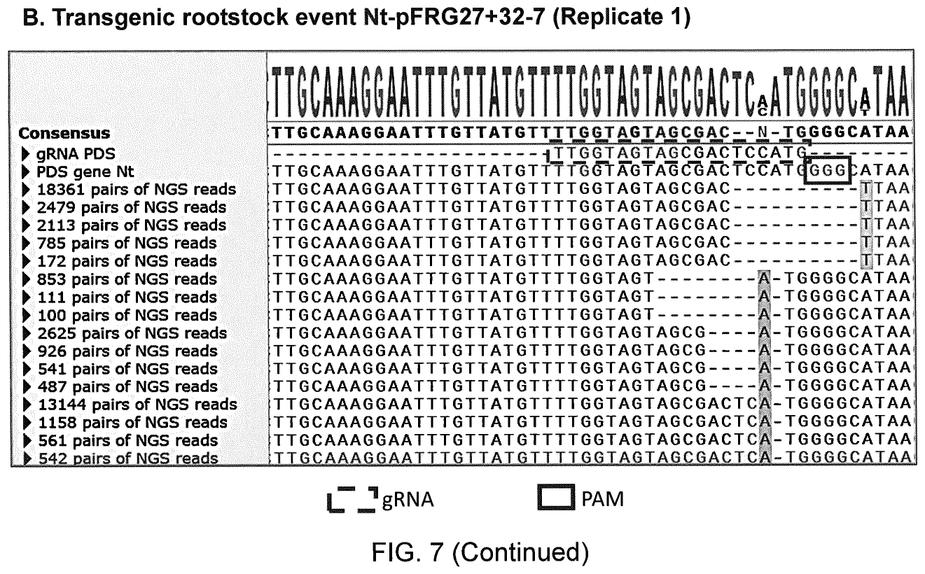

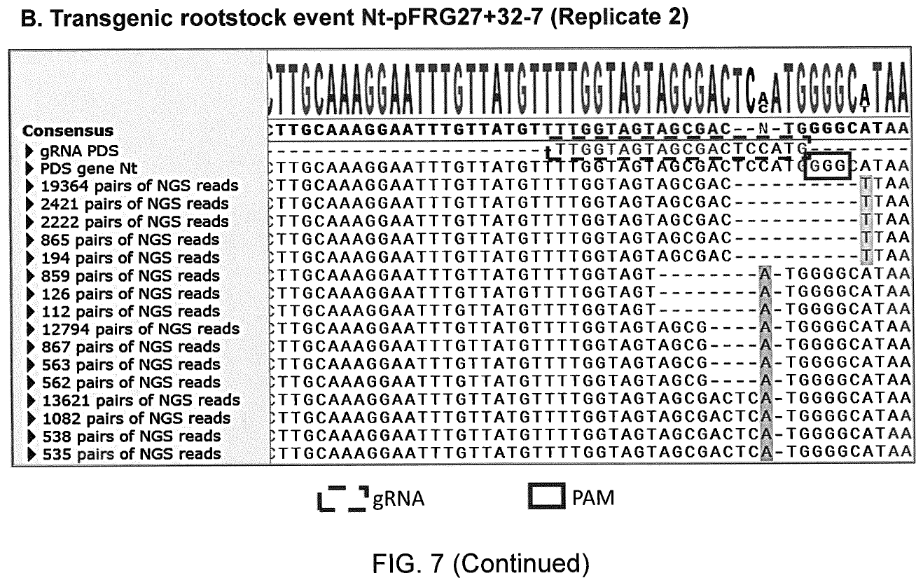

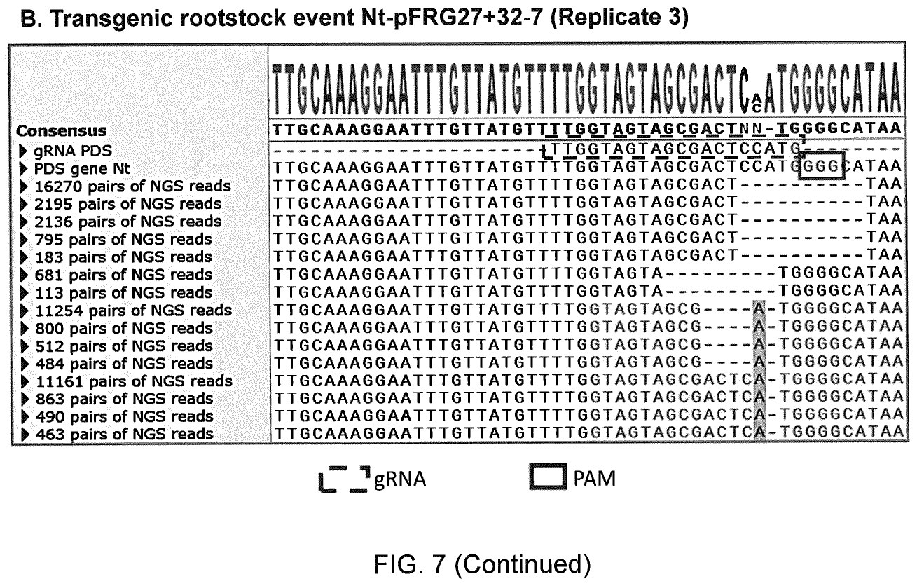

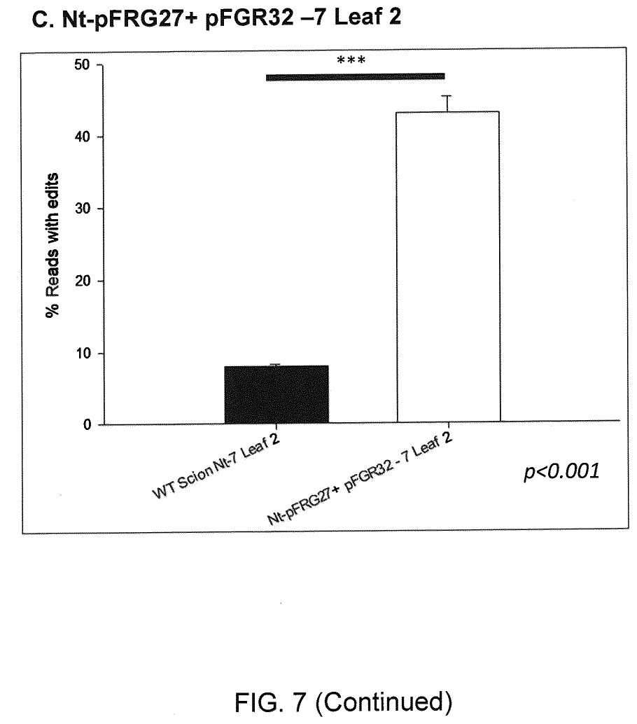

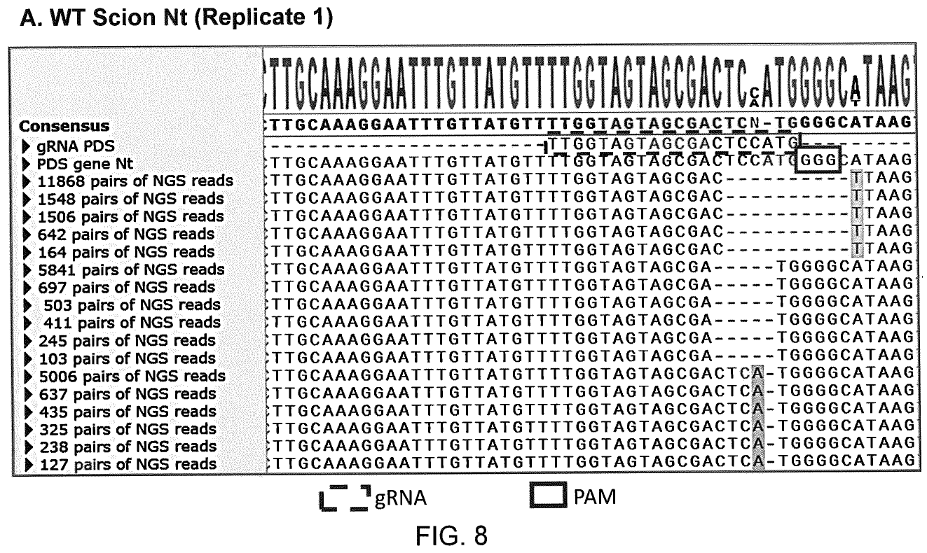

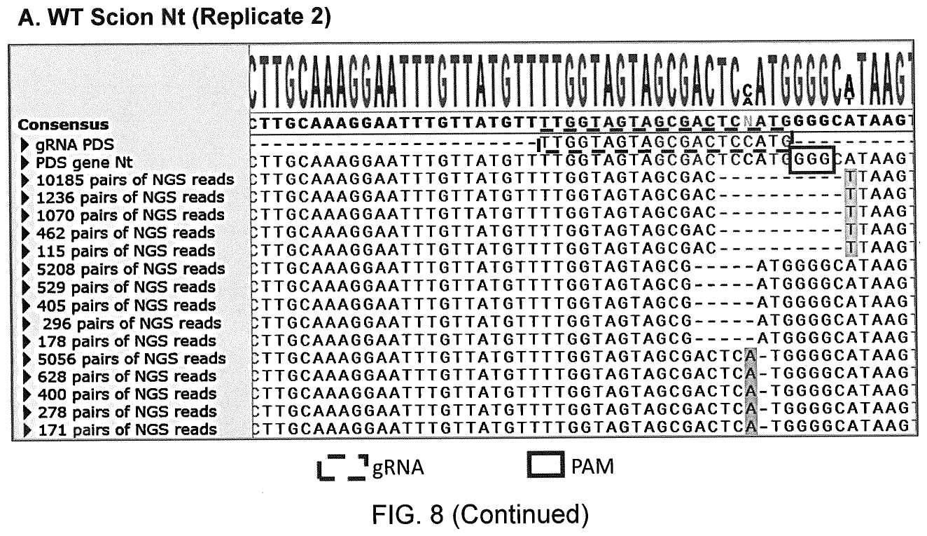

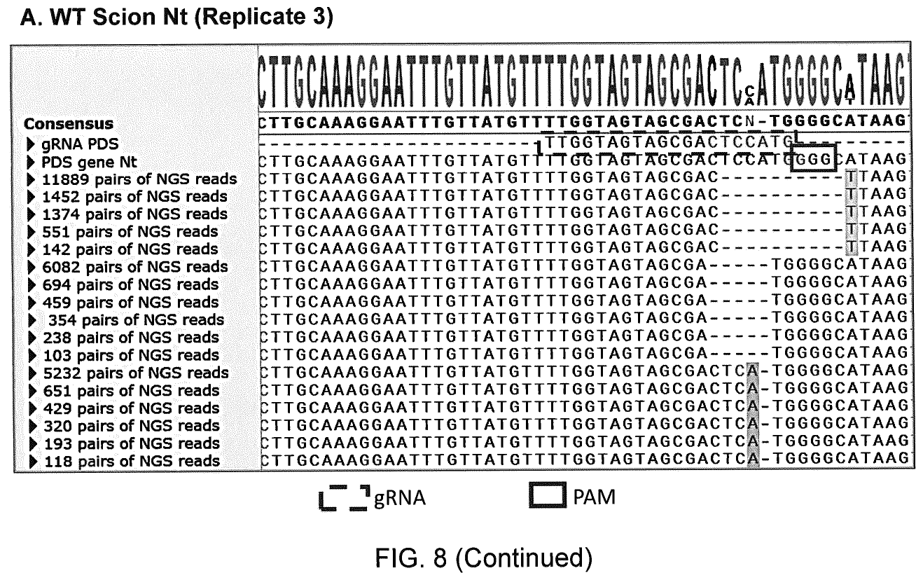

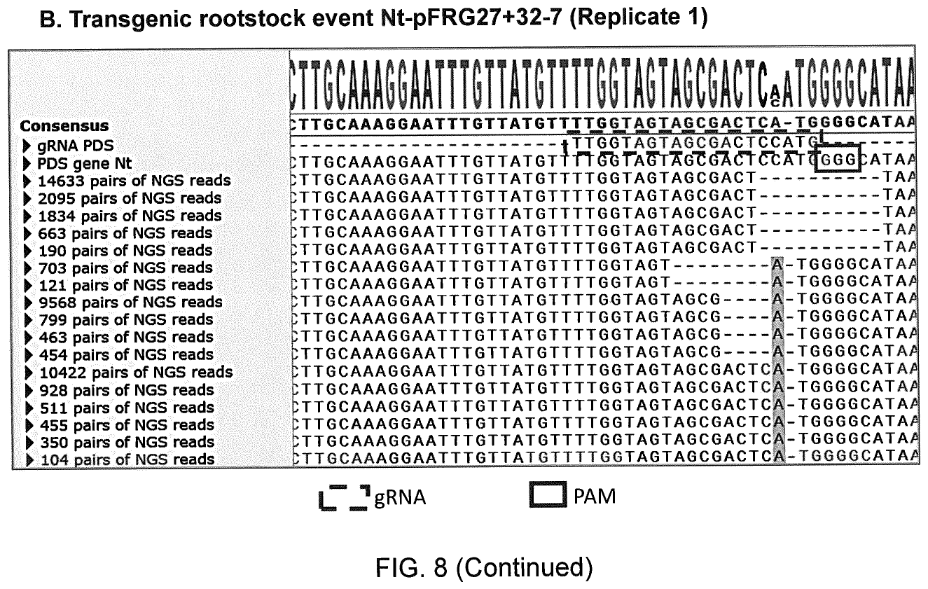

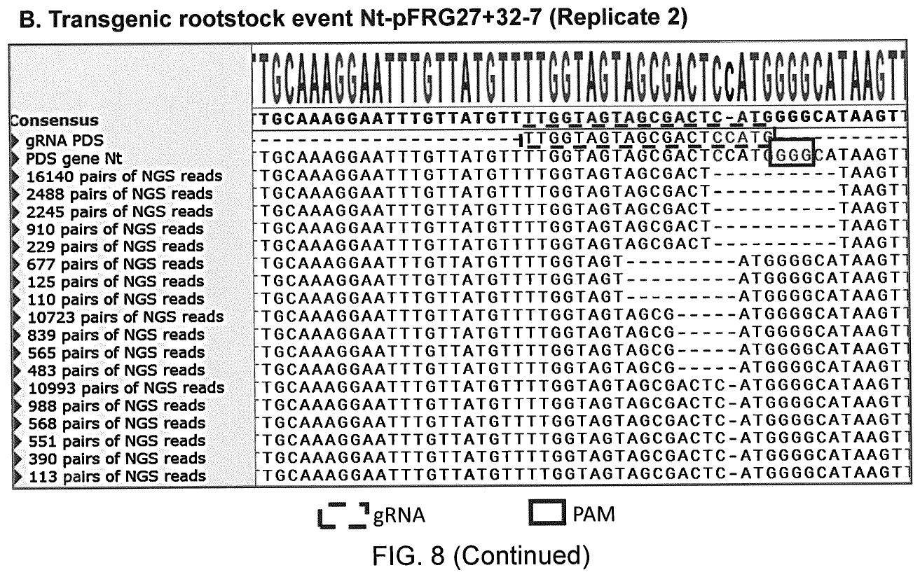

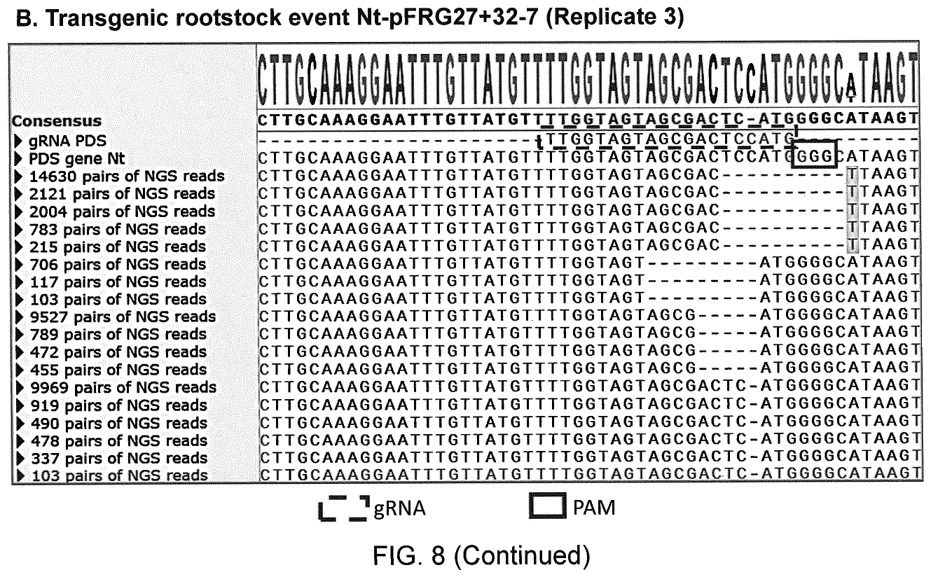

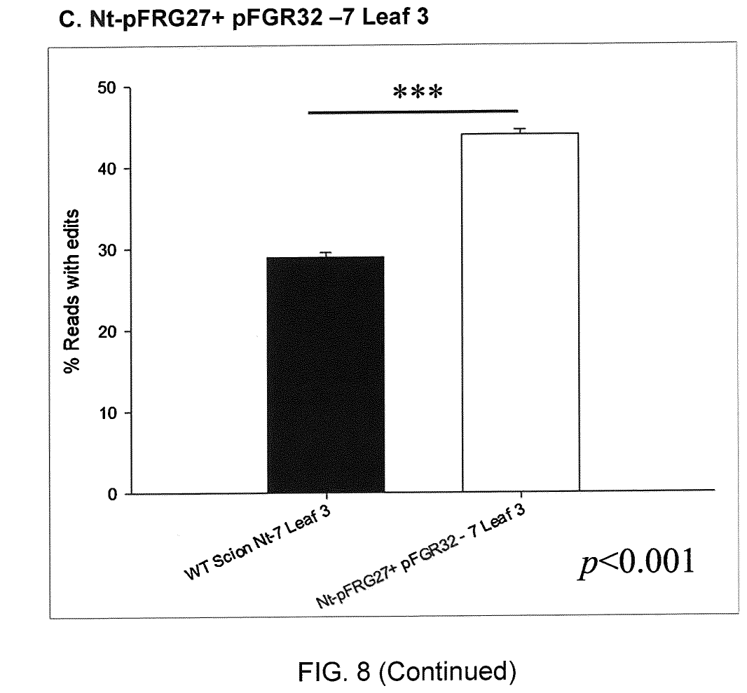

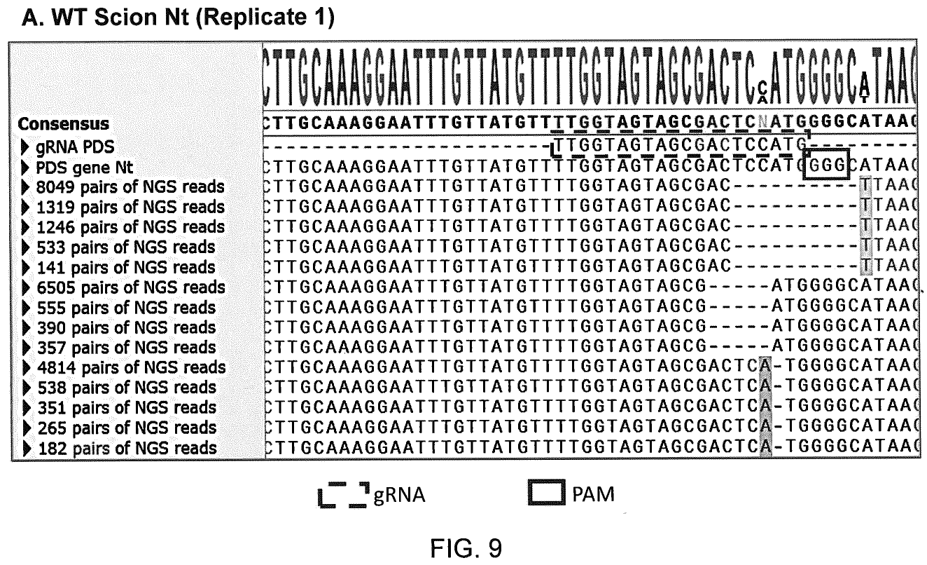

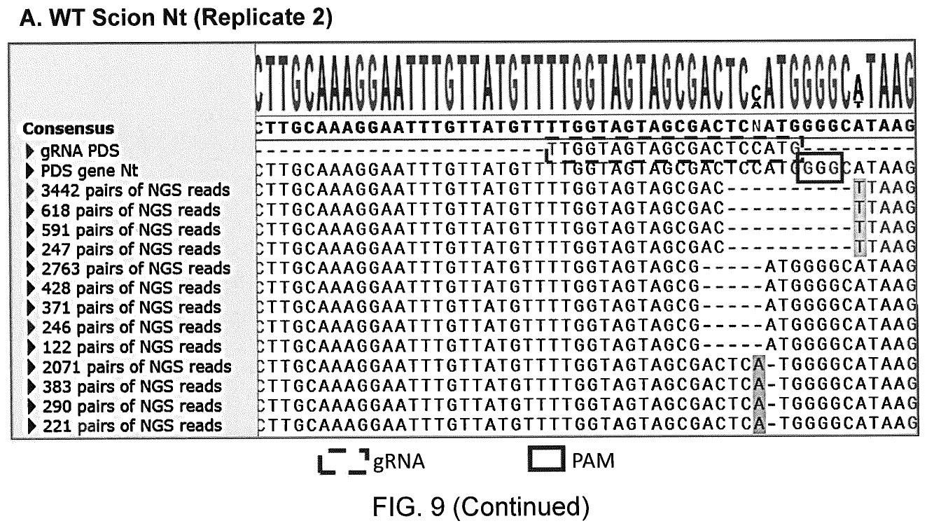

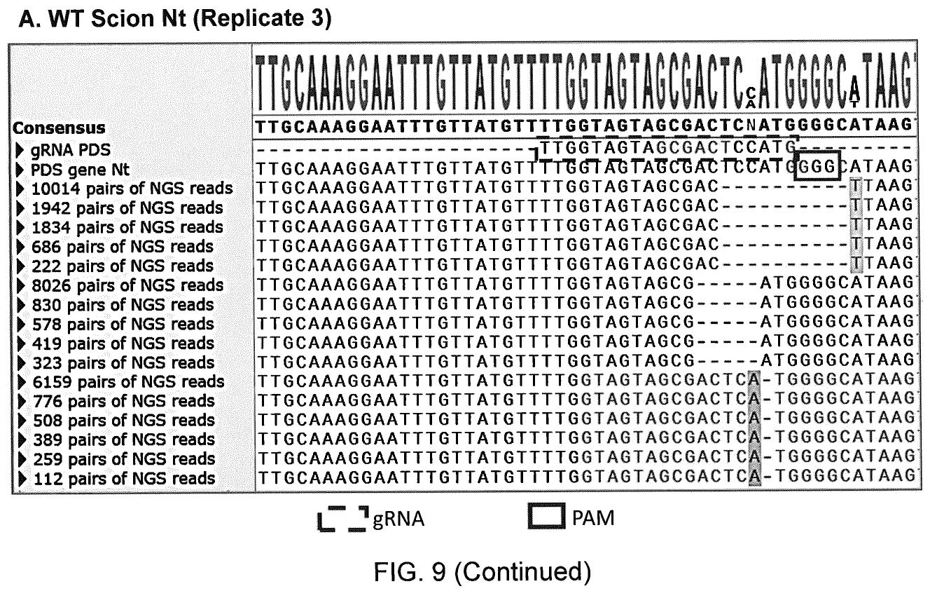

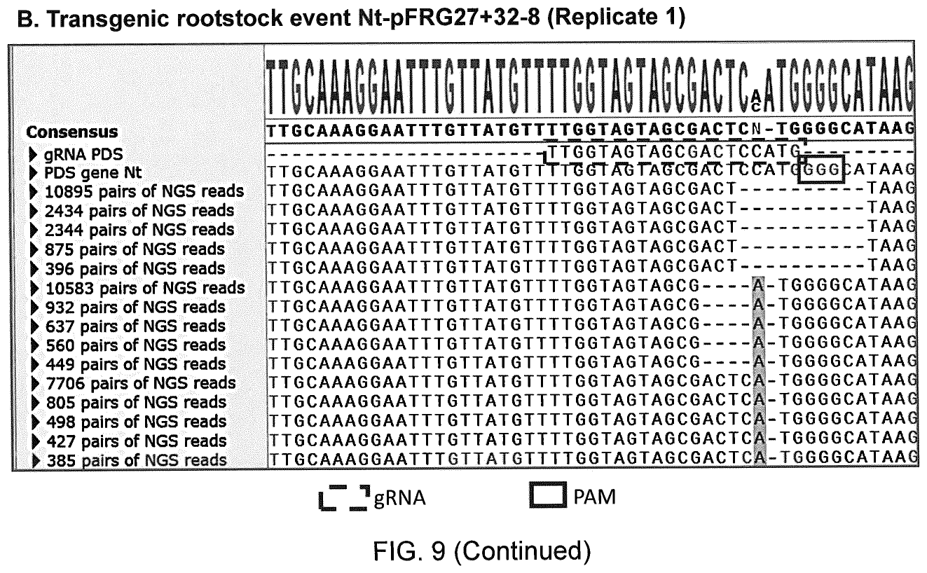

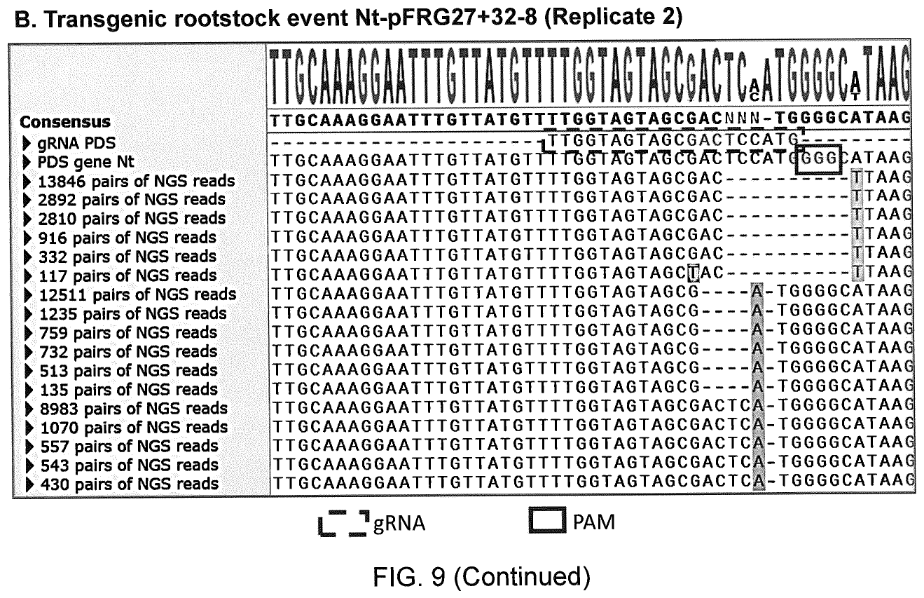

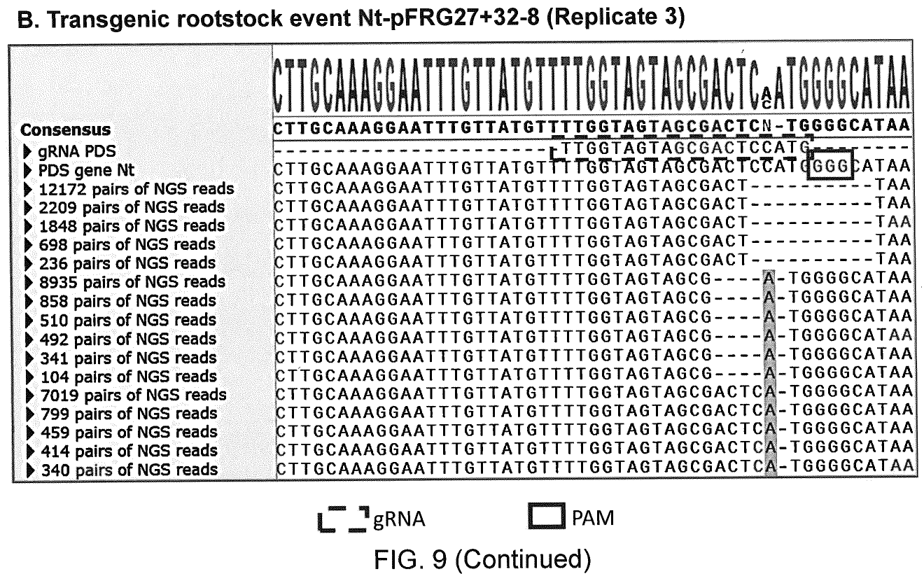

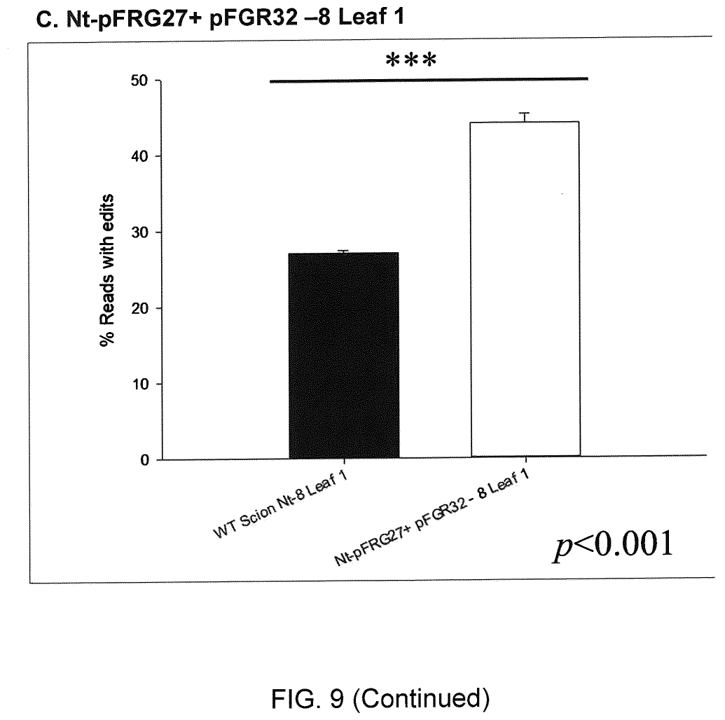

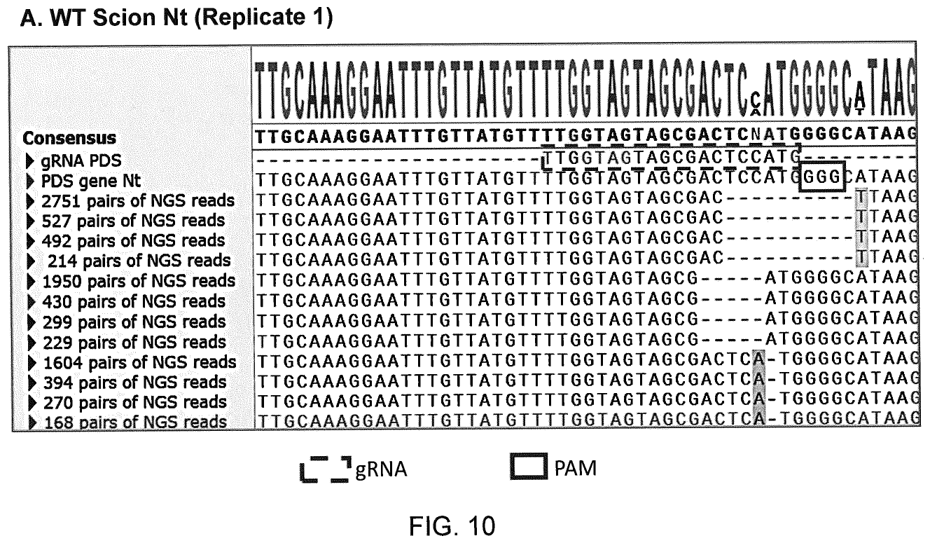

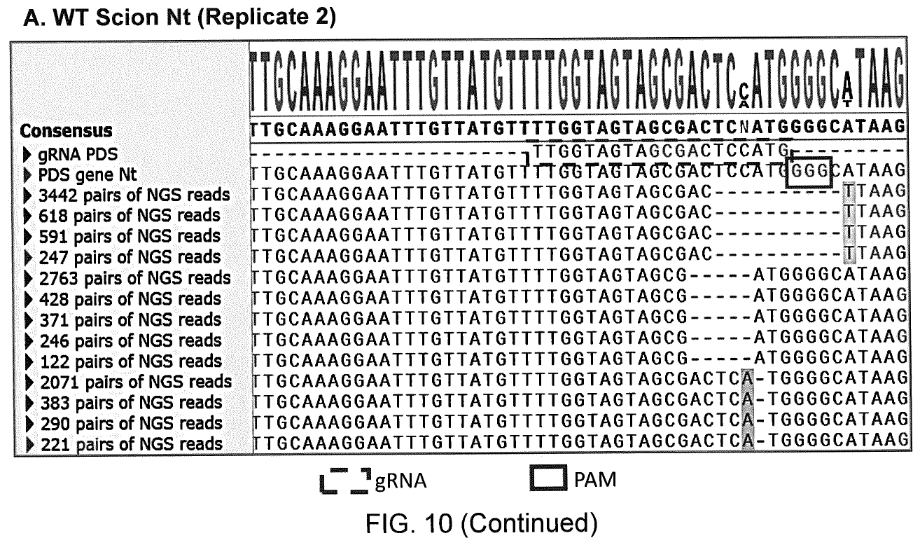

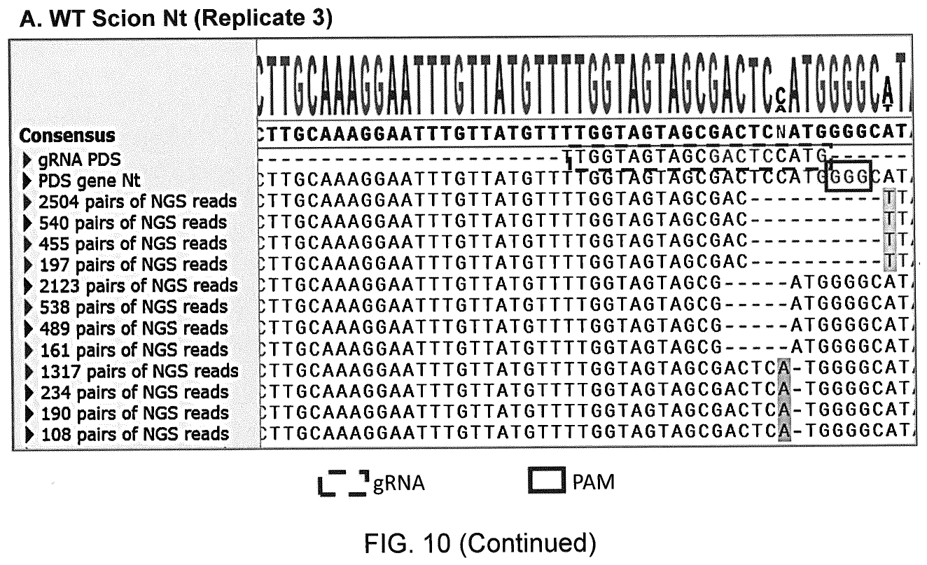

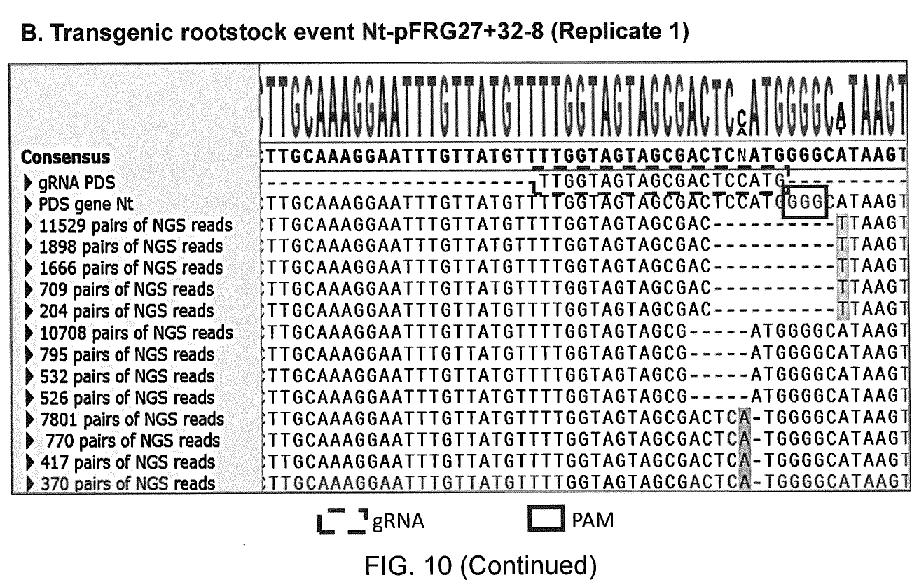

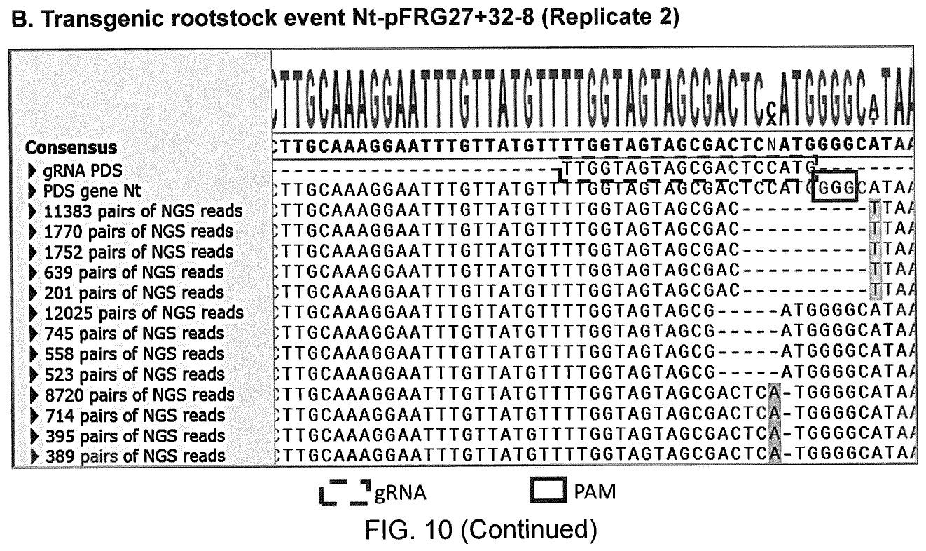

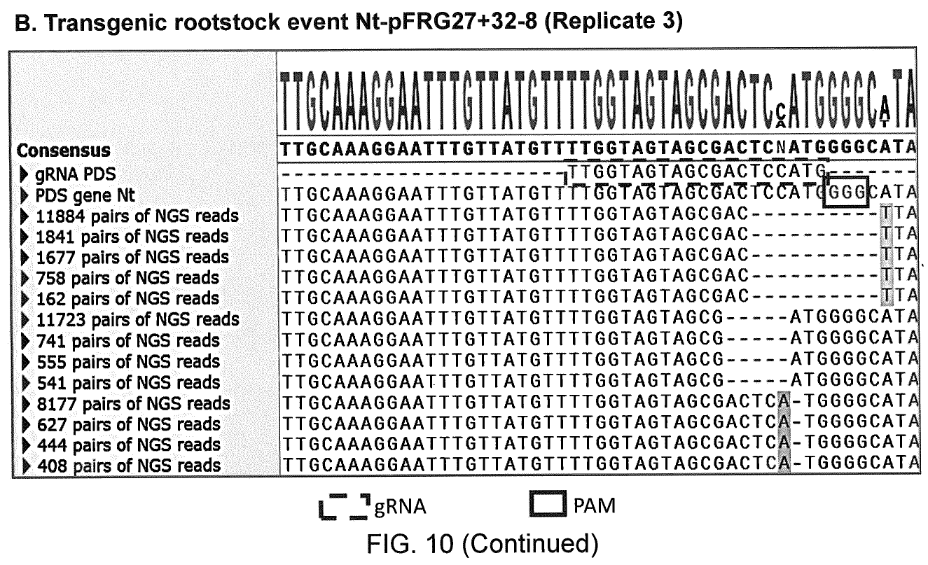

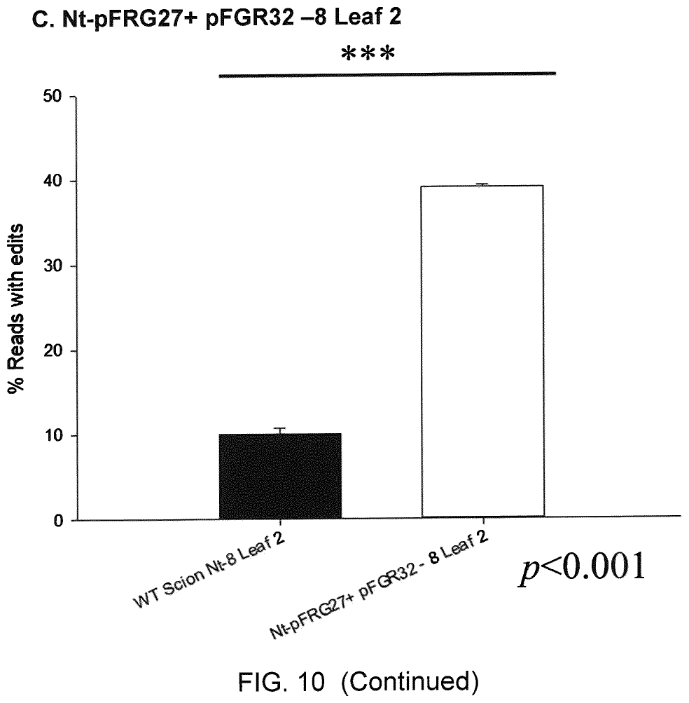

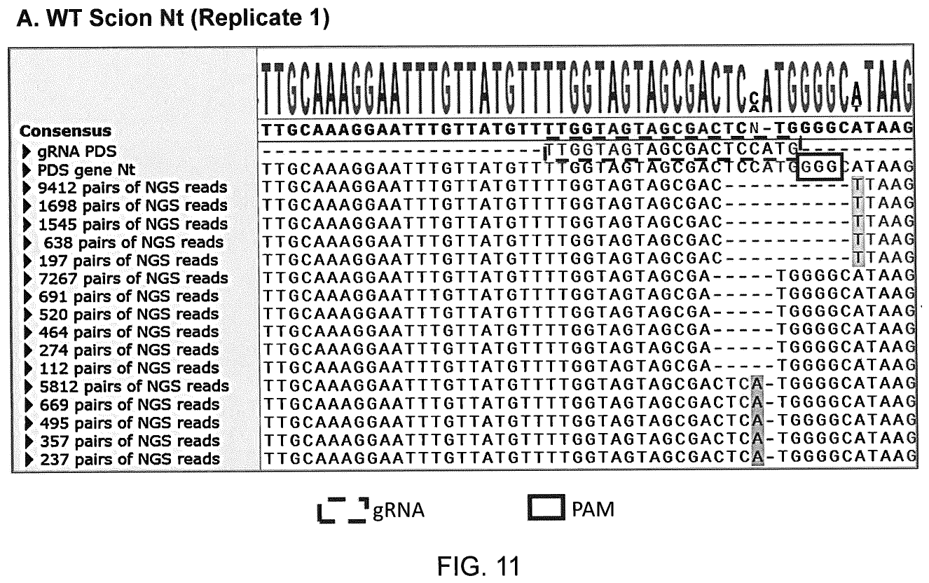

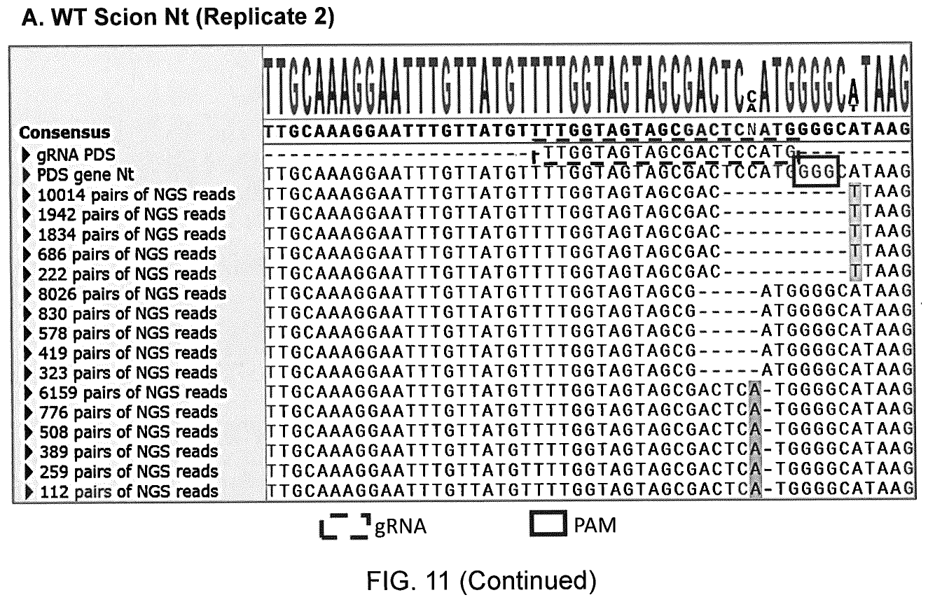

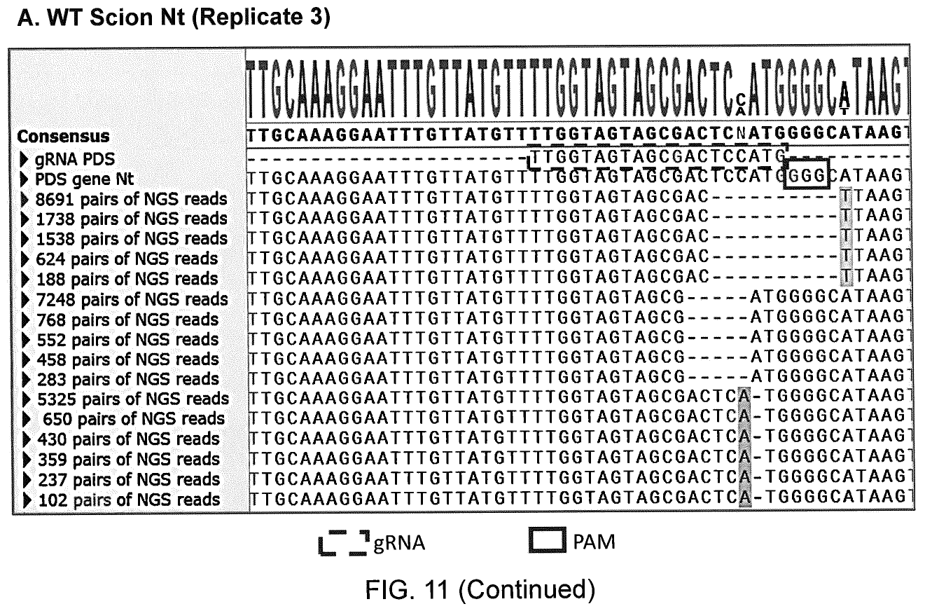

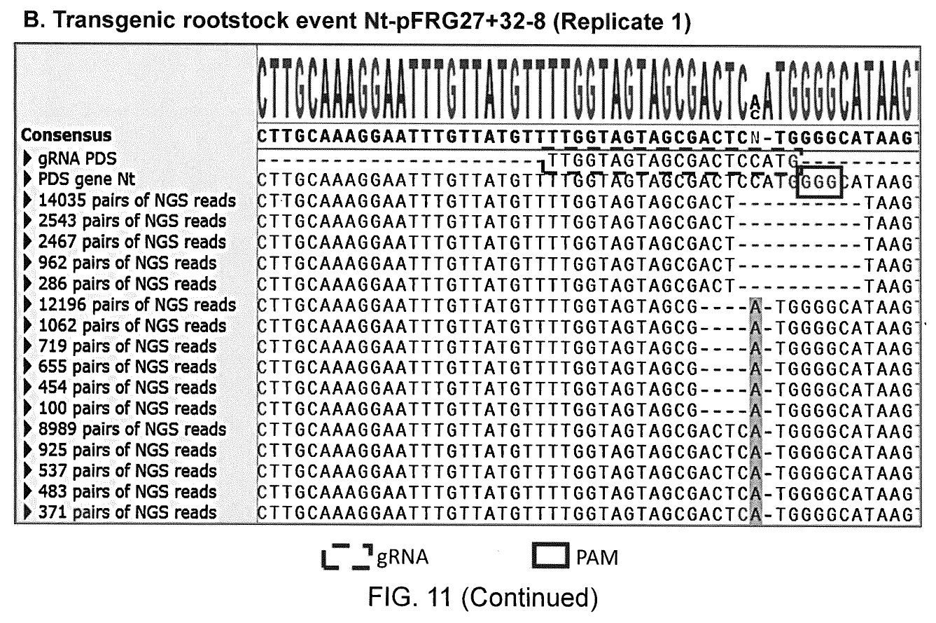

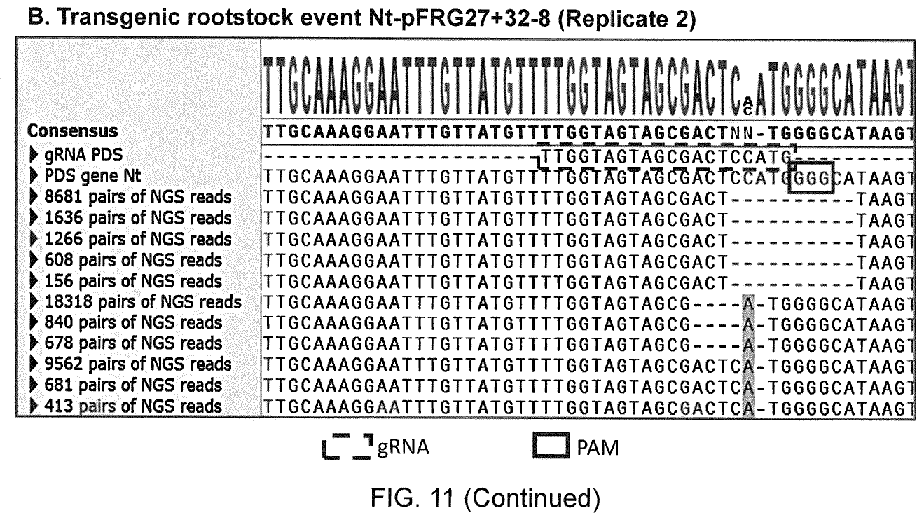

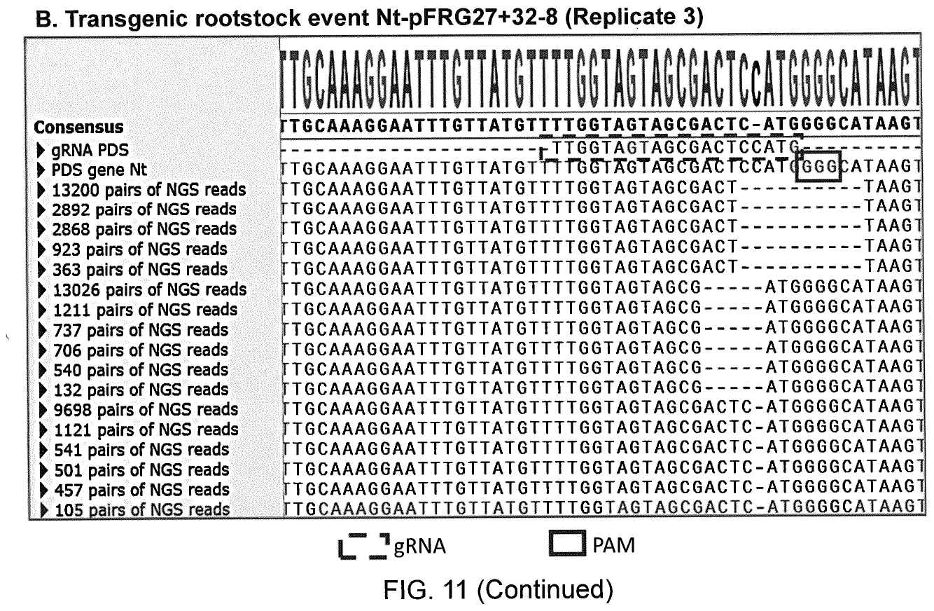

Molecular characterization of transgenic events was also performed. Since perturbations in the NtPDS gene generate an albino phenotype, potential transgenic plants were screened visually to identify albino plants (FIG. 2; Panels A-E) and subsequently evaluated using PCR to confirm the presence of NptII gene using FGRP5 and FGRP56 primers. PCR was performed using Dreamtaq (Thermofisher Scientific, EP0701) with an annealing temperature of 57° C. A total of 13 independent events were confirmed using PCR. Edits in the NtPDS gene were confirmed using Illumina high throughput sequencing of the targeted region amplicon at the CCIB DNA Core Facility at Massachusetts General Hospital Cambridge, MA (FIGS. 3-11).

The PCR products were first cleaned using QIAquick PCR purification kit (Qiagen®, 28104). An aliquot of cleaned 45 μL PCR product suspended in 10 mM Tris-HCl (pH8.0) was sequenced on an Illumina Next Generation Sequencing platform (CCIB DNA Core Facility at Massachusetts General Hospital. Cambridge, MA). The paired-end reads were processed in CLC Genomics Workbench (21.0.4). Reads were trimmed and filtered for a quality limit of 0.001, a maximum number of ambiguities of 2, a maximum length of 150, and a minimum length of 50. Illumina reads were then mapped to the NtPDS reference sequence (NCBI reference XM_016642616.1) with a length threshold of 40% and a similarity threshold of 70%. Mapped reads were extracted, and the CRISPR variants were verified by comparing the changes in the target region of the NtPDS gene. Indels observed within the gRNA sequence region were counted as edits in the NtPDS gene.

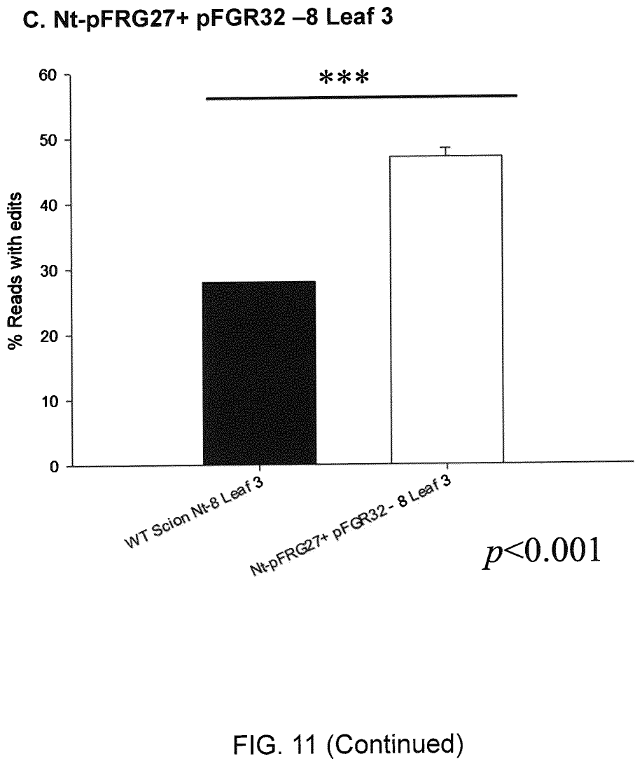

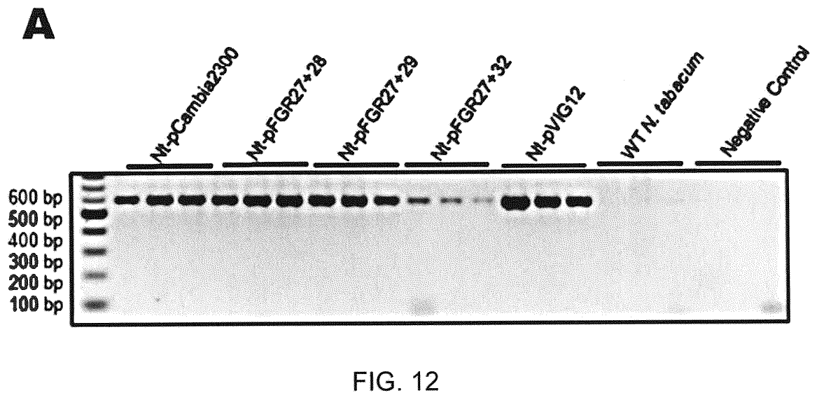

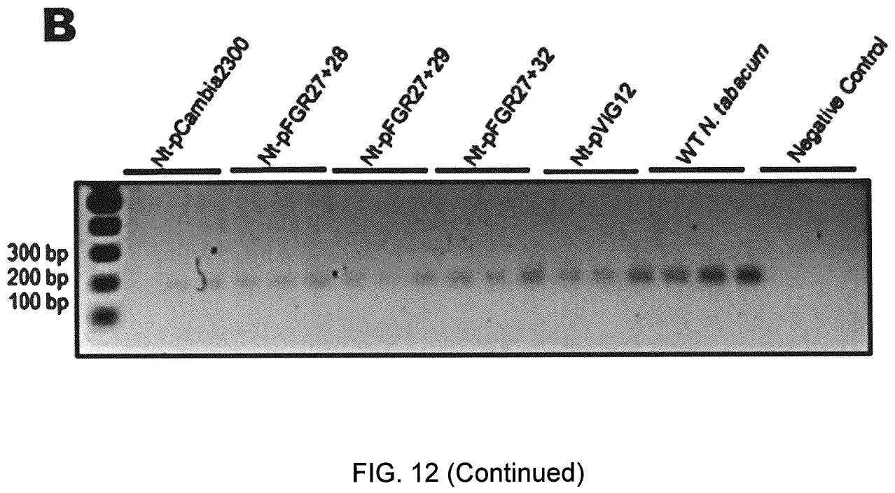

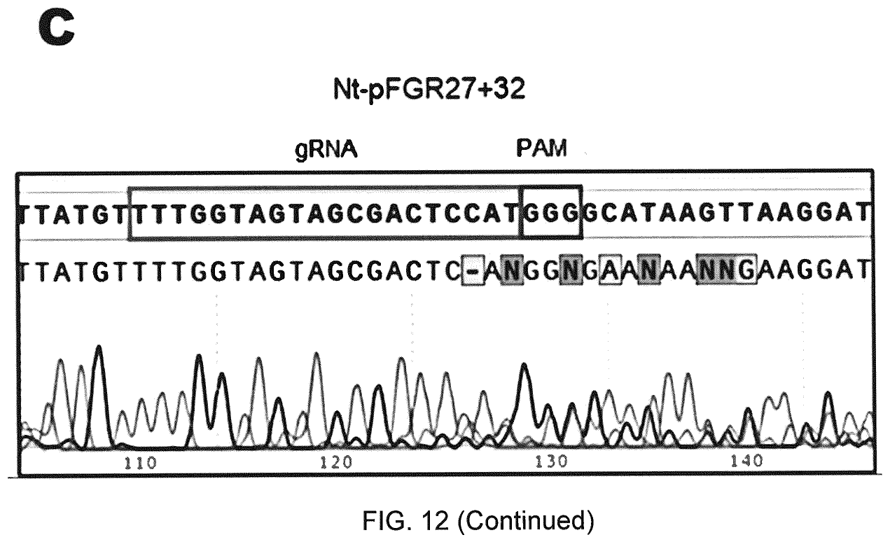

The exemplary data provided herein demonstrates that gene editing (e.g. NtPDS) via stable expression of TRV RNA1 and TRV RNA2-CRISPR/Cas9/gRNA targeting the NtPDS gene in Nicotiana tabacum (FIG. 2). Specifically, the efficiency of gene editing using pFGR32-T3 (TRV-CRISPR/Cas9, NtPDS gRNA) was similar to the efficiency obtained with the control pVIG12 plasmid (T6) (Table 3; 67.5% and 68%, respectively). The editing of the NtPDS gene was confirmed by amplifying and sequencing the target region of the NtPDS gene (FIG. 12).

TABLE 3
Summary of editing efficiency in transformed Nicotiana tabacum.

| | | No. of | Efficiency |
|---|---|---|---|
| | No. of | albino plants | of gene |
| Treatment | Explants | per event | editing |
| pCambia 2300 (FIG. 2; Panel A) | 18 | 0 | N/A |
| pFGR27 + 28 (FIG. 2; Panel B) | 15 | 0 | N/A |
| pFGR27 + 29 (FIG. 2; Panel C) | 15 | 0 | N/A |
| pFRG27 + 32 (FIG. 2; Panel D) | 40 | 27 | 67.5% |
| pVIG12 (FIG. 2; Panel E) | 22 | 15 | 68% |

### Example 3. Intra-Species Grafting for Delivery of Gene Editing Reagents

The transgenic N. tabacum lines with confirmed NtPDS edits were used as a rootstock in the following intra-species grafting experiments. Specifically, three independent transgenic events of Nt-pFGR27+32, stably expressing TRV1, TRV2-CRISPR/Cas9 and gRNA targeting NtPDS gene were selected to determine if a WT graft companion will be edited by the movement of TRV RNA 1 and TRV RNA2 along with the gene editing reagents across the graft junction. Since NtPDS-edited lines are albinos, any edits in the WT graft companion should manifest as an albino phenotype in the non-transgenic tissue.

Three independent transgenic events with confirmed edits in the NtPDS gene were used as rootstock for grafting to determine the ability to deliver the CRISPR/Cas9/gRNA reagents to the non-transgenic scion. The rootstock plant was first decapitated, and a “v” shape cut was created. A non-transgenic scion of N. tabacum was prepared with an inverted “v” shape complementary to the rootstock. Once the rootstock and scion junctions were joined, a plastic clip was used to secure the graft junction.

Four weeks post-grafting, three leaf samples were collected from both the rootstock and the scion from each of the three independent grafted plants. Total cellular DNA was isolated using a DNeasy Plant kit (Qiagen® Cat. No./ID: 69204), and PCR was performed on three technical replicates representing each leaf with primers targeting TRV1 (RGP110 and FGRP159), TRV2-CRISPR/Cas9 (RGP111 and RGP112; FGRP8 and FGRP9), and gRNA (RCWP374 and RWCP375). The gRNA-targeted region within the NtPDS gene was also PCR-amplified and cleaned using agarose gel extraction with QIAquick gel extraction kit (Qiagen Inc.) before being sequenced using Illumina. All the primers and their sequences are listed in Table 4.

TABLE 4
Oligonucleotide primers used for Illumina sequencing, including their
respective sequences, target region, and expected amplicon sizes.

| | | | Expected | |
|---|---|---|---|---|
| Sequence | Name of | | Amplicon | |
| Identifier | primer | Description | size | |
| SEQ ID NO: 7 | FGRP277 | Forward primer for NtPDS gene | 180 | bp |
| | | Nicotiana tabacum | | |
| SEQ ID NO: 8 | FGRP278 | Reverse primer for NtPDS gene | | |
| | | Nicotiana tabacum | | |
| SEQ ID NO: 9 | FGRP257 | Forward primer for 5′ Cas9 | 1.2 | kb |
| SEQ ID NO: 10 | FGRP258 | Reverse primer for 5′ Cas9 | | |
| SEQ ID NO: 11 | FGRP253 | Forward primer for 3′ Cas9 | 1.6 | kb |
| SEQ ID NO: 12 | FGRP254 | Reverse primer for 3′ Cas9 | | |
| SEQ ID NO: 13 | FGRP259 | Forward for 5′ TRV RNA2 genes | 1.6 | kb |
| SEQ ID NO: 14 | FGRP260 | Reverse primer for 5′ TRV RNA2 genes | | |
| SEQ ID NO: 15 | FGRP261 | Forward primer for 3′ TRV RNA2 genes | 400 | bp |
| SEQ ID NO: 16 | FGRP262 | Reverse primer for 3′ TRV RNA2 genes | | |
| SEQ ID NO: 17 | RCWP374 | Forward primer for gRNA promoter and | 400 | bp |
| | | scaffold | | |
| SEQ ID NO: 18 | RCWP375 | Reverse primer for gRNA promoter and | | |
| | | scaffold | | |
| SEQ ID NO: 19 | FGRP263 | Forward primer for 5′ TRV RNA1 | 1.6 | kb |
| | | genome | | |
| SEQ ID NO: 20 | FGRP264 | Reverse primer for 5′ TRV RNA1 | | |
| | | genome | | |
| SEQ ID NO: 21 | FGRP273 | Forward primer for 3′ TRV RNA1 | 700 | bp |
| | | genome | | |
| SEQ ID NO: 22 | FGRP274 | Reverse primer for 3′ TRV RNA1 | | |
| | | genome | | |
| SEQ ID NO: 23 | FGRP5 | Forward primer for NPTII gene | 600 | bp |
| SEQ ID NO: 24 | FGRP56 | Reverse primer for NPTII gene | | |
| SEQ ID NO: 25 | BWBP268 | Forward NtPDS gRNA N. tabacum | N/A | |
| SEQ ID NO: 26 | BWBP269 | Reverse NtPDS gRNA N. tabacum | N/A | |
| SEQ ID NO: 27 | FGRP3 | Forward primer to amplify NptII gene | 1.76 | kb |
| | | from pCambia2300 | | |
| SEQ ID NO: 28 | FGRP4 | Reverse primer to amplify NptII gene | | |
| | | from pCambia2300, SbfI site added | | |
| SEQ ID NO: 29 | FGRP165 | Forward primer to amplify TRV RNA2 | 3.3 | kb |
| | | genome | | |
| SEQ ID NO: 30 | FGRP166 | Reverse primer to amplify TRV RNA2 | | |
| | | genome PmeI site added | | |
| SEQ ID NO: 31 | FGRP8 | Forward primer for Cas9 in Nicotiana | 4.2 | kb |
| | | tabacum plants | | |
| SEQ ID NO: 32 | FGRP9 | Reverse primer for 5′ Cas9 in Nicotiana | | |
| | | tabacum plants | | |
| SEQ ID NO: 33 | RGP110 | Forward primer for TRV1 RNA1 | 3 | kb |
| | | genome in Nicotiana tabacum plants | | |
| SEQ ID NO: 34 | FGR159 | Reverse primer for TRV RNA1 genome | | |
| | | in Nicotiana tabacum plants | | |
| SEQ ID NO: 35 | RGP111 | Forward primer for TRV RNA2 genome | 350 | bp |

| | | |
|---|---|---|
| | | in Nicotiana tabacum plants |
| SEQ ID NO: 36 | RGP112 | Reverse primer for TRV RNA2 genome |
| | | in Nicotiana tabacum plants |

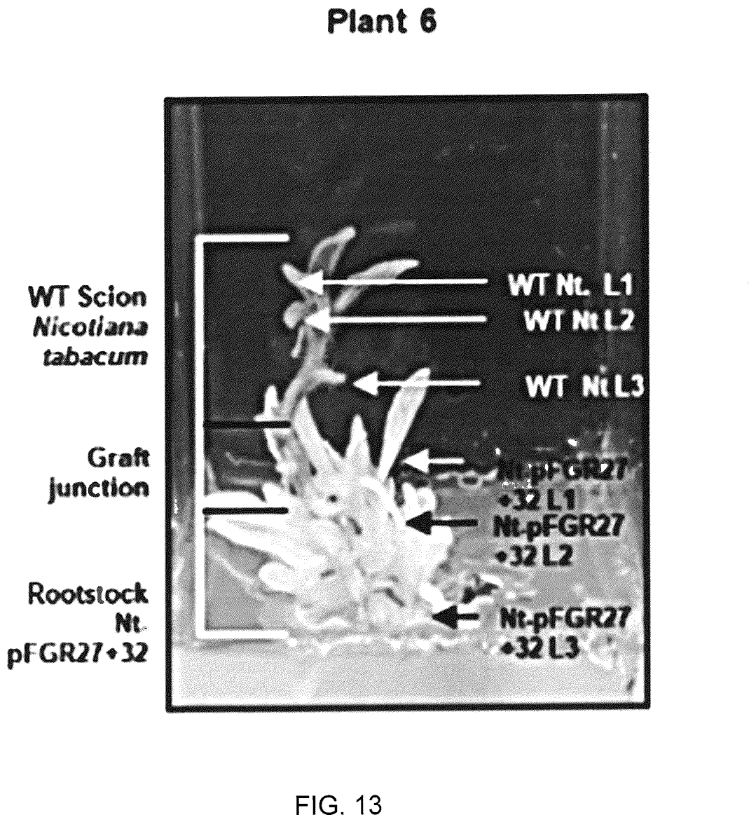

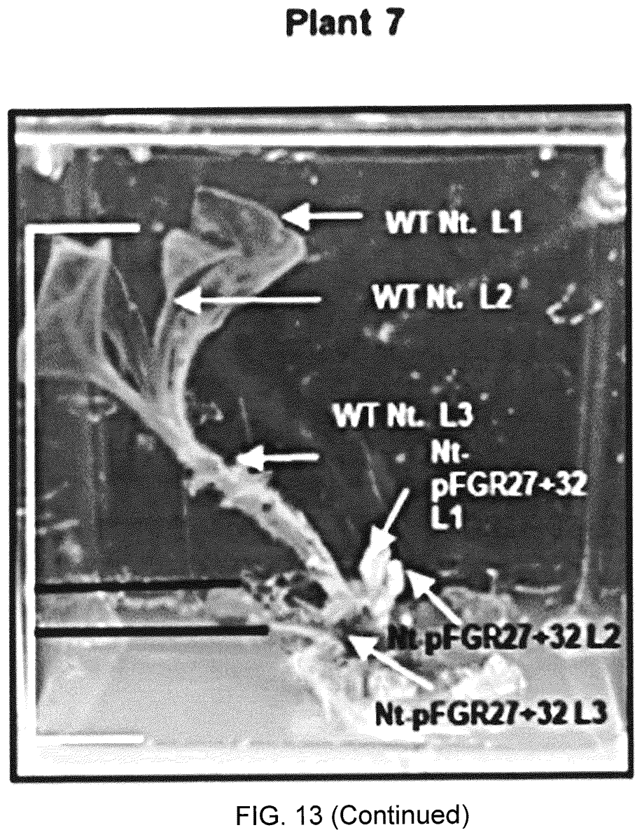

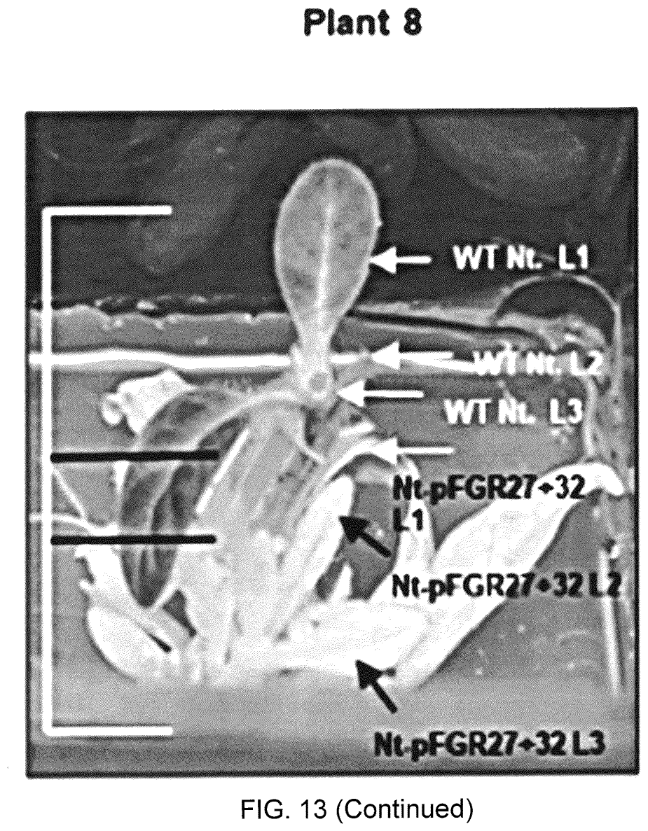

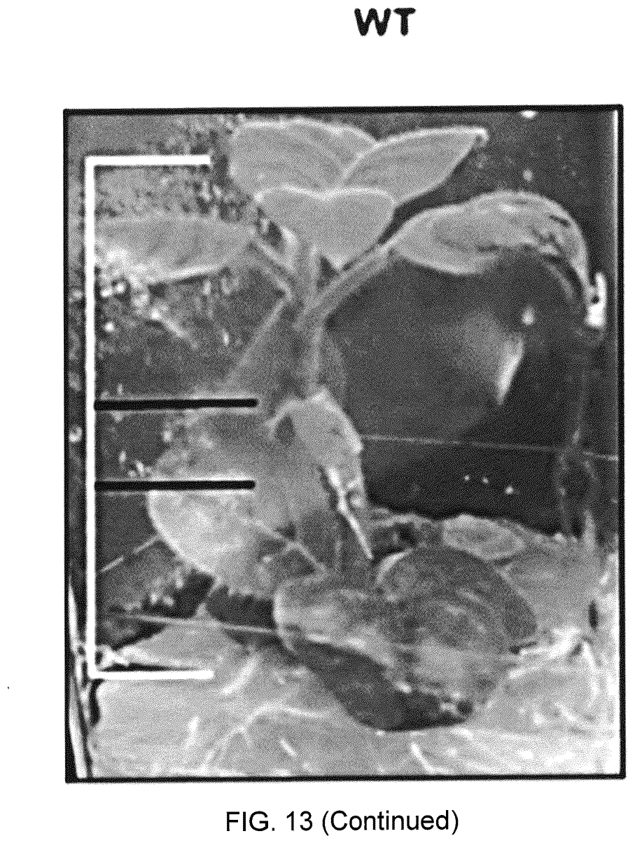

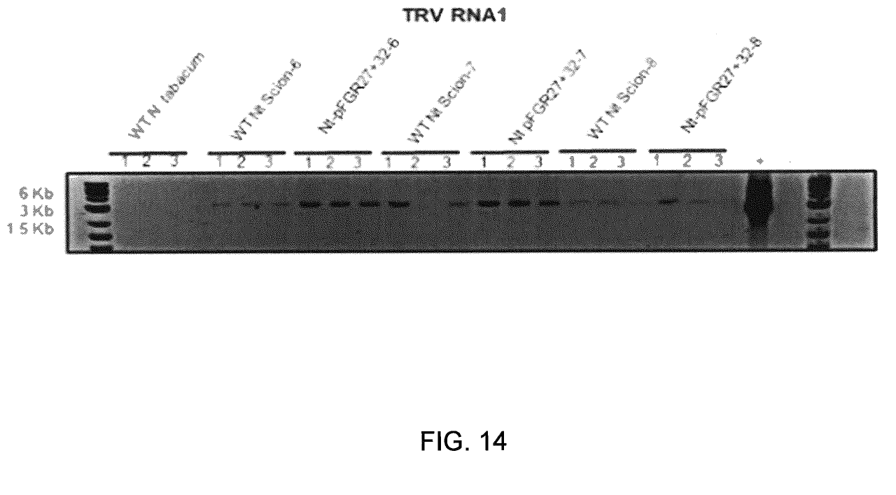

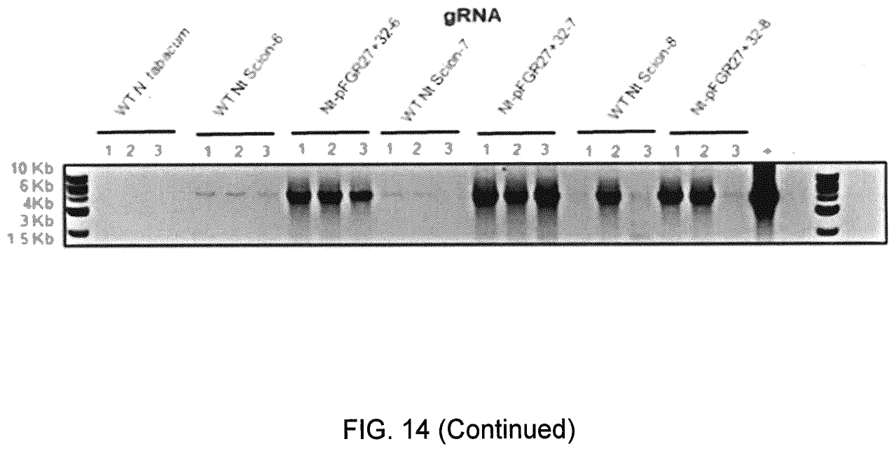

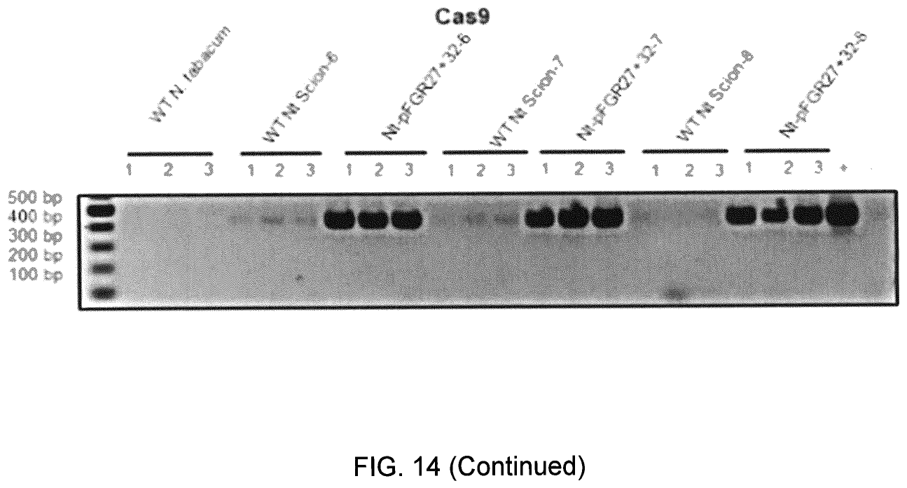

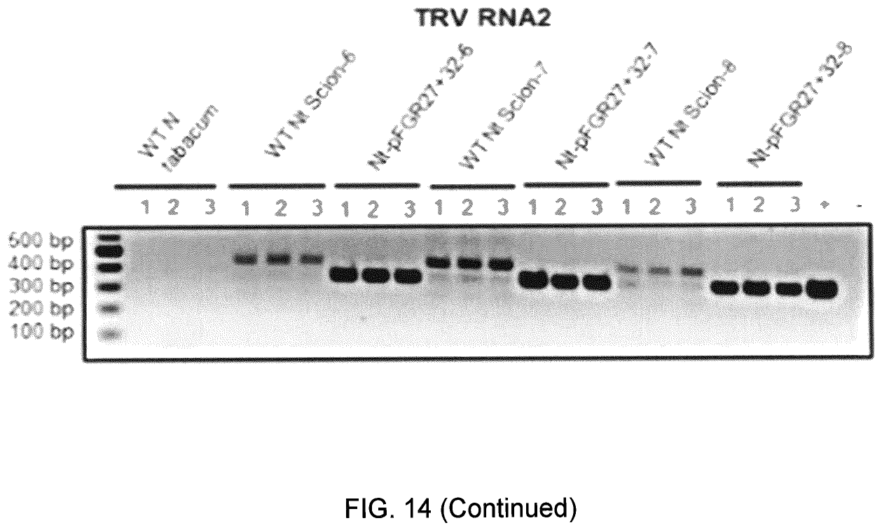

Since NtPDS-edited lines generally exhibit albino/pale green phenotype, any edits within the WT graft companion should manifest similar phenotype. After 4-6 weeks of grafting, pale green phenotype was observed in the WT scions (FIG. 13) indicating that the target gene may be edited due to the delivery of the CRISPR/Cas9 reagents across the graft junction.

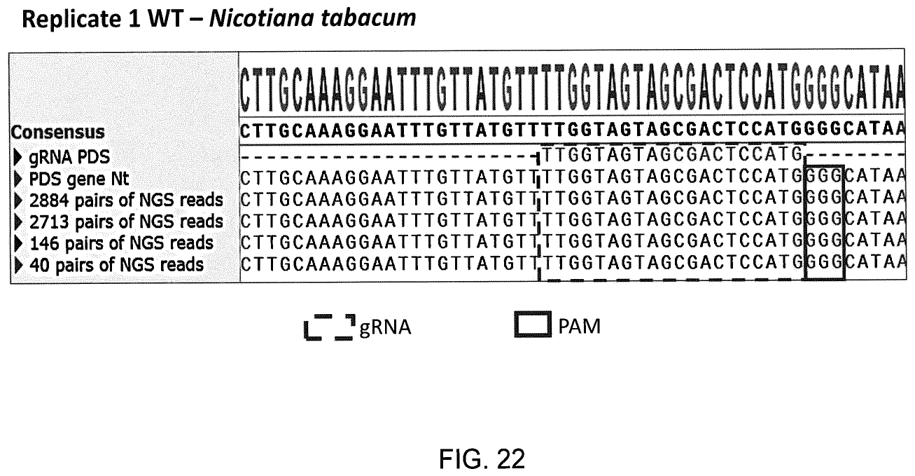

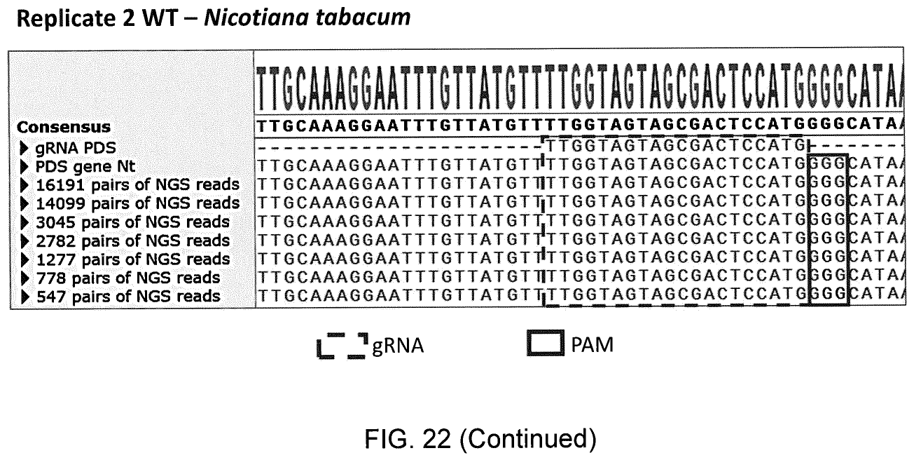

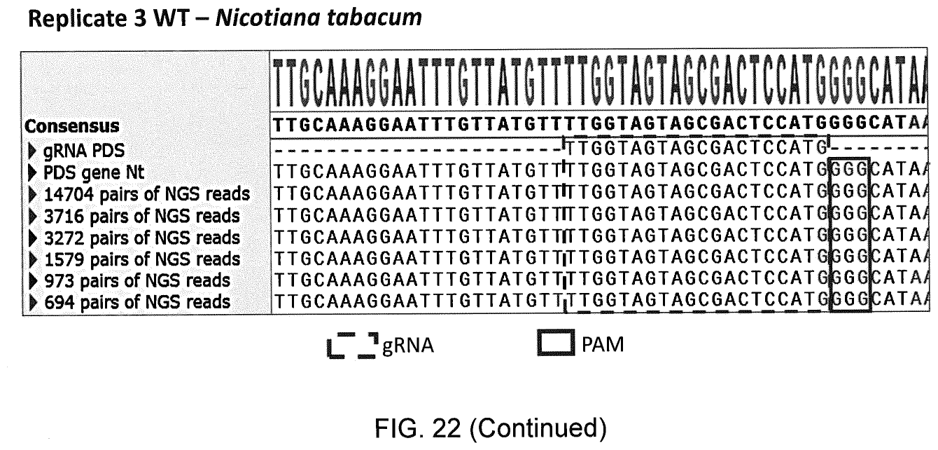

To confirm that all the gene editing components were indeed being transferred to the WT scion tissue, PCR was performed to detect individual CRISPR components in three leaves each derived from three separately grafted wild type scions as well as the transgenic donor lines (FIG. 13). Subsequently, whether TRV1/TRV2-mediated delivery of the CRISPR/Cas9 reagents will edit the targeted gene was evaluated. Amplicon sequencing of the targeted region within the NtPDS gene was performed using three leaves each (with three technical replicates) from the WT scion tissue as well as the transgenic rootstock tissue. The sequencing reads in WT samples showed no edits as expected (FIG. 22).

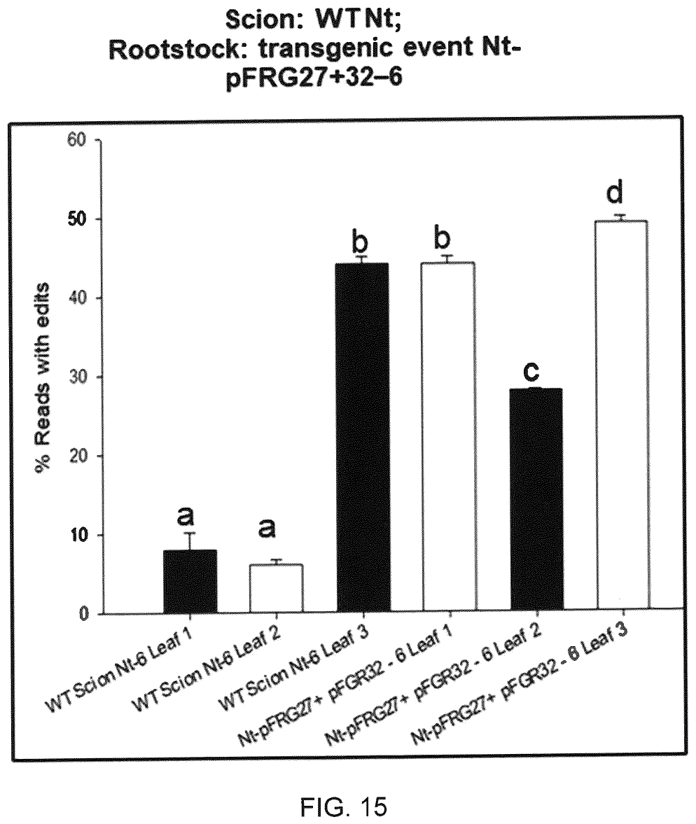

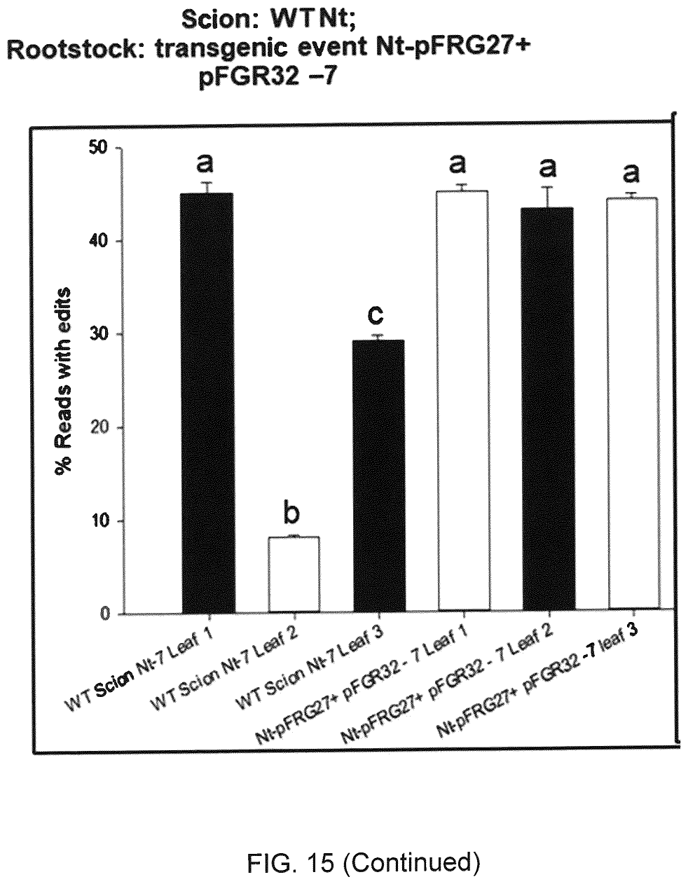

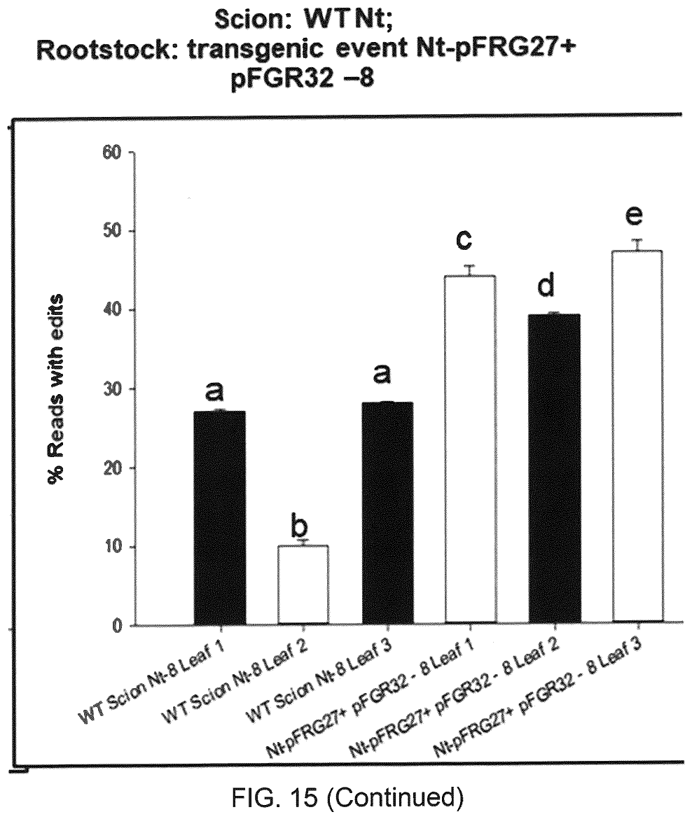

Successful gene editing was evident in the NtPDS gene in the WT scion tissue. The percentage of edited reads detected in the transgenic donor tissue was nearly 50%, while in the scion tissue the percentage of edited reads ranged from 10-50% (FIG. 15). Intriguingly, only 50% of the reads were edited in the transgenic donor rootstocks. In the case where Nt-pFGR27+32-6, leaf 3 from the WT scion demonstrated up to 44% of edited reads (FIG. 15). Similarly, WT scion leaf 1 in the case where Nt-pFGR27+32-7 was used as the rootstock demonstrated the highest percentage of edited reads up to 45% (FIG. 15). However, in case of the third transgenic event Nt-pFGR27+32-8, the percentage of edited reads in the three leaves derived from the WT scion demonstrated a lower number of edits from 10% to 28% (FIG. 15).

### Example 4. Transmission of Gene Editing Components and Maintenance of the Edits Following Organogenesis

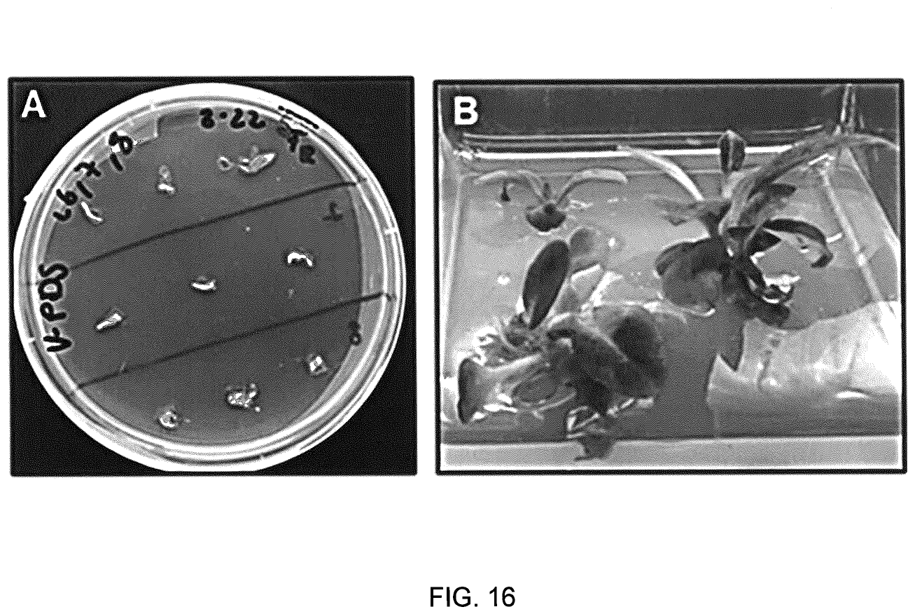

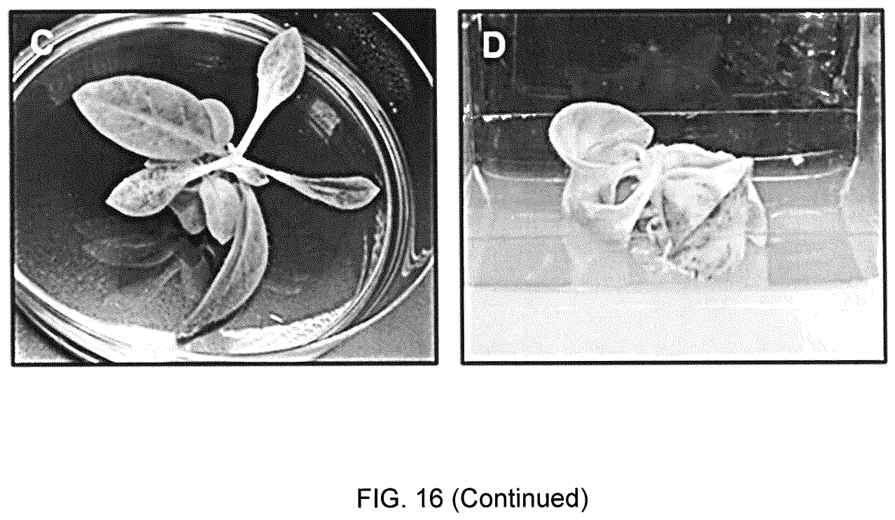

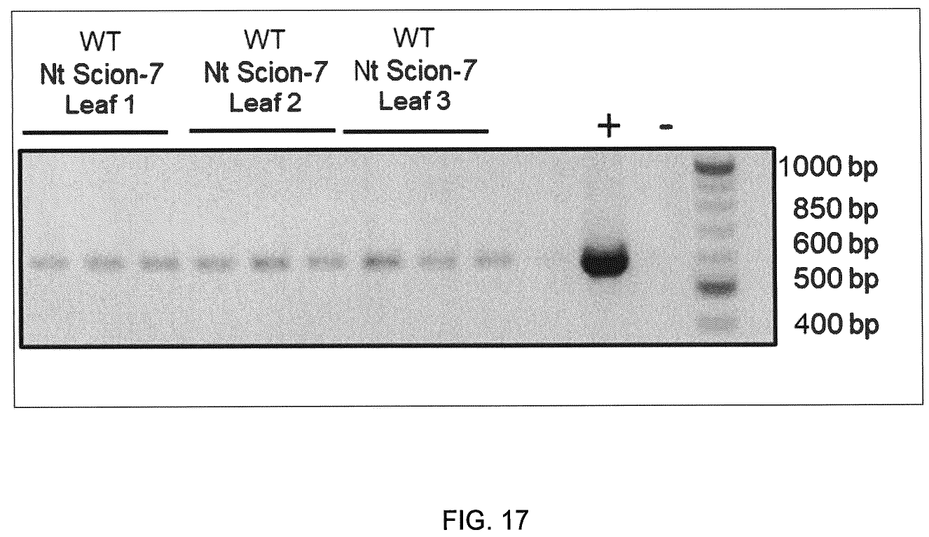

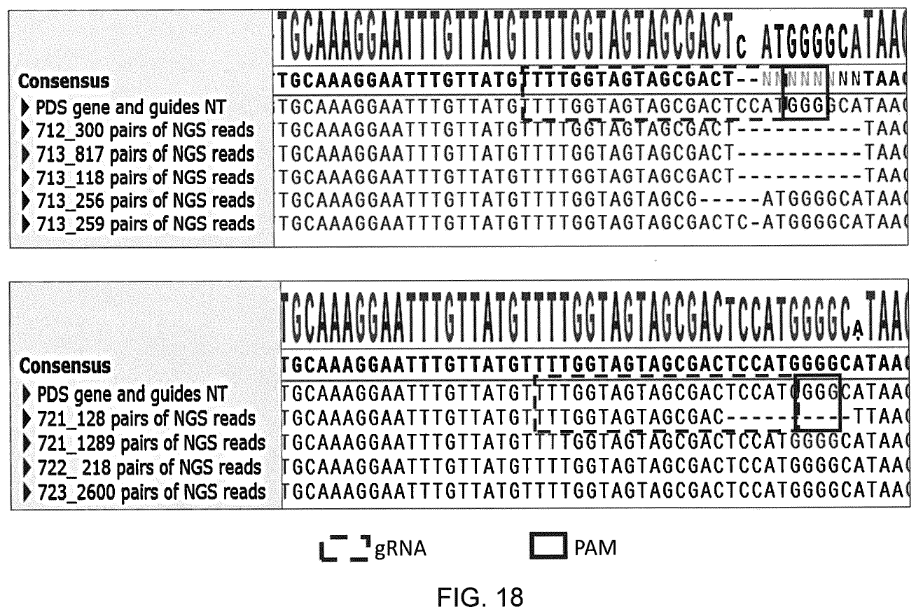

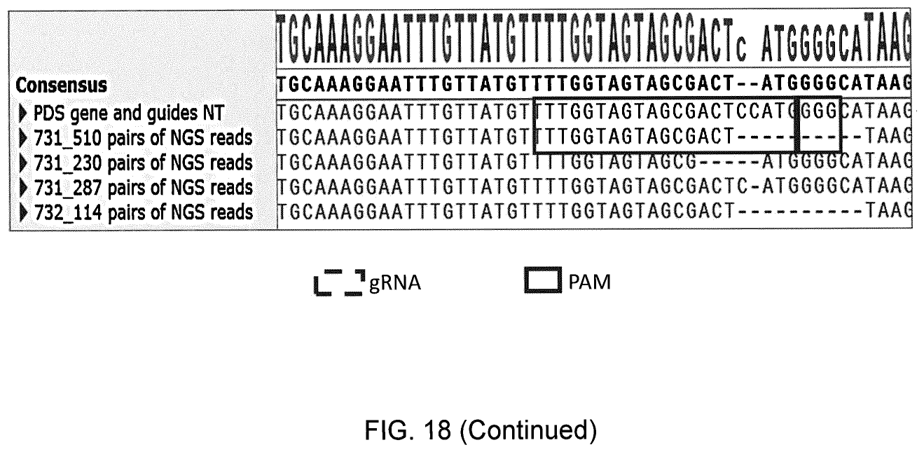

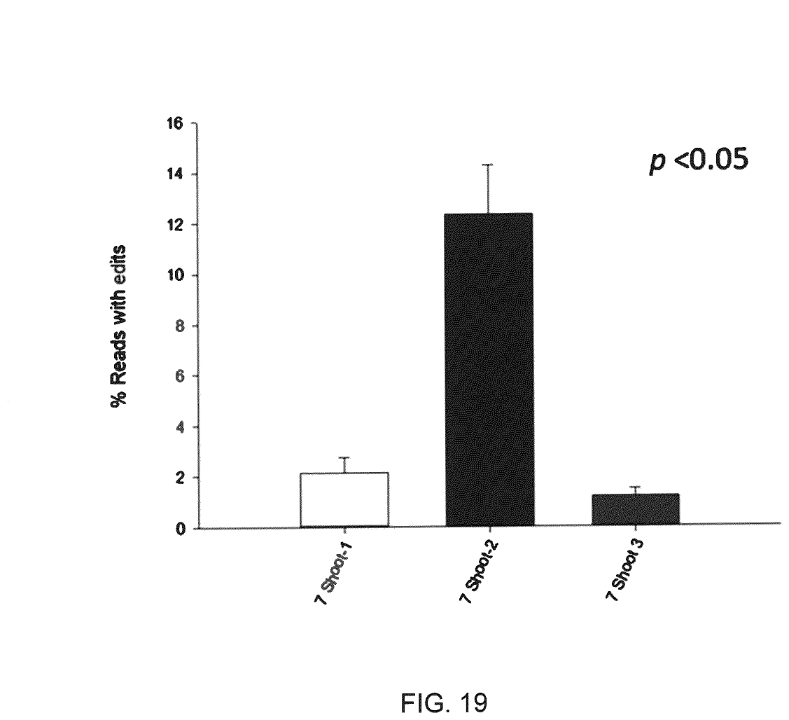

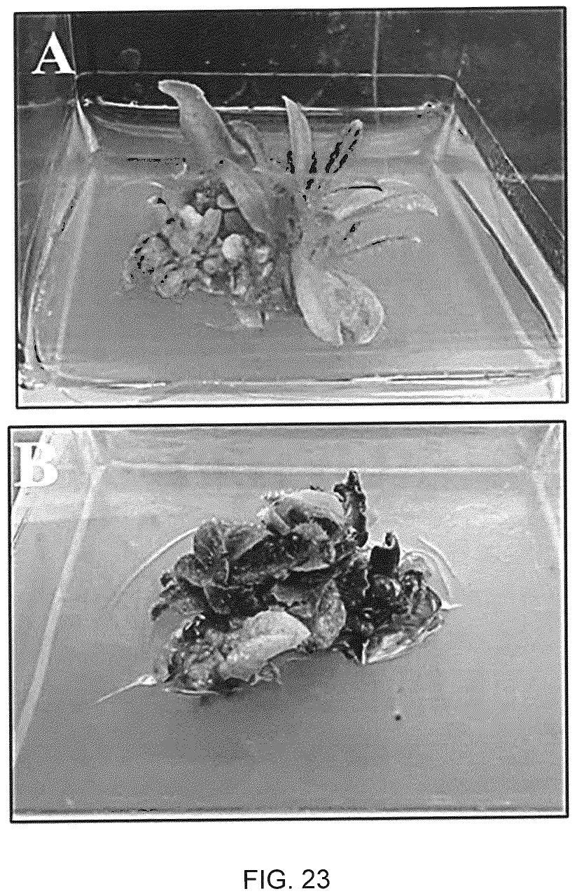

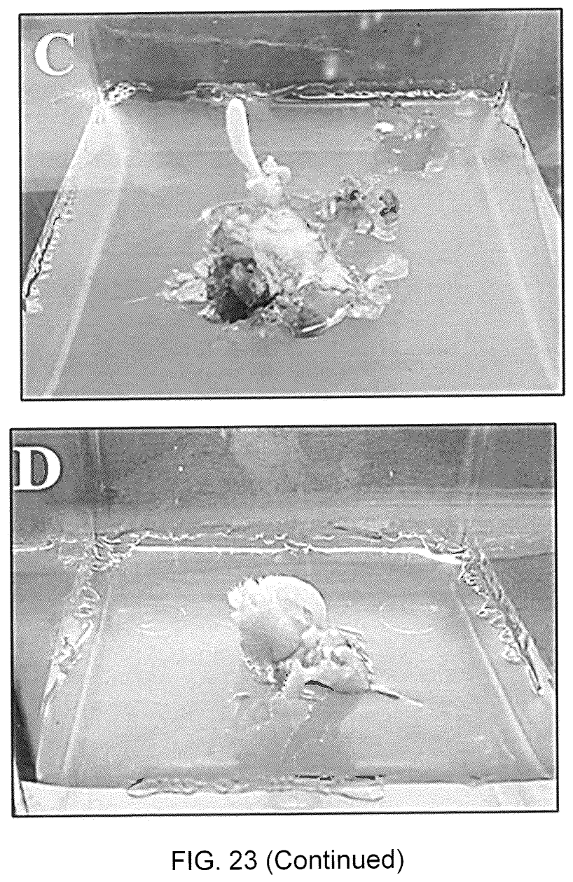

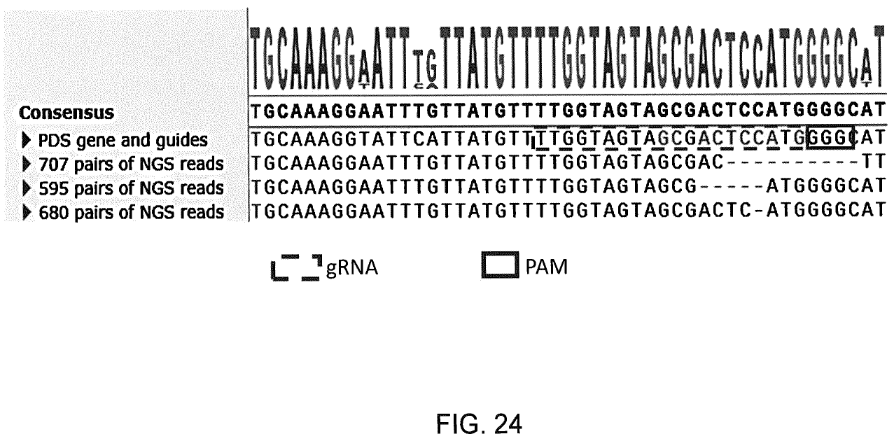

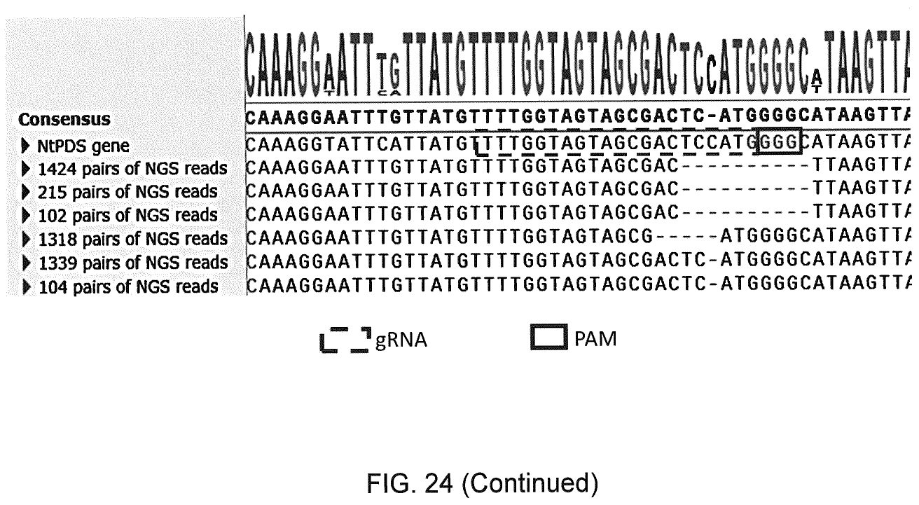

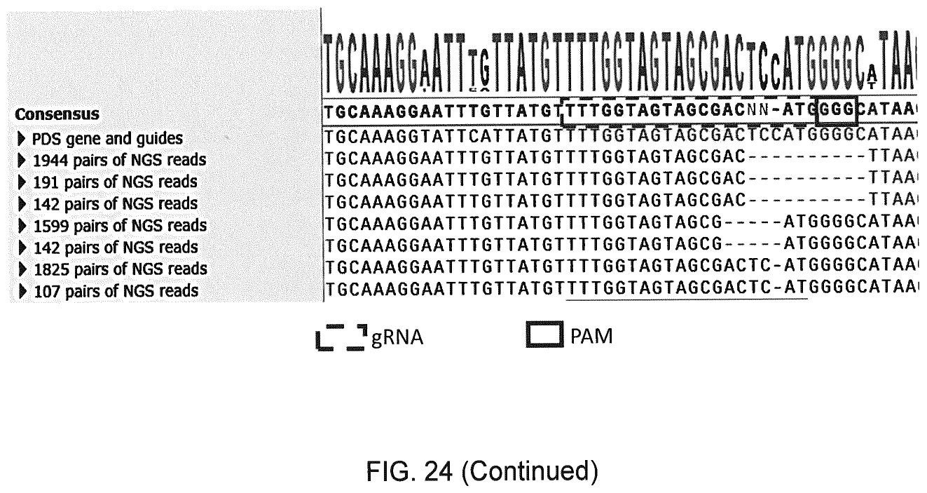

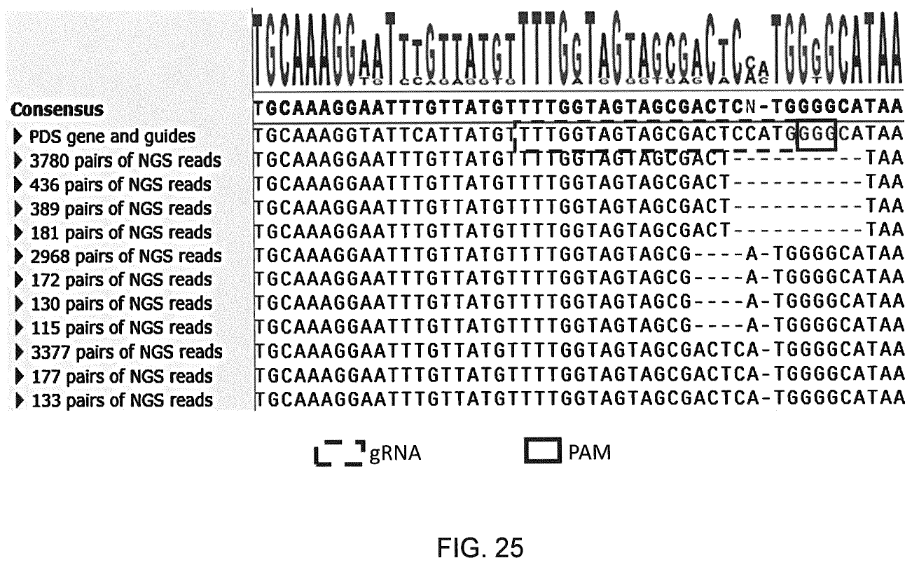

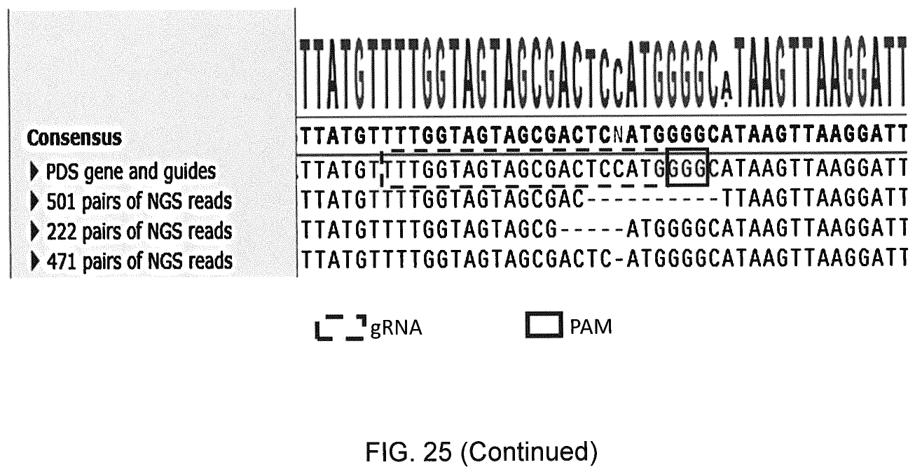

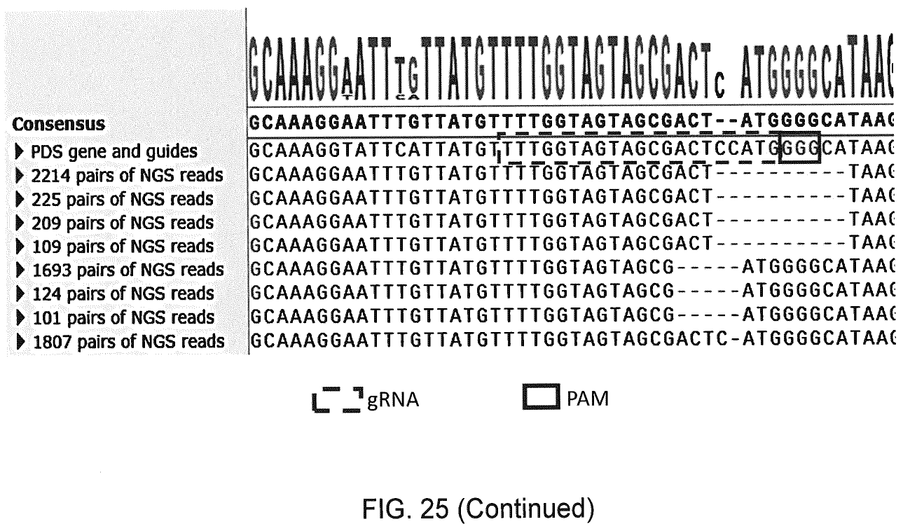

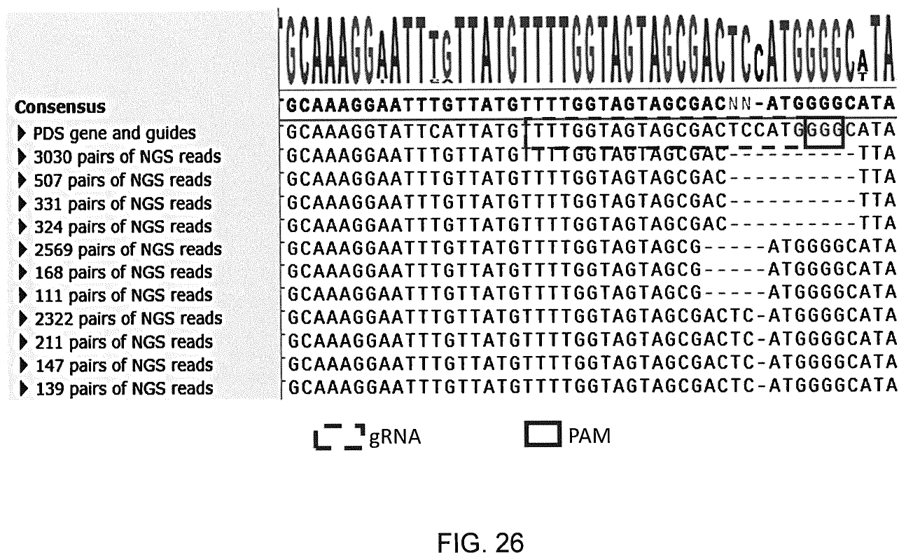

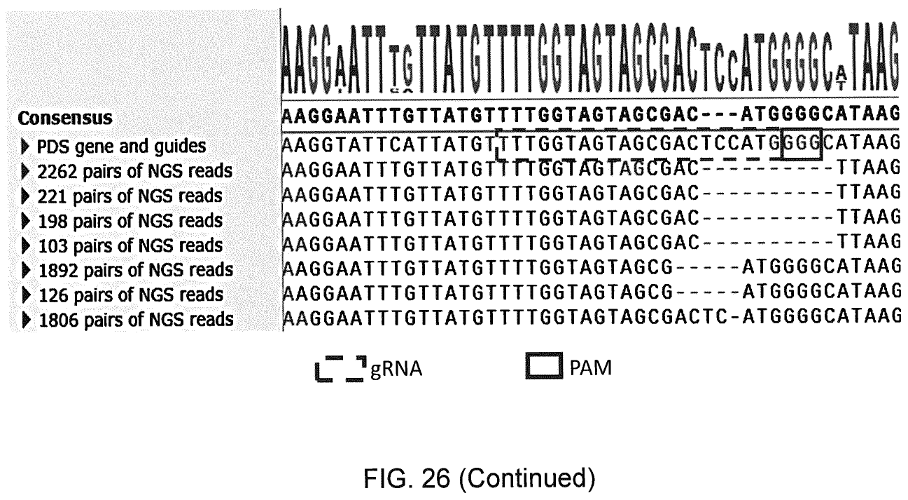

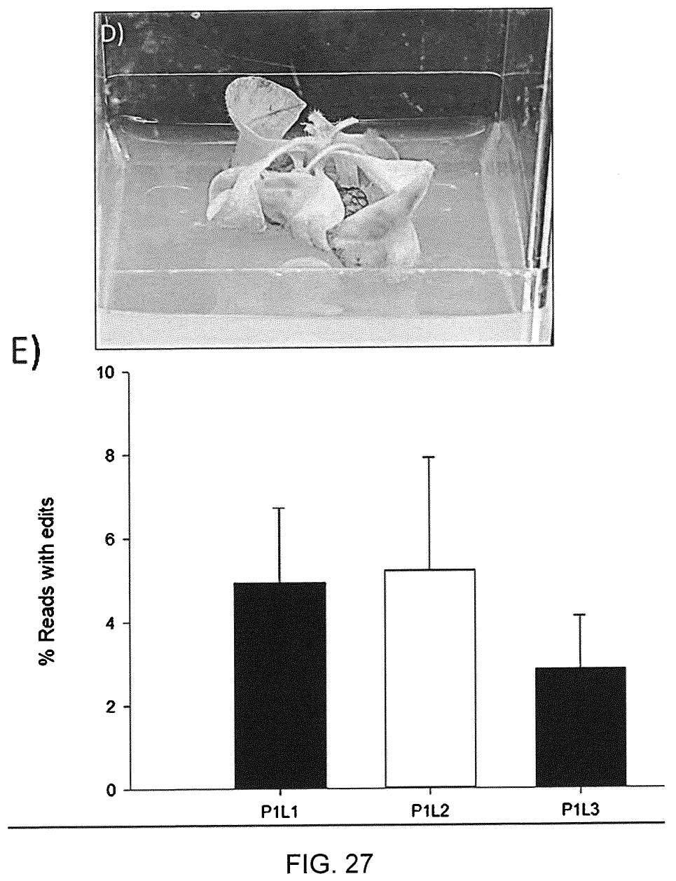

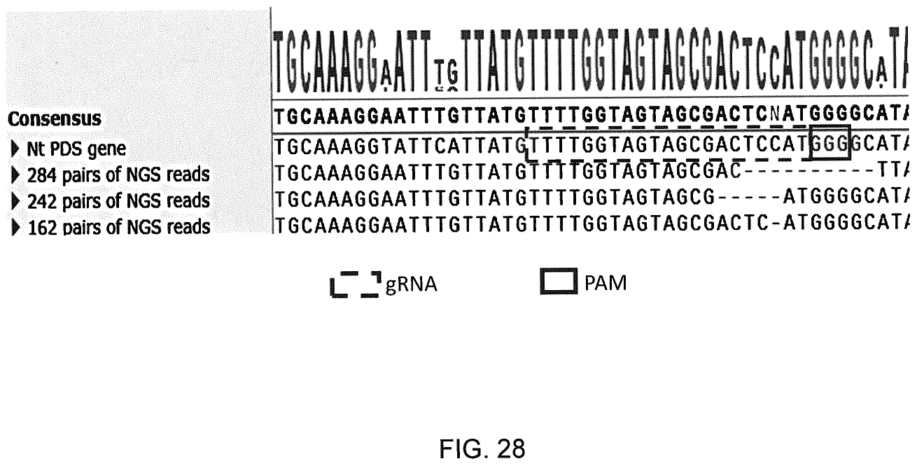

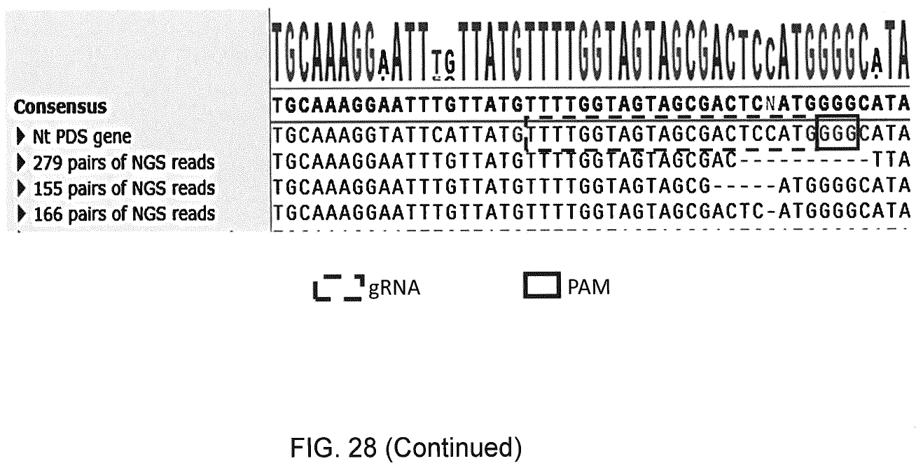

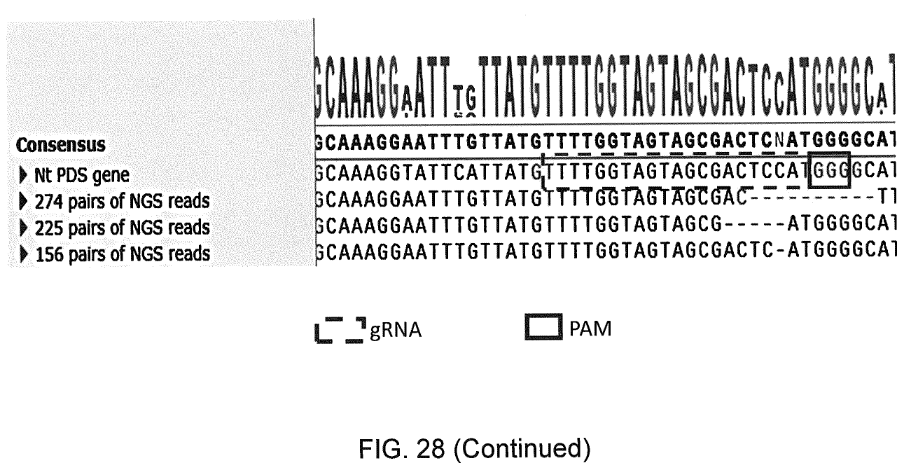

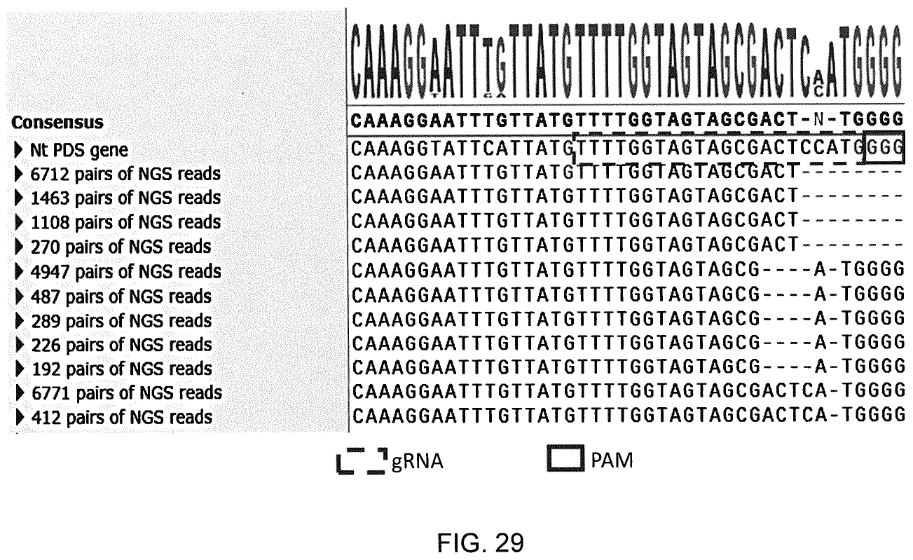

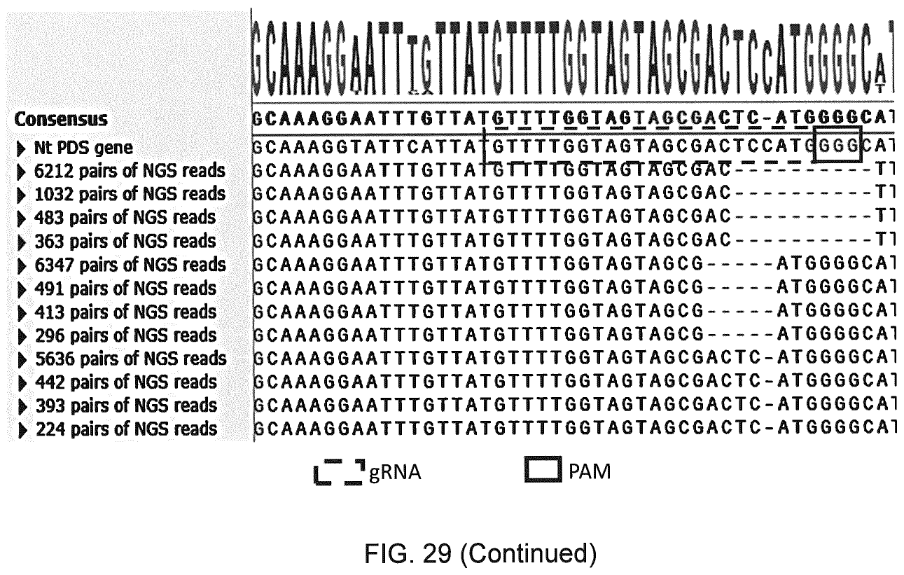

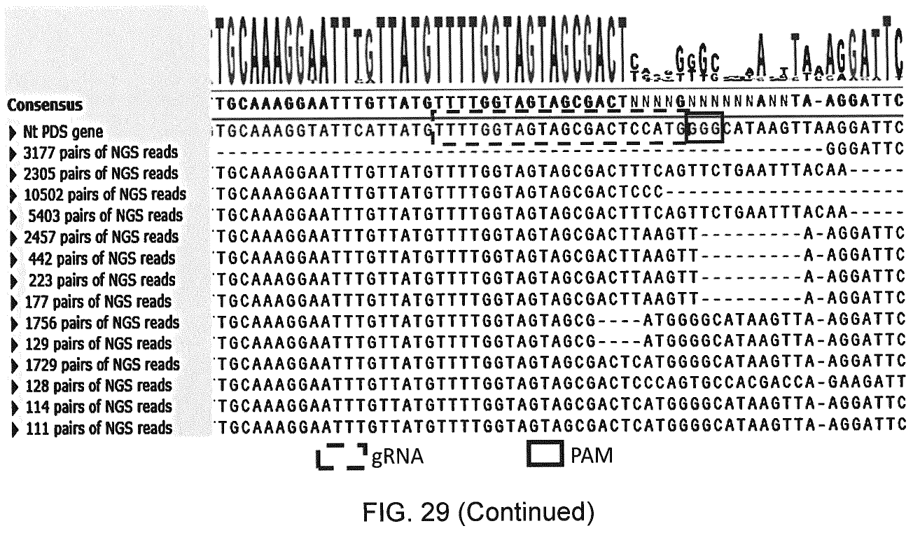

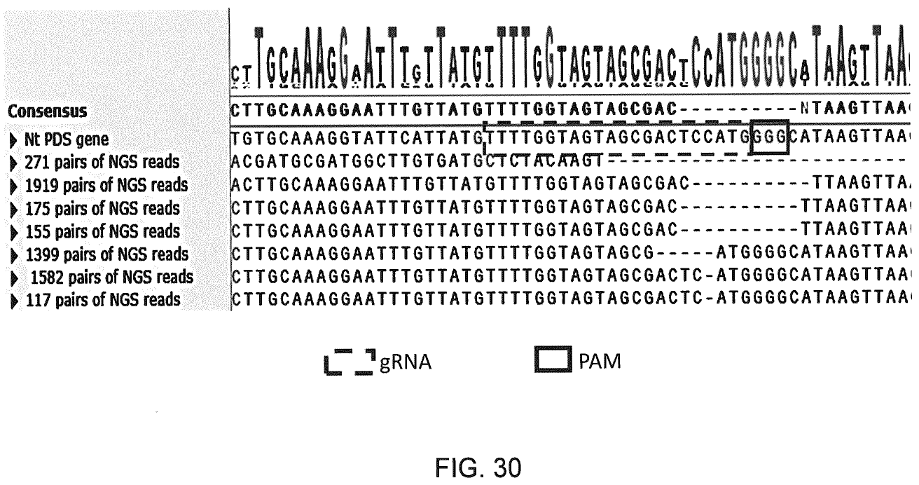

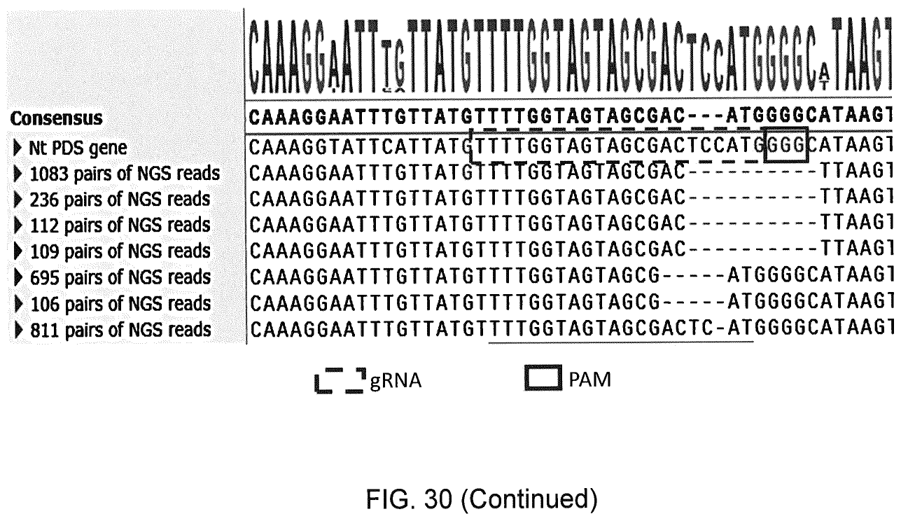

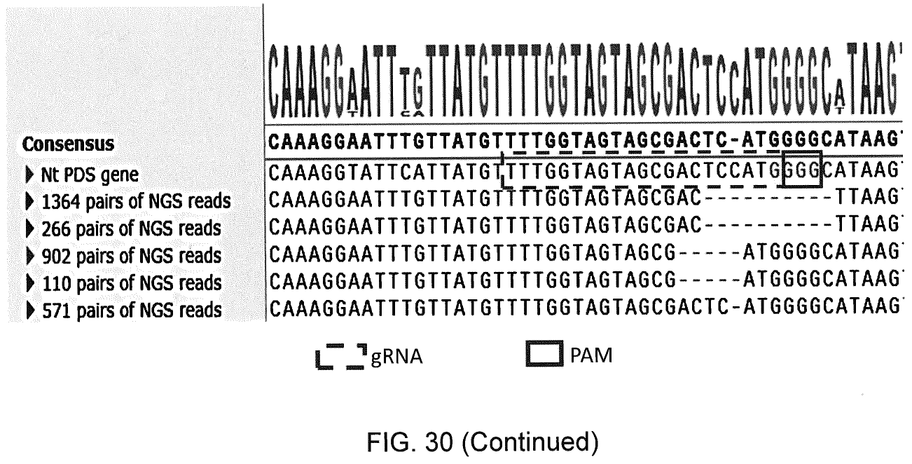

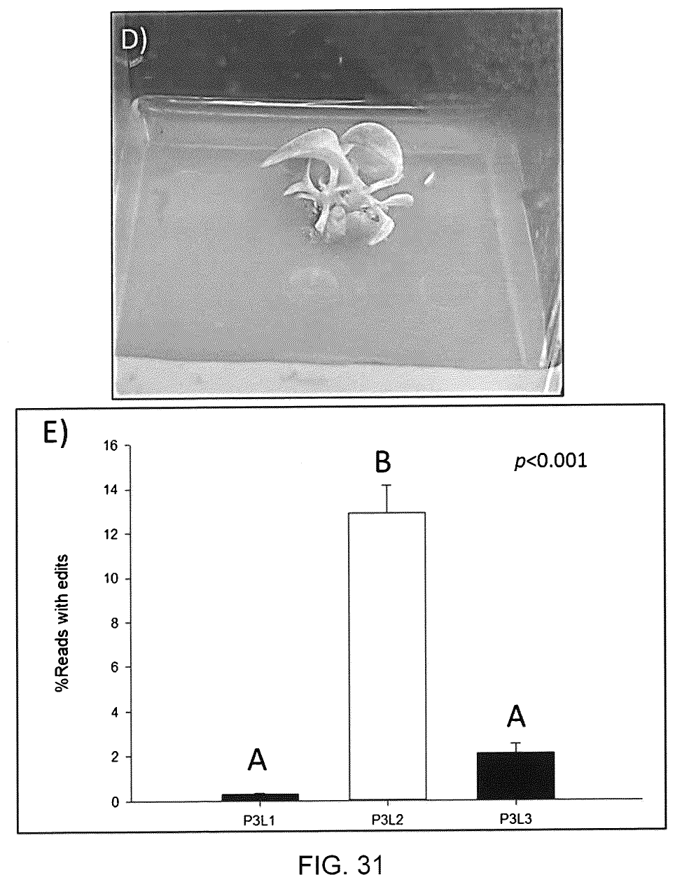

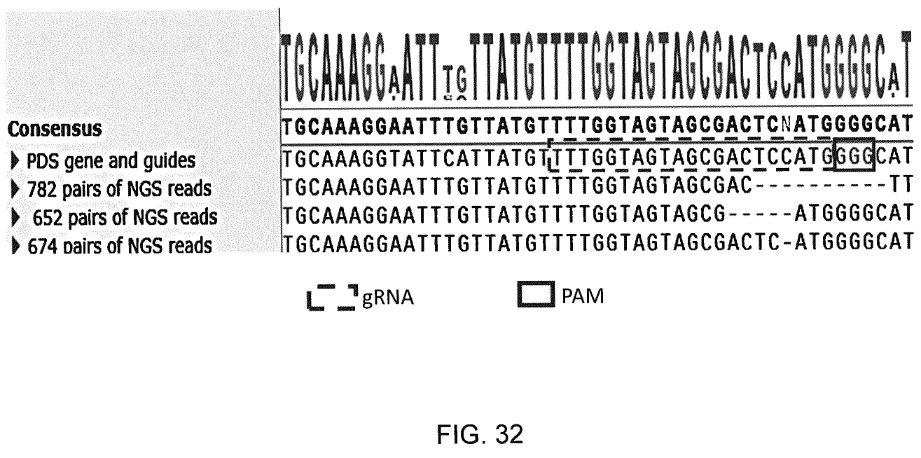

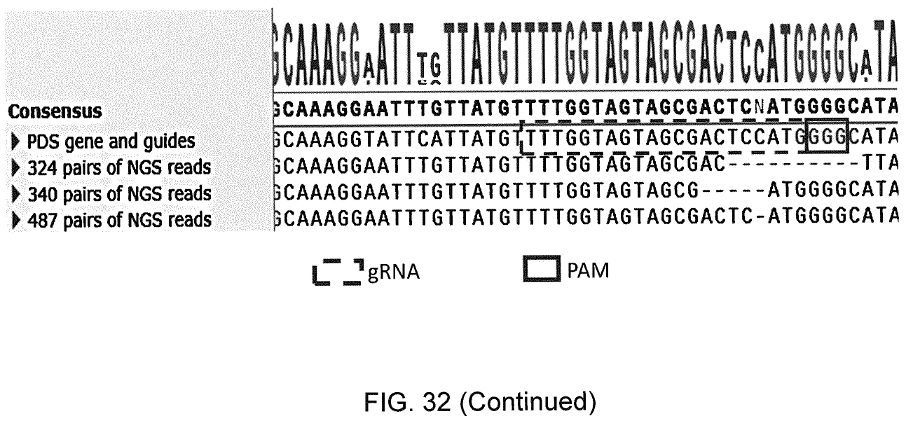

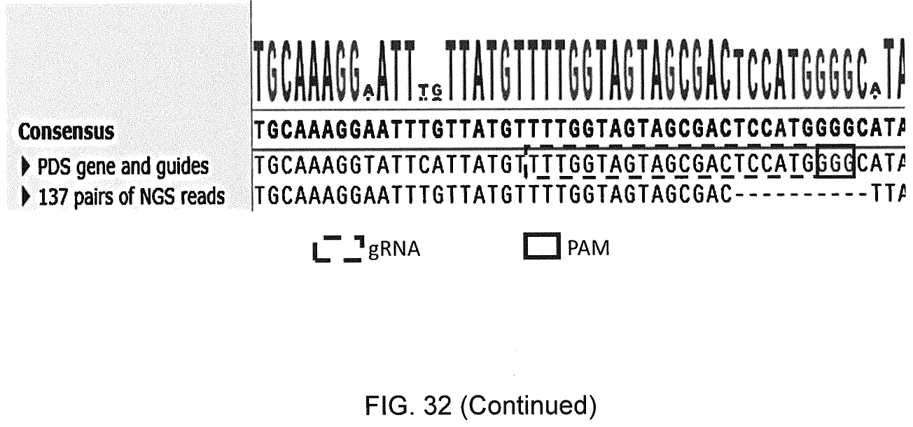

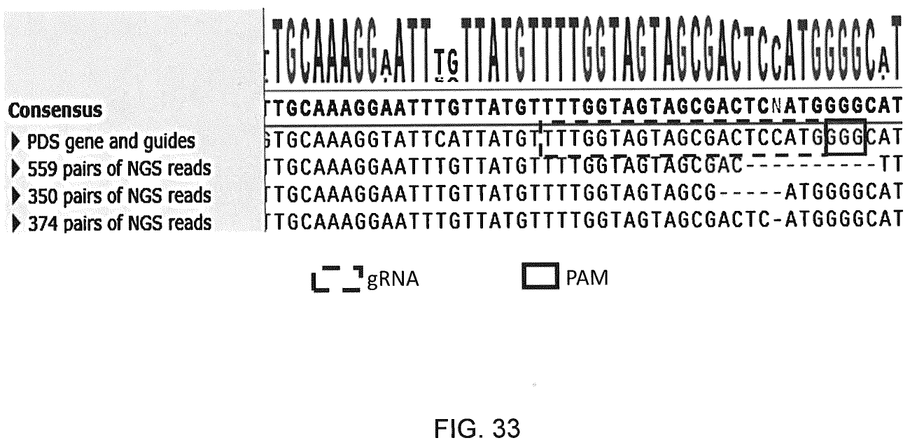

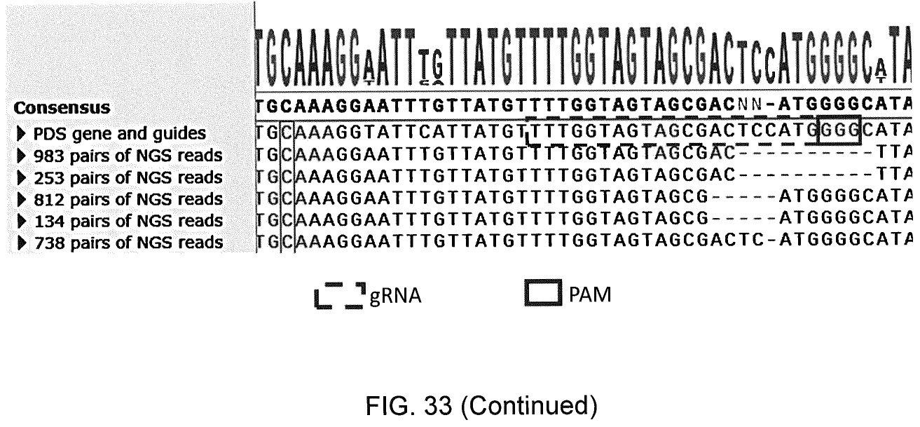

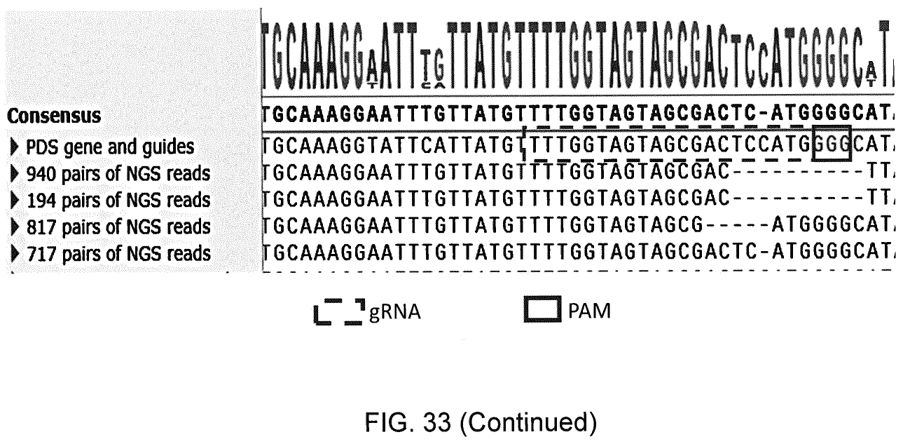

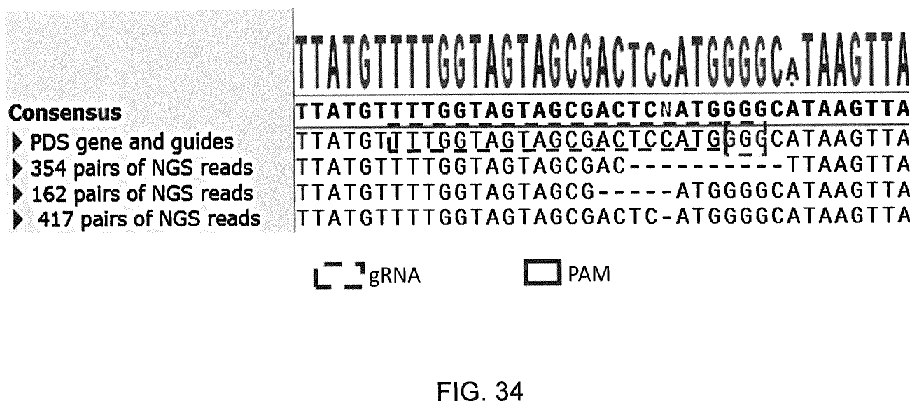

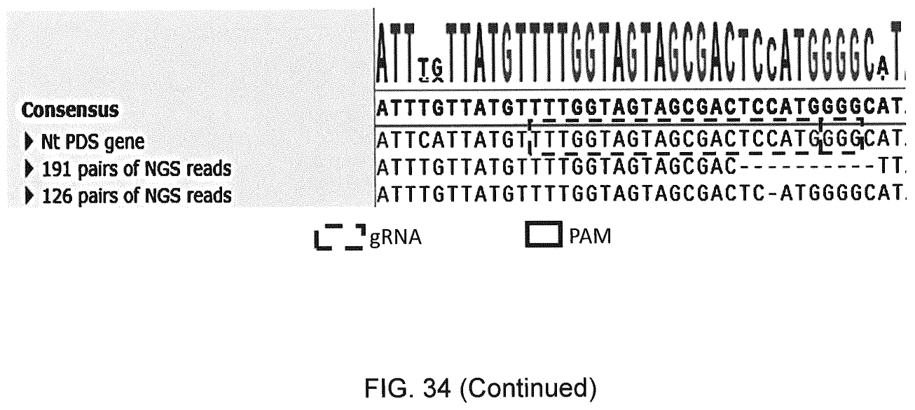

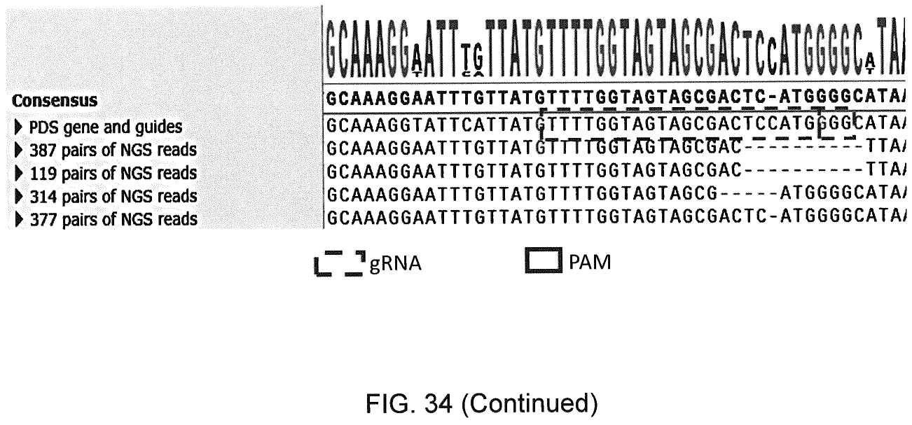

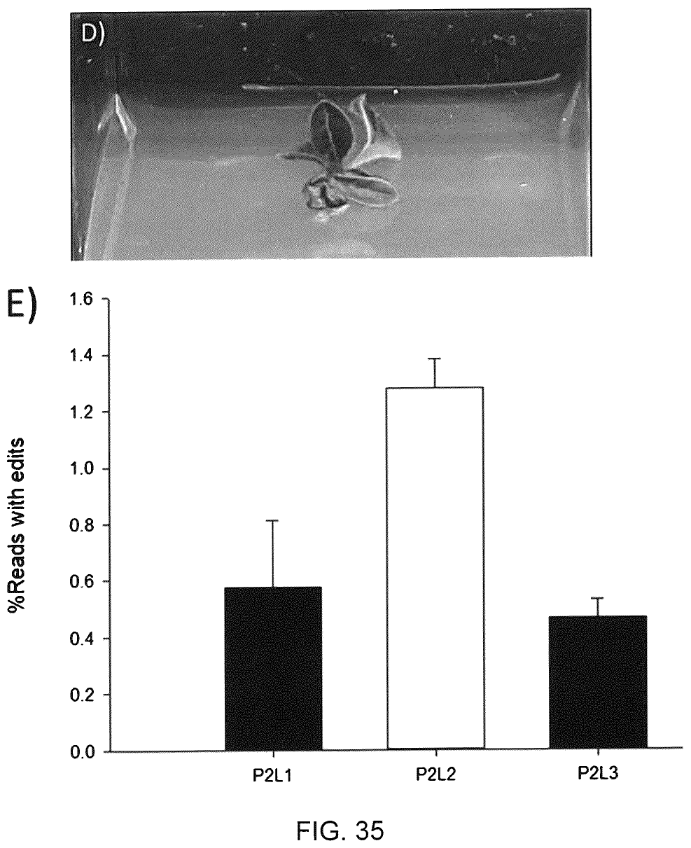

Three independent leaves from WT scion grafted on Nt-pFRG27+pFGR32-7 were placed on leaf regeneration media (MS media supplemented with 1.0 mg/L BAP, 0.2 mg/L NAA, 0.7% Agar, pH 5.8) individually. This resulted in regeneration of green shoots, which turned albino after 8 weeks (FIG. 16). The presence of NptII gene was confirmed in the regenerated shoots using PCR, and edits in the NtPDS gene were confirmed using Illumina sequencing (FIGS. 17-19). These results demonstrate the transmission of gene editing components and maintenance of the edits even after regenerating new plants via a somatic process. Interestingly, when the leaf explants from WT scion grafted on Nt-pFRG27+pFGR32-7 were regenerated on leaf regeneration media supplemented with 100 mg/L Kanamycin, albino regenerants were obtained while chimeric and albino regenerants were obtained in the leaf regeneration media without Kanamycin (FIG. 23). Edits were confirmed using Illumina sequencing (FIGS. 24-35). This is believed to be the first demonstration of regenerating plants on a selection media where the selectable marker gene is not stably integrated into the genome of the plant but is being expressed from a transcriptional unit in a virus.

Furthermore, the results described herein above demonstrate that transgenic lines expressing the Crispr/Cas9/gRNA machinery cloned within TRV RNA1 and RNA2 produce gene editing at the same efficiency as a control vector and that a transgenic line expressing these components successfully delivered the entire gene editing complex across the graft junction. Further, the edits were maintained in the somatic generation obtained via organogenesis.

### Example 5. Inter-Species Grafting for Delivery of Gene Editing Reagents

To evaluate the movement of TRV1 and TRV2-CRISPR/Cas9, and gRNA across the graft junction into other plant species, the transgenic tobacco line described above was used as a scion to graft over seven different wildtype plant species as rootstock namely, Apple (Malus x domestica M11IL659), Pear (Pyrus communis OHXF97C658), Grape (Vitis vinifera Paulsen C709), Cherry (Prunus avium Gi12-C656), Blueberry (Vaccinium sect. Cyanoccocus Elliot L695), Tomato (Solanum lycopersicum cv Heinz) and Strawberry (Fragaria x ananassa Totem C683). Four weeks post-grafting, three leaf tissue was harvested from each WT rootstock, and total cellular DNA was isolated using DNeasy plant kit (Qiagen Inc.). PCR amplification was performed to detect Cas9, TRV RNA 1, RNA2 and NtPDS-gRNA using primers listed previously. RNA was extracted using the CTAB extraction method (Gasic et al., 2004). cDNA was synthesized using Super Script VILO IV (Thermofisher Scientific® #11756050) PCR was performed using cDNA as a template to verify if the viral genes were transcribed. The primers used targeting TRV1 (FGRP273 and FGRP274; FGRP263 and FGRP264), TRV2-CRISPR/Cas9 (FGRP257 and FGRP258; FGRP253 and FGRP254; FGRP259 and FGRP260; FGRP261 and FGRP262), and gRNA (RCWP374 and RWCP375) are shown in Table 4.

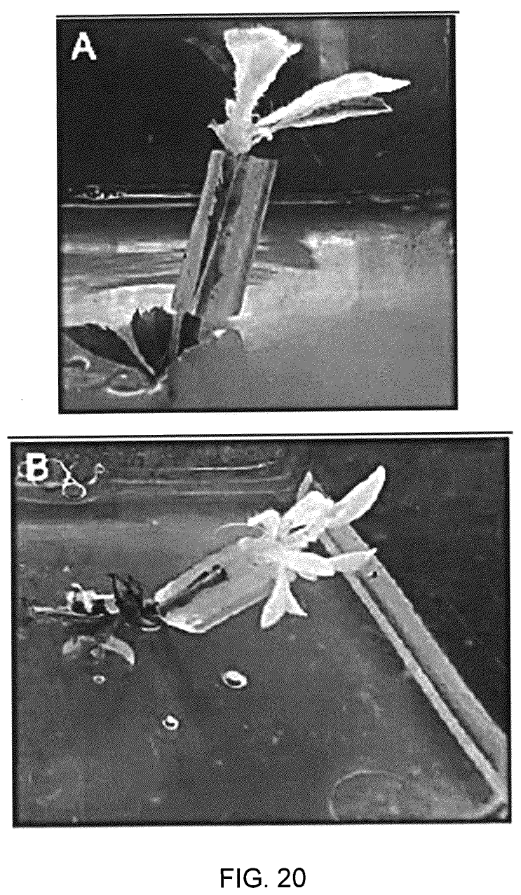

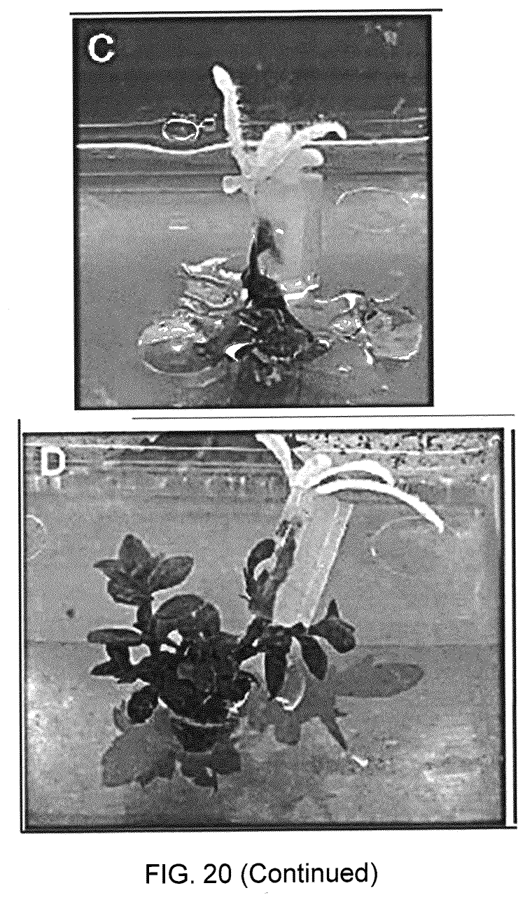

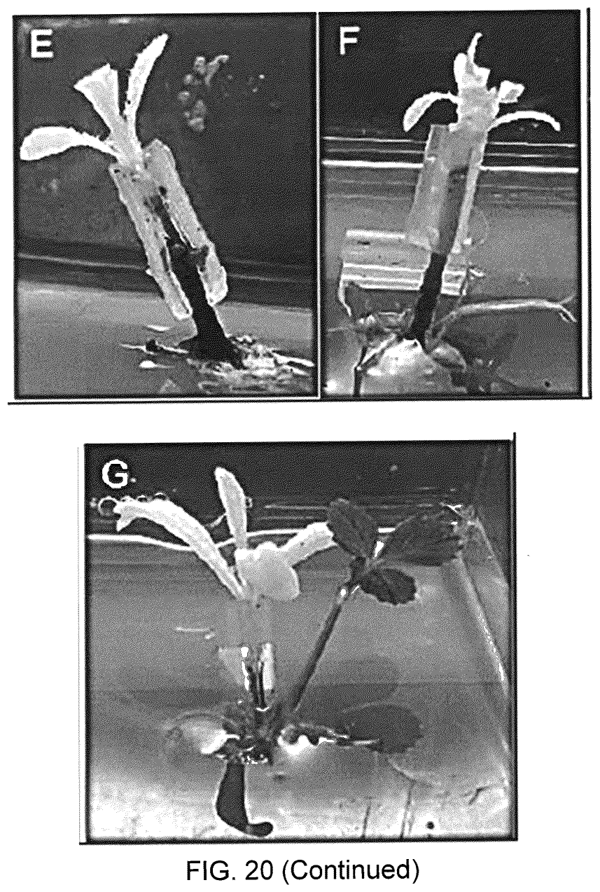

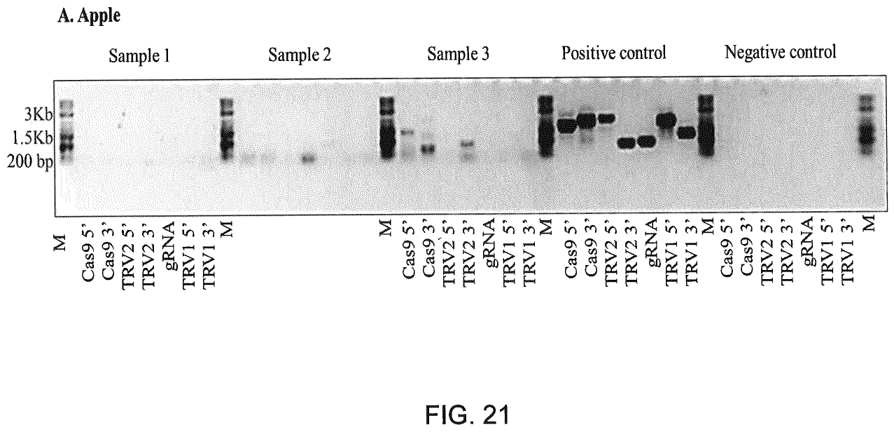

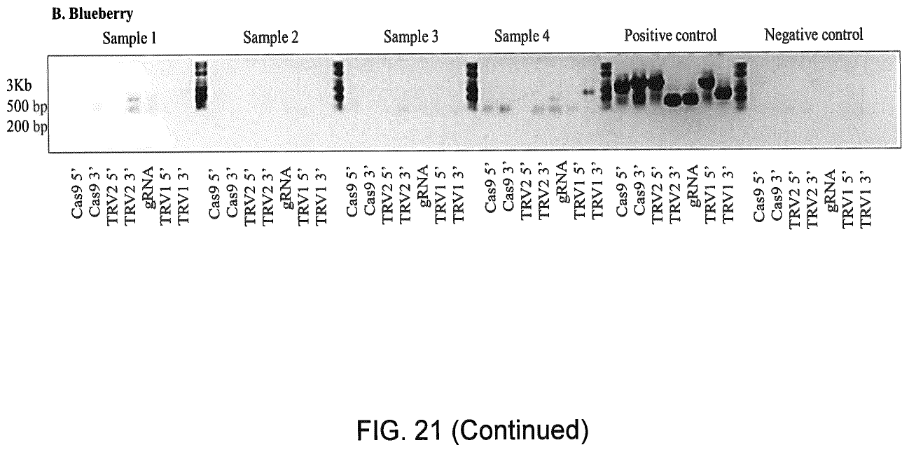

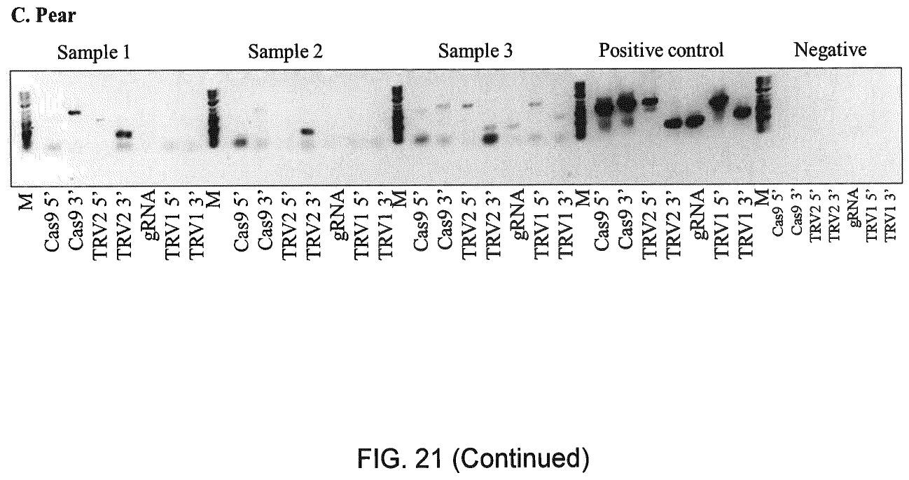

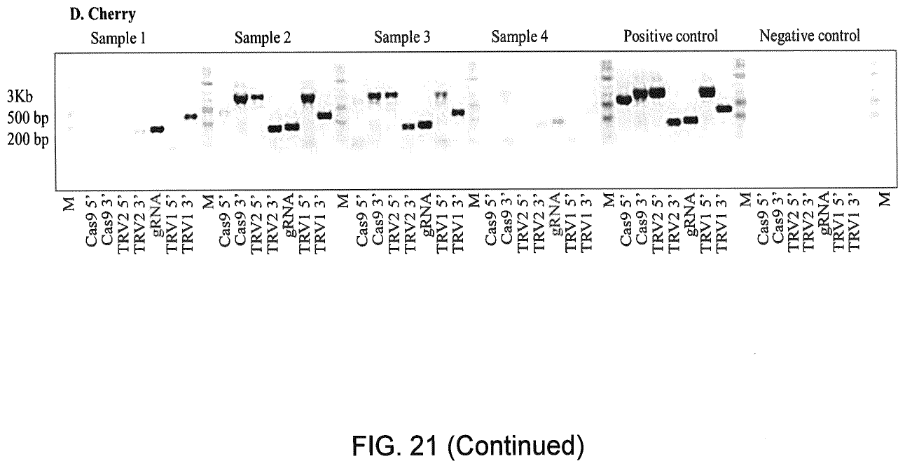

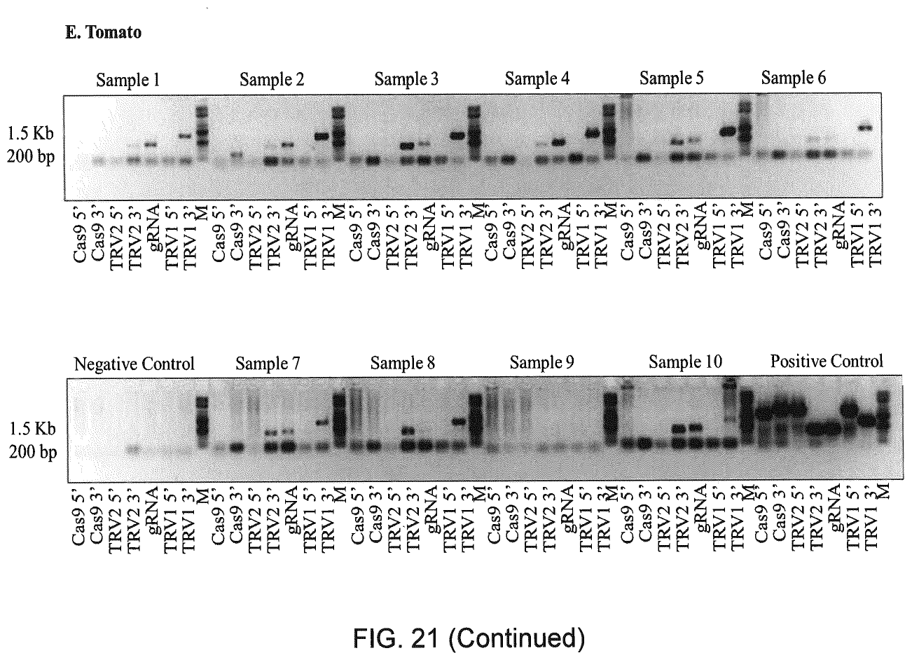

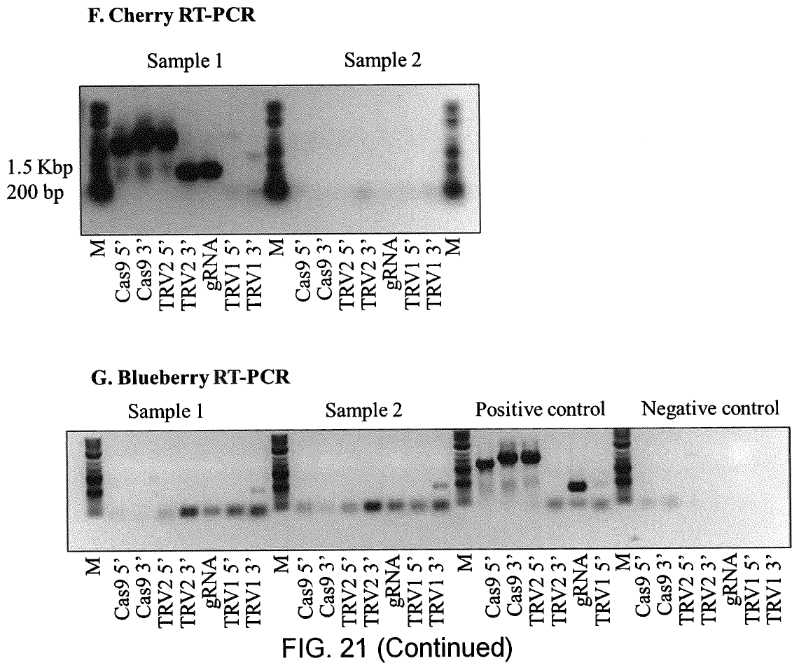

Complete delivery of gene editing reagents across the graft junction as provided herein, especially with tobacco as a donor of the reagents, is highly advantageous since the diploid N. benthamiana has been shown to be cross-graft compatible with multiple plant species. In particular, N. tabacum was used as a donor of gene editing reagents to wild-type lines of sweet cherry, blueberry, tomato, apple, and pear (FIG. 20). Using PCR, transmission of TRV RNA1 and RNA2 and the gene editing reagents was confirmed across inter-species graft junctions between tobacco and sweet cherry, blueberry, tomato, apple, and pear (FIG. 21). cDNA was also evaluated to verify the presence and expression of the virus and the gene editing reagents. The movement of TRV and gene editing reagents was confirmed in sweet cherry and blueberry.

### Example 6: Design and Use of the Additional Constructs for Delivery of Gene Editing Reagents

Additional gene editing constructs using vectors, e.g. from Tobacco Rattle Virus (TRV), Bean Yellow Dwarf Virus (BeYDV), Wheat Dwarf Virus (WDV), Tomato Mosaic Virus (ToMV), Pea Browning Virus (PEBV), Sonchus Yellow Net Rhabdovirus (SYNV), Potato Virus X (PVX), Foxtail Mosaic Virus (FoMV), Barley Yellow Striate Mosaic Virus (BYSMV), and Beet Necrotic Yellow Vein Virus (BNYVV) will be designed for modifying a plant genome analogous to those described in Example 1. These constructs will be tested for their ability to deliver gene editing reagents across graft junctions; and their ability to generate genomic modifications in plants and seeds. Based on the results disclosed herein, it is expected that additional viruses will result in similar or increased editing efficiency, and will be capable of generating homozygous edits in diploid and polyploid plants using tobacco as a donor rootstock.

Having described the present disclosure in detail, it will be apparent that modifications, variations, and equivalent embodiments are possible without departing from the spirit and scope of the present disclosure as described herein and in the appended claims. Furthermore, it should be appreciated that all examples in the present disclosure are provided as non-limiting examples.

1. A method for modifying a plant genome, the method comprising:
a) obtaining a transgenic plant comprising a recombinant DNA construct encoding a site-specific endonuclease operably linked to a viral protein, or a rootstock therefrom;
b) grafting a scion to said transgenic plant or rootstock therefrom, wherein the scion lacks said recombinant DNA construct; and
c) selecting at least a first cell from said scion comprising a genomic modification resulting from the presence of said site-specific endonuclease.

2. The method of claim 1, wherein the method further comprises generating a plant comprising the genomic modification from at least the first cell from said scion.

3. The method of claim 2, wherein generating occurs;
in the presence of a selection agent; or
by organogenesis or somatic embryogenesis.

4. (canceled)

5. The method of claim 3, wherein organogenesis produces a root, a shoot, or somatic embryo comprising the genomic modification.

6. The method of claim 1, wherein the recombinant DNA construct:
further comprises a sequence encoding a plant selectable marker;
is stably integrated into the genome of the transgenic plant; or
further comprises a sequence capable of expressing at least one guide RNA sequence (gRNA).

7. (canceled)

8. (canceled)

9. The method of claim 1, wherein the transgenic plant further comprises a second recombinant DNA construct encoding:
i) at least one viral replication protein;
ii) a movement protein; or
iii) a combination thereof.

10. The method of claim 9, wherein the second recombinant DNA construct:
encodes a viral protein from Tobacco Rattle Virus (TRV), Cabbage Leaf Curl Virus (CaLCuV), Bean Yellow Dwarf Virus (BeYDV), Pea early browning virus (PEBV), Tomato Mottle Mosaic Virus (TMMV), Bean Golden Mosaic Virus (BGMV), Tomato golden mosaic virus (TGMV), Cucumber Mosaic virus (CMV); or
comprises a sequence encoding a protein having at least 85%, 90%, 95%, or 98% identity to a polypeptide selected from the group consisting of SEQ ID NO:43, SEQ ID NO: 44, and SEQ ID NO:45.

11. (canceled)

12. The method of claim 1, wherein the recombinant DNA construct encodes a viral protein from Tobacco Rattle Virus (TRV), Bean Yellow Dwarf Virus (BeYDV), Wheat Dwarf Virus (WDV), Tomato Mosaic Virus (ToMV), Pea Browning Virus (PEBV), Sonchus Yellow Net Rhabdovirus (SYNV), Potato Virus X (PVX), Foxtail Mosaic Virus (FoMV), Barley Yellow Striate Mosaic Virus (BYSMV), Beet Necrotic Yellow Vein Virus (BNYVV).

13. The method of claim 1, wherein:
the genomic modification is selected from the group consisting of a substitution, an insertion, an inversion, a deletion, a duplication, a transposition, and a combination thereof;
the genomic modification comprises introducing a heterologous DNA molecule into the plant genome;
the transgenic plant is a N. tabacum plant;
the transgenic plant and the scion are non-isogenic;
the transgenic plant and the scion are of the same plant species; or
the transgenic plant and the scion are of different plant species.

14. (canceled)

15. The method of claim 13, wherein the heterologous DNA molecule comprises a transcribable DNA sequence operably linked to a plant-expressible promoter.

16-19. (canceled)

20. The method of claim 1, wherein the site-specific endonuclease:
i) is selected from the group consisting of: an RNA-guided nuclease, a zinc-finger nuclease, a meganuclease, a TALE-nuclease, a recombinase, a transposase, and combinations of any thereof;
ii) is an RNA-guided nuclease comprising a Cas nuclease, a Cpf1 nuclease, or a variant of either thereof; or
iii) comprises a sequence having at least 85%, 90%, 95%, or 98% identity to a polypeptide selected from the group consisting of SEQ ID NOs: 37, 39, 40, and 41.

21. The method of claim 1, wherein the genomic modification is:
i) in a transcribable region of the genome; or
ii) in a non-transcribable region of the genome.

22. The method of claim 1, wherein the genomic modification confers an altered phenotype as compared to the phenotype of an otherwise isogenic plant that lacks the modification.

23. A recombinant DNA construct encoding a site-specific endonuclease operably linked to a protein having at least 85%, 90%, 95%, or 98% identity to a polypeptide selected from the group consisting of SEQ ID NO:42 and SEQ ID NO:46.

24. The recombinant DNA construct of claim 23, wherein the sequence is stably integrated into the genome of a plant.

25. A method for producing a transgenic plant cell, comprising transforming a plant cell with at least one DNA molecule or vector comprising the recombinant DNA construct of claim 23 to produce one or more transformed plant cells comprising the recombinant DNA construct stably integrated into the genome of the one or more transformed plant cells.

26. The method of claim 25, wherein:
i) said plant cell is transformed via Agrobacterium-mediated transformation or Rhizobium-mediated transformation;
ii) said plant cell is transformed via microprojectile-mediated transformation or particle bombardment-mediated transformation; or
iii) said transgenic plant cell is tobacco plant cell.

27. A method of regenerating a plant, the method comprising:
a) obtaining at least a first cell from a plant comprising a recombinant DNA construct encoding a plant selectable marker gene operably linked to a viral protein, wherein the recombinant DNA construct is not stably integrated into at least the first cell; and
b) regenerating a plant comprising the recombinant DNA construct from at least the first cell in the presence of a selection agent.

28. The method of claim 27, wherein:
the selectable marker gene is an antibiotic resistance gene, a herbicide resistance gene, or a metabolic resistance gene; or
regenerating occurs by organogenesis or somatic embryogenesis.

29. (canceled)


SEQUENCE LISTING (46 sequences)

SEQ ID NO: 1 - DNA, 18419 bp, synthetic construct
  source 1..18419: mol_type = other DNA
cagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagaggatcgatccgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgtgtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctggagttcattcagggcaccggacaggtcggtcttgacaaaaagaaccgggcgcccctgcgctgacagccgaaacacggcggcatcagagcagccgattgtctgttgtgcccagtcatagccgaatagcctctccacccaagcggccggagaacctgcgtgcaatccatcttgttcaatccccatggtcgatcgacagatctgcgaaagctcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacggggatcctctagagtcgacctgcaggcatgcaagcttgcatgcctgcaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggataaaacattgcacctatggtgttgccctggctggggtatgtcagtgatcgcagtagaatgtactaattgacaagttggagaatacggtagaacgtccttatccaacacagcctttatccctctccctgacgaggtttttgtcagtgtaatatttctttttgaactatccagcttagtaccgtacgggaaagtgactggtgtgcttatctttgaaatgttactttgggtttcggttctttaggttagtaagaaagcacttgtcttctcatacaaaggaaaacctgagacgtatcgcttacgaaagtagcaatgaaagaaaggtggtggttttaatcgctaccgcaaaaacgatggggtcgttttaattaacttctcctacgcaagcgtctaaacggacgttggggttttgctagtttctttagagaaaactagctaagtctttaatgttatcattagagatggcataaatataatacttgtgtctgctgataagatcattttaatttggacgattagacttgttgaactacaggttactgaatcacttgcgctaatcaacatgggagatatgtacgatgaatcatttgacaagtcgggcggtcctgctgacttgatggacgattcttgggtggaatcagtttcgtggaaagatctgttgaagaagttacacagcataaaatttgcactacagtctggtagagatgagatcactgggttactagcggcactgaatagacagtgtccttattcaccatatgagcagtttccagataagaaggtgtatttccttttagactcacgggctaacagtgctcttggtgtgattcagaacgcttcagcgttcaagagacgagctgatgagaagaatgcagtggcgggtgttacaaatattcctgcgaatccaaacacaacggttacgacgaaccaagggagtactactactaccaaggcgaacactggctcgactttggaagaagacttgtacacttattacaaattcgatgatgcctctacagctttccacaaatctctaacttcgttagagaacatggagttgaagagttattaccgaaggaactttgagaaagtattcgggattaagtttggtggagcagctgctagttcatctgcaccgcctccagcgagtggaggtccgatacgtcctaatccctagggatttaaggacgtgaactctgttgagatctctgtgaaattcagagggtgggtgataccatattcactgatgccattagcgacatctaaatagggctaattgtgactaatttgagggaatttcctttaccattgacgtcagtgtcgttggtagcatttgagtttcgcaatgcacgaattacttaggaagtggcttgacgacactaatgtgttattgttagataatggtttggtggtcaaggtacgtagtagagtcccacatattcgcacgtatgaagtaattggaaagttgtcagtttttgataattcactgggagatgatacgctgtttgagggaaaagtagagaacgtatttgtttttatgttcaggcggttcttgtgtgtcaacaaagatggacattgttactcaaggaagcacgatgagctttattattacggacgagtggacttagattctgtgagtaaggttaccgaattcgttgtcaatcaattggcaagtcataaaatgcattaaaaaatattttcatactcaactacaaatccatgagtataactataattataaagcaatgattagaatctgacaaggattctggaaaattacataaaggaaagttcataaatgtctaaaacacaagaggacatacttgtattcagtaacatttgcagcttttctaggtctgaaaatatatttgttgcctagtgaataagcataatggtacaactacaagtgttttactcctcatattaacttcggtcattagaggccacgatttgacacatttttactcaaaacaaaatgtttgcatatctcttataatttcaaattcaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatactcaaactcagtaggattctggtgtgtgcgcaatgaaactgatgcattgaacttgacgaacgttgtcgaaaccgatgatacgaacgaaagctctgagctcacttcttcttcttcgcctgccccgccttcttcgtcgccgctggccgcttgtcgcccccgagctgagacaggtcgatgcgcgtctcgtagaggccggtaatcgactggtggatgagggtcgcgtccaggacctccttagtgcttgtgtacctcttgcgatcgatagttgtgtcgaagtacttgaaagcagcaggggcgccgaggttcgtcagggtgaagagatgaatgatattctcagcctgctccctgattggcttgtcgcggtgcttgttgtacgcggagaggaccttatccagattcgcgtcggccaggatcacgcgcttggagaactcggaaatctgctcaatgatctcgtcgaggtaatgcttgtgctgctcgacgaacagctgcttctgctcgttgtcctcggggctgcccttgagcttctcgtagtgggaggccaggtagaggaagttcacatacttggacggcagagccagctcgttccccttctgcagctcgccagcggaagccagcatccgcttcctgccgttctccagctcgaagagtgagtacttggggagcttaatgatcaggtccttcttcacctccttgtagcccttcgcctccaggaaatcgatcgggttcttctcgaagctggagcgctccataatcgtgatccccagcagctccttcacgctcttgagcttcttggacttgcccttctcaaccttcgccacaaccaggaccgagtaggccacagtggggctgtcgaacccgccgtacttcttcggatcccagtccttcttgcgggcgatgagcttgtcgctgttccgcttaggcagaattgactccttagagaacccgccagtctggacctctgtcttcttgacgatattcacttgtggcatggagagaaccttcctgacggtcgcgaaatccctgcccttgtcccacacgatctcccccgtctcgccgttcgtctcgatgagggggcgcttccggatctcgccattggccagagtgatctctgtcttgaagaaattcataatgttagagtagaagaagtacttggcggtagccttgccaatctcctgctccgacttggcgatcatcttcctcacatcgtaaaccttgtagtccccgtacacgaactcgctctcgagctttgggtacttcttgatcagagctgtgccgaccaccgcgttcaggtacgcgtcatgggcatggtggtaattgttgatctcccgaaccttgtagaactggaaatccttcctgaagtcggagacgagctttgacttcagggtgatgaccttcacctcgcggatcagcttgtcattctcatcgtacttagtgttcatccgtgagtcgagaatctgcgcaacgtgcttagtgatctgccgtgtctcgaccagctgcctcttgatgaagcccgccttgtccagctcagagagcccgcccctctcagcctttgtgaggttatcgaacttccgctgcgtgatcagcttggcattcaggagctggcgccagtagttcttcatcttcttaacgacctcctctgaaggaacattatcagacttgccccggttcttgtccgacctggtgaggaccttgttgtcaatggagtcatccttcaggaatgactgtggaacgatatgatcgacgtcgtaatcgctgagcctgttaatatccagctcctggtccacatacatatcgcggccattctggaggtagtacaggtagagcttctcattctgcagctgcgtgttctccaccgggtgctccttgaggatctgggaccccagctccttaatgccctcctcgatcctcttcatcctctcgcgtgagttcttctggcccttctgcgtggtctgattctcccgggccatctcaatgacgatgttctcaggcttgtgcctgcccatgaccttcaccagctcgtccacaaccttcacggtctgcagaatccccttcttgatagctggcgagccagcgaggttcgcgatatgctcgtgcagcgagtccccctggccgctcacctgagccttctggatatcctccttgaatgtgaggctgtcatcgtgaatcagctgcatgaaattgcggttcgcgaagccatcgctcttcaggaagtcgaggatcgtcttcccggactgcttgtcccgaatgccgttgatgagcttcctgctcagcctcccccagccggtgtacctcctcctcttgagctgcttcatgaccttgtcatcgaagagatgggcgtaagtcttcaggcgctcctcgatcatctcccggtcctcgaacagagtgagtgtcagcacaatgtcctcgaggatatcctcattctcctcgttgtccaggaagtccttatccttaatgatcttcaggagatcgtggtaggtccccagggaggcgttgaagcggtcctcaacgccagagatctcgaccgaatcgaagcactcaatcttcttgaagtagtcctccttgagctgcttaaccgtgaccttccggttggtcttgaacaggaggtccacgatggccttcttctgctccccagacaggaaagccggcttcctcatgccctcggtcacatacttcaccttagtcagctcgttgtagactgtgaagtactcgtacaggagcgagtgcttagggagcaccttctcatttggcaggttcttgtcgaaattcgtcatcctctcgatgaacgactgagcgctagcgcccttgtcgaccacctcctcgaagttccacggcgtgatcgtctcctctgacttgcgggtcatccaagcgaagcgggagttgcccctagcgagtgggccgacgtagtacgggatcctgaaagtcagaatcttctcgatcttctcgcggttatccttgaggaaagggtagaagtcctcctgcctcctcaggatagcgtgcagctccccgagatgaatctggtgtgggatgctgccgttatcgaatgtccgctgcttcctcaggaggtcctcgcgattgagcttcaccagcagctcctccgtgccgtccatcttctccagaatcggcttgatgaacttgtagaactcctcctgagaggccccgccgtcaatgtacccagcgtagccgttcttcgactgatcgaagaagatctccttgtacttctcggggagctgctgcctgaccagcgccttcaggagggtcagatcctgatggtgctcgtcgtagcgcttgatcatggaggctgagagcggagccttcgtaatctcggtgttcaccctgagaatatcagacaggaggatggcgtccgacagattcttggcagcgaggaacaggtccgcgtactgatcgccgatctgggccaggaggttatccaggtcatcgtcgtatgtgtccttggagagctgcagcttggcgtcctcagcgagatcgaaattcgacttgaagttgggcgtgagccccaggctgagcgcaatgagattcccgaacaggccgttcttcttctcgcccggcagctgggcgatcaggttctcgaggcgccgagacttcgagagcctagcggacaggatagccttcgcgtcgacgcctgacgcattaatggggttctcctcgaagagctggttgtacgtctgcacgagctggatgaacagcttgtcaacatcgctattgtccgggttgagatccccctcgatcaggaaatggcccctgaacttaatcatgtgggccagagcgaggtagatcaggcggaggtccgccttatctgtggagtccacgagcttcttccgcagatggtagatcgtagggtacttctcgtggtaggcaacctcgtcgacaatgttgccgaagattggatgccgctcgtgcttcttatcctcctccacgaggaatgactcctccagcctgtggaagaaagaatcgtcaaccttcgccatctcgttggagaaaatctcctgcaggtagcagatgcgattcttcctgcgcgtgtaccgcctgcgggcggtgcgcttgagccgcgtagcctcagccgtctcgccgctgtcgaacaggagagcgccaatgagattcttcttgatggaatgccgatcggtgttgcccaggaccttgaacttctttgagggcaccttgtactcgtcggtgatcacggcccagccaacagagttagtcccaatatcgaggccgatcgagtacttcttgtcagcagctggcaccccgtggatgccaaccttcctcttcttcttcggagccatcttgtcatcatcatccttgtaatcaatgtcgtggtccttgtaatccccgtcgtggtccttgtaatccatctagattactcgagtatcgttcgtaaatggtgaaaattttcagaaaattgcttttgctttaaaagaaatgatttaaattgctgcaatagaagtagaatgcttgattgcttgagattcgtttgttttgtatatgttgtgttgaggtcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacctgcaggcatgcaagcttattggtttatctcatcggaactgcaaaactcaactaactgaaaagtacaaaaccactgaatcatattatttgagattttttttaggtcaaattttaggtttcagttacagaaaacgaagagaaaaccccagaaattgaacgccgaagaacagaggaagaagaaatcgatctggaaaattttgcaaaaaaaagcaccgactcggtgccactttttcaagttgataacggactagccttattttaacttgctatttctagctctaaaaccatggagtcgctactaccaacaatcactacttcgactctagctgtatataaactcagcttcgttttcttatctaagcgatgtgggacttttgaagattgttttcaacttaaatgggcctatataagaaatactattgttctttcccatataaatgggcctgcttctcttctttcagattcccaggggccttttgaagattatcttcatatcttaagaatgaagatgttttattcaatcaaattcttgaaggttcgatgcctaatcattctaatcctgggacaaactatgaaacaagatacaaaaactccgaatggaaagttaaaaagaagaaaacgaaagctacggttcaagaaaatgtaagctgataaacaaaaaaaaactgtatgaacgaagaagaagaaaaaaagctaagaagaaatgatgtattgtgcggaaggcaagtcgaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttgggcatgtcccgaagacattaaactacggttctttaagtagatccgtgtctgaagttttaggttcaatttaaacctacgagattgacattctcgactgatcttgattgatcggtaagtcttttgtaatttaattttctttttgattttattttaaattgttatctgtttctgtgtatagactgtttgagatcggcgtttggccgactcattgtcttaccataggggaacggactttgtttgtgttgttattttatttgtattttattaaaattctcaacgatctgaaaaagcctcgcggctaagagattgttggggggtgagtaagtacttttaaagtgatgatggttacaaaggcaaaaggggtaaaacccctcgcctacgtaagcgttattacgcccgtctgtacttatatcagtacactgacgagtccctaaaggacgaaacgggagaacgctagccaccaccaccaccaccacgtgtgaattacaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtgtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaa

SEQ ID NO: 2 - DNA, 17701 bp, synthetic construct
  source 1..17701: mol_type = other DNA
agccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagaggatcgatccgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgtgtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctggagttcattcagggcaccggacaggtcggtcttgacaaaaagaaccgggcgcccctgcgctgacagccgaaacacggcggcatcagagcagccgattgtctgttgtgcccagtcatagccgaatagcctctccacccaagcggccggagaacctgcgtgcaatccatcttgttcaatccccatggtcgatcgacagatctgcgaaagctcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacggggatcctctagagtcgacctgcaggcatgcaagcttgcatgcctgcaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggataaaacattgcacctatggtgttgccctggctggggtatgtcagtgatcgcagtagaatgtactaattgacaagttggagaatacggtagaacgtccttatccaacacagcctttatccctctccctgacgaggtttttgtcagtgtaatatttctttttgaactatccagcttagtaccgtacgggaaagtgactggtgtgcttatctttgaaatgttactttgggtttcggttctttaggttagtaagaaagcacttgtcttctcatacaaaggaaaacctgagacgtatcgcttacgaaagtagcaatgaaagaaaggtggtggttttaatcgctaccgcaaaaacgatggggtcgttttaattaacttctcctacgcaagcgtctaaacggacgttggggttttgctagtttctttagagaaaactagctaagtctttaatgttatcattagagatggcataaatataatacttgtgtctgctgataagatcattttaatttggacgattagacttgttgaactacaggttactgaatcacttgcgctaatcaacatgggagatatgtacgatgaatcatttgacaagtcgggcggtcctgctgacttgatggacgattcttgggtggaatcagtttcgtggaaagatctgttgaagaagttacacagcataaaatttgcactacagtctggtagagatgagatcactgggttactagcggcactgaatagacagtgtccttattcaccatatgagcagtttccagataagaaggtgtatttccttttagactcacgggctaacagtgctcttggtgtgattcagaacgcttcagcgttcaagagacgagctgatgagaagaatgcagtggcgggtgttacaaatattcctgcgaatccaaacacaacggttacgacgaaccaagggagtactactactaccaaggcgaacactggctcgactttggaagaagacttgtacacttattacaaattcgatgatgcctctacagctttccacaaatctctaacttcgttagagaacatggagttgaagagttattaccgaaggaactttgagaaagtattcgggattaagtttggtggagcagctgctagttcatctgcaccgcctccagcgagtggaggtccgatacgtcctaatccctagggatttaaggacgtgaactctgttgagatctctgtgaaattcagagggtgggtgataccatattcactgatgccattagcgacatctaaatagggctaattgtgactaatttgagggaatttcctttaccattgacgtcagtgtcgttggtagcatttgagtttcgcaatgcacgaattacttaggaagtggcttgacgacactaatgtgttattgttagataatggtttggtggtcaaggtacgtagtagagtcccacatattcgcacgtatgaagtaattggaaagttgtcagtttttgataattcactgggagatgatacgctgtttgagggaaaagtagagaacgtatttgtttttatgttcaggcggttcttgtgtgtcaacaaagatggacattgttactcaaggaagcacgatgagctttattattacggacgagtggacttagattctgtgagtaaggttaccgaattcgttgtcaatcaattggcaagtcataaaatgcattaaaaaatattttcatactcaactacaaatccatgagtataactataattataaagcaatgattagaatctgacaaggattctggaaaattacataaaggaaagttcataaatgtctaaaacacaagaggacatacttgtattcagtaacatttgcagcttttctaggtctgaaaatatatttgttgcctagtgaataagcataatggtacaactacaagtgttttactcctcatattaacttcggtcattagaggccacgatttgacacatttttactcaaaacaaaatgtttgcatatctcttataatttcaaattcaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatactcaaactcagtaggattctggtgtgtgcgcaatgaaactgatgcattgaacttgacgaacgttgtcgaaaccgatgatacgaacgaaagctctgagctcacttcttcttcttcgcctgccccgccttcttcgtcgccgctggccgcttgtcgcccccgagctgagacaggtcgatgcgcgtctcgtagaggccggtaatcgactggtggatgagggtcgcgtccaggacctccttagtgcttgtgtacctcttgcgatcgatagttgtgtcgaagtacttgaaagcagcaggggcgccgaggttcgtcagggtgaagagatgaatgatattctcagcctgctccctgattggcttgtcgcggtgcttgttgtacgcggagaggaccttatccagattcgcgtcggccaggatcacgcgcttggagaactcggaaatctgctcaatgatctcgtcgaggtaatgcttgtgctgctcgacgaacagctgcttctgctcgttgtcctcggggctgcccttgagcttctcgtagtgggaggccaggtagaggaagttcacatacttggacggcagagccagctcgttccccttctgcagctcgccagcggaagccagcatccgcttcctgccgttctccagctcgaagagtgagtacttggggagcttaatgatcaggtccttcttcacctccttgtagcccttcgcctccaggaaatcgatcgggttcttctcgaagctggagcgctccataatcgtgatccccagcagctccttcacgctcttgagcttcttggacttgcccttctcaaccttcgccacaaccaggaccgagtaggccacagtggggctgtcgaacccgccgtacttcttcggatcccagtccttcttgcgggcgatgagcttgtcgctgttccgcttaggcagaattgactccttagagaacccgccagtctggacctctgtcttcttgacgatattcacttgtggcatggagagaaccttcctgacggtcgcgaaatccctgcccttgtcccacacgatctcccccgtctcgccgttcgtctcgatgagggggcgcttccggatctcgccattggccagagtgatctctgtcttgaagaaattcataatgttagagtagaagaagtacttggcggtagccttgccaatctcctgctccgacttggcgatcatcttcctcacatcgtaaaccttgtagtccccgtacacgaactcgctctcgagctttgggtacttcttgatcagagctgtgccgaccaccgcgttcaggtacgcgtcatgggcatggtggtaattgttgatctcccgaaccttgtagaactggaaatccttcctgaagtcggagacgagctttgacttcagggtgatgaccttcacctcgcggatcagcttgtcattctcatcgtacttagtgttcatccgtgagtcgagaatctgcgcaacgtgcttagtgatctgccgtgtctcgaccagctgcctcttgatgaagcccgccttgtccagctcagagagcccgcccctctcagcctttgtgaggttatcgaacttccgctgcgtgatcagcttggcattcaggagctggcgccagtagttcttcatcttcttaacgacctcctctgaaggaacattatcagacttgccccggttcttgtccgacctggtgaggaccttgttgtcaatggagtcatccttcaggaatgactgtggaacgatatgatcgacgtcgtaatcgctgagcctgttaatatccagctcctggtccacatacatatcgcggccattctggaggtagtacaggtagagcttctcattctgcagctgcgtgttctccaccgggtgctccttgaggatctgggaccccagctccttaatgccctcctcgatcctcttcatcctctcgcgtgagttcttctggcccttctgcgtggtctgattctcccgggccatctcaatgacgatgttctcaggcttgtgcctgcccatgaccttcaccagctcgtccacaaccttcacggtctgcagaatccccttcttgatagctggcgagccagcgaggttcgcgatatgctcgtgcagcgagtccccctggccgctcacctgagccttctggatatcctccttgaatgtgaggctgtcatcgtgaatcagctgcatgaaattgcggttcgcgaagccatcgctcttcaggaagtcgaggatcgtcttcccggactgcttgtcccgaatgccgttgatgagcttcctgctcagcctcccccagccggtgtacctcctcctcttgagctgcttcatgaccttgtcatcgaagagatgggcgtaagtcttcaggcgctcctcgatcatctcccggtcctcgaacagagtgagtgtcagcacaatgtcctcgaggatatcctcattctcctcgttgtccaggaagtccttatccttaatgatcttcaggagatcgtggtaggtccccagggaggcgttgaagcggtcctcaacgccagagatctcgaccgaatcgaagcactcaatcttcttgaagtagtcctccttgagctgcttaaccgtgaccttccggttggtcttgaacaggaggtccacgatggccttcttctgctccccagacaggaaagccggcttcctcatgccctcggtcacatacttcaccttagtcagctcgttgtagactgtgaagtactcgtacaggagcgagtgcttagggagcaccttctcatttggcaggttcttgtcgaaattcgtcatcctctcgatgaacgactgagcgctagcgcccttgtcgaccacctcctcgaagttccacggcgtgatcgtctcctctgacttgcgggtcatccaagcgaagcgggagttgcccctagcgagtgggccgacgtagtacgggatcctgaaagtcagaatcttctcgatcttctcgcggttatccttgaggaaagggtagaagtcctcctgcctcctcaggatagcgtgcagctccccgagatgaatctggtgtgggatgctgccgttatcgaatgtccgctgcttcctcaggaggtcctcgcgattgagcttcaccagcagctcctccgtgccgtccatcttctccagaatcggcttgatgaacttgtagaactcctcctgagaggccccgccgtcaatgtacccagcgtagccgttcttcgactgatcgaagaagatctccttgtacttctcggggagctgctgcctgaccagcgccttcaggagggtcagatcctgatggtgctcgtcgtagcgcttgatcatggaggctgagagcggagccttcgtaatctcggtgttcaccctgagaatatcagacaggaggatggcgtccgacagattcttggcagcgaggaacaggtccgcgtactgatcgccgatctgggccaggaggttatccaggtcatcgtcgtatgtgtccttggagagctgcagcttggcgtcctcagcgagatcgaaattcgacttgaagttgggcgtgagccccaggctgagcgcaatgagattcccgaacaggccgttcttcttctcgcccggcagctgggcgatcaggttctcgaggcgccgagacttcgagagcctagcggacaggatagccttcgcgtcgacgcctgacgcattaatggggttctcctcgaagagctggttgtacgtctgcacgagctggatgaacagcttgtcaacatcgctattgtccgggttgagatccccctcgatcaggaaatggcccctgaacttaatcatgtgggccagagcgaggtagatcaggcggaggtccgccttatctgtggagtccacgagcttcttccgcagatggtagatcgtagggtacttctcgtggtaggcaacctcgtcgacaatgttgccgaagattggatgccgctcgtgcttcttatcctcctccacgaggaatgactcctccagcctgtggaagaaagaatcgtcaaccttcgccatctcgttggagaaaatctcctgcaggtagcagatgcgattcttcctgcgcgtgtaccgcctgcgggcggtgcgcttgagccgcgtagcctcagccgtctcgccgctgtcgaacaggagagcgccaatgagattcttcttgatggaatgccgatcggtgttgcccaggaccttgaacttctttgagggcaccttgtactcgtcggtgatcacggcccagccaacagagttagtcccaatatcgaggccgatcgagtacttcttgtcagcagctggcaccccgtggatgccaaccttcctcttcttcttcggagccatcttgtcatcatcatccttgtaatcaatgtcgtggtccttgtaatccccgtcgtggtccttgtaatccatctagattactcgagtatcgttcgtaaatggtgaaaattttcagaaaattgcttttgctttaaaagaaatgatttaaattgctgcaatagaagtagaatgcttgattgcttgagattcgtttgttttgtatatgttgtgttgaggtcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttgggcatgtcccgaagacattaaactacggttctttaagtagatccgtgtctgaagttttaggttcaatttaaacctacgagattgacattctcgactgatcttgattgatcggtaagtcttttgtaatttaattttctttttgattttattttaaattgttatctgtttctgtgtatagactgtttgagatcggcgtttggccgactcattgtcttaccataggggaacggactttgtttgtgttgttattttatttgtattttattaaaattctcaacgatctgaaaaagcctcgcggctaagagattgttggggggtgagtaagtacttttaaagtgatgatggttacaaaggcaaaaggggtaaaacccctcgcctacgtaagcgttattacgcccgtctgtacttatatcagtacactgacgagtccctaaaggacgaaacgggagaacgctagccaccaccaccaccaccacgtgtgaattacaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtgtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtact

SEQ ID NO: 3 - DNA, 11718 bp, synthetic construct
  source 1..11718: mol_type = other DNA
ccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagaggatcgatccgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgtgtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctggagttcattcagggcaccggacaggtcggtcttgacaaaaagaaccgggcgcccctgcgctgacagccgaaacacggcggcatcagagcagccgattgtctgttgtgcccagtcatagccgaatagcctctccacccaagcggccggagaacctgcgtgcaatccatcttgttcaatccccatggtcgatcgacagatctgcgaaagctcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacggggatcctctagagtcgacctgcaggcatgcaagcttgcatgcctgcaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggataaaacattgcacctatggtgttgccctggctggggtatgtcagtgatcgcagtagaatgtactaattgacaagttggagaatacggtagaacgtccttatccaacacagcctttatccctctccctgacgaggtttttgtcagtgtaatatttctttttgaactatccagcttagtaccgtacgggaaagtgactggtgtgcttatctttgaaatgttactttgggtttcggttctttaggttagtaagaaagcacttgtcttctcatacaaaggaaaacctgagacgtatcgcttacgaaagtagcaatgaaagaaaggtggtggttttaatcgctaccgcaaaaacgatggggtcgttttaattaacttctcctacgcaagcgtctaaacggacgttggggttttgctagtttctttagagaaaactagctaagtctttaatgttatcattagagatggcataaatataatacttgtgtctgctgataagatcattttaatttggacgattagacttgttgaactacaggttactgaatcacttgcgctaatcaacatgggagatatgtacgatgaatcatttgacaagtcgggcggtcctgctgacttgatggacgattcttgggtggaatcagtttcgtggaaagatctgttgaagaagttacacagcataaaatttgcactacagtctggtagagatgagatcactgggttactagcggcactgaatagacagtgtccttattcaccatatgagcagtttccagataagaaggtgtatttccttttagactcacgggctaacagtgctcttggtgtgattcagaacgcttcagcgttcaagagacgagctgatgagaagaatgcagtggcgggtgttacaaatattcctgcgaatccaaacacaacggttacgacgaaccaagggagtactactactaccaaggcgaacactggctcgactttggaagaagacttgtacacttattacaaattcgatgatgcctctacagctttccacaaatctctaacttcgttagagaacatggagttgaagagttattaccgaaggaactttgagaaagtattcgggattaagtttggtggagcagctgctagttcatctgcaccgcctccagcgagtggaggtccgatacgtcctaatccctagggatttaaggacgtgaactctgttgagatctctgtgaaattcagagggtgggtgataccatattcactgatgccattagcgacatctaaatagggctaattgtgactaatttgagggaatttcctttaccattgacgtcagtgtcgttggtagcatttgagtttcgcaatgcacgaattacttaggaagtggcttgacgacactaatgtgttattgttagataatggtttggtggtcaaggtacgtagtagagtcccacatattcgcacgtatgaagtaattggaaagttgtcagtttttgataattcactgggagatgatacgctgtttgagggaaaagtagagaacgtatttgtttttatgttcaggcggttcttgtgtgtcaacaaagatggacattgttactcaaggaagcacgatgagctttattattacggacgagtggacttagattctgtgagtaaggttaccgaattctctagaaggcctccatggggatccggtaccgagctcacgcgtctcgaggcccgggcatgtcccgaagacattaaactacggttctttaagtagatccgtgtctgaagttttaggttcaatttaaacctacgagattgacattctcgactgatcttgattgatcggtaagtcttttgtaatttaattttctttttgattttattttaaattgttatctgtttctgtgtatagactgtttgagatcggcgtttggccgactcattgtcttaccataggggaacggactttgtttgtgttgttattttatttgtattttattaaaattctcaacgatctgaaaaagcctcgcggctaagagattgttggggggtgagtaagtacttttaaagtgatgatggttacaaaggcaaaaggggtaaaacccctcgcctacgtaagcgttattacgcccgtctgtacttatatcagtacactgacgagtccctaaaggacgaaacgggagaacgctagccaccaccaccaccaccacgtgtgaattacaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtgtttaaactatcagtgtttgacaggatatattggcgggtaaacctaagagaaaagagcgtttattagaataatcggatatttaaaagggcgtgaaaaggtttatccgttcgtccatttgtatgtg

SEQ ID NO: 4 - DNA, 19006 bp, synthetic construct
  source 1..19006: mol_type = other DNA
cccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaagacatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgaggggcccgtttcgtcctttagggactcgtcagtgtactgatataagtacagacgggcgtaataacgcttacgtaggcgaggggttttaccccttttgcctttgtaaccatcatcactttaaaagtacttactcaccccccaataatctcttagcctcgaggccttttcagatcattgagaattttaataaaatacaaataaaataacaacacaaacaaagtccgttcccctatggtaagacaatgagtcggccaagcgccaatctcaaacagtctatacacagaaacagataacaatataaaataaaatcaaaaagcaaacaaacgatcaatcccgaaaggaacatcatctcttaaaaatctttttggtgtcccaaattctctgtcctctttgaatttcttcttagaggtcttcgaattatgagactttttagctgtcgcagacatgttttcagcttgatttcgtcgaatttgtttccaaatttctcgatttcttgtttcaacacgtttacgacacttttcaaggtgactacggccacaacaattatacacatcaaaagtaaaatcattgataacaacacagacaaaccatccacaattattttccgcacacctacgtgtgacaccaaccatgtcagcaacttgctttcgcaatttgttagcatgaccgatactacacgtctcgtgaccaagaacagtgacttcattcacacaacccttgagtacacacgtcatattgacaataaatttctttatgcattaagacgagtttttcttattaggcgctctcttaacatcttcacctgacaataatgacttaggtctagtagtagctggtctgtaaggaccatcgtacttcgcctcaatcgtcttcatctccacttgtttcaaagacatgaactcaccgaaaacctcttgatcggtaaaatcgccttcaatgtaatccgaactgttcagttcttccacattgaggcgcctagtcgaatcagtagcaacccatattagcgacacttcgatactgaaagggtatgtaccgtcacagaccggaacgccctcagaaatacaacccaccttccagacatcaagtacgttctcctgtgtggaaatactgtattcaaccttgtactgcgcaactgaacagttatcgcaagctcgagctttgaatctctgaatagtctttcctctctttcttttctcagtaagtctcgaatccaacagatcgatttgaatgaacccaggcgtatctgcaggtagatgtgatgttacaactagttgtacacctactatctttagaaacgcgtaagatttaatcattttgtaatccctttggtcaaaaacgtctaacttgtgttcactcttcttcccaccaacatgatttaggacagtttctcttcttctgtgaaaatacttcggtctcttcctcctaccgtccacaaacaattcaatggcccctagatttgagaattttgagacttcgaaagtcttcttcttcaaggtgaccaatgacttgtcttccatattaccagtcgtaaaagtttgacacagctctctgtgccttcttccaatcaaccttagccgggttcaattccttatcgttttcgtctcggaataatgtacacagagctgtaaaactcttaatatgcttccatagcgcacaaagcgcatgaacagaatcacctttgtacaaataccggtctgaaacagactcggacagtttggataccaccatgtagttcccaagagctctattggaatcattcagcgagatgtagatctcggctaaatgttgcacatcctttatactctttttccccaacttcgtcagaacttttaccggatctggcacgaactcgtaacacgatgacgtcttaagcaagaacttcccacaaaacattgggacatcgtacttaaaaatcttgcactcgaaattccacttagtagccaactttggacacggatcaacaaactgcgttcctctaggaaacgcaatcagtgagtcatctcctccatatgtaaccatgactgctttctccaatggtaattcagacaagagtgcacacagtgttctatctgaatttgcattataagtatcagcatctccactcttttgttggtaccaaatatgcgccatcataccattttgaatatctctcacagtagtttgagtgtgcgacacctcccaaaggaaggccgcccactcatctagcccaaataacctgtaaatctccagctgcagttgtaaatggaagcgatttgccgacttgtcgaatttagacatgtctatctcaacaaagtcgtaagccgcttccgtatttaagaacttcactcgatcgtttaaatcactcgatgtcattctcgtgttgaacacaaaatgtggttgcatagcatccaacttgcgttcattaatttctttgaaaattggaccgaacaacgagttgatcaacttctcgtgatacacaacagtctgtagagctgagtattcaaattgcggtgttaaatcagtcttaggtttcacgtcagatttgatcatatacatgtaagacgaatagtctatttcatgtaactctctcacatctgctactaccttagcctgtaccgctgtgctttgatttctccaccatctctccatttgagctctattgacaataggatcagcccgaatttttcccacatcgtagacaacagatttcagcttcttcatcgtctcttcgattagaactgtcgcgtgcacattttcttgtagatcgggtgccgcttggttccttttctgcaatgctaacaaactctccaactgagtagtcttccttttgtcaggcatagcagtacgaataaccggtttcagaaacgtttctttttcagcaaacttgtctttccagtttccacttttgatcgtaacattgtctaatcgcaaattgcaatcagtcgttgccaccaaatacccgtctaggcttgagtctcttaacgaatttcccggaaacaaagcgtcgtaccacatctccaagtccgtaatcgtacctgaatctggaacggcgcaaggcccttcatgatgtctaaagacatcaaacctagaccgaaaccgtcataagacggtctcagtaacaaaaaatcttgccaaagccgccttcgtaagcgcggcactttccctgatctctttgcttacatcgtcctctttcacagtttcatacacaagtgattgtgtgtgacgcgacaacgccacgattaagtactcccgacctctagcgattgagtcatccgtaggtttcgtcctgactaggactacatctttgaatgtctctccttgcgactcgtgtactgtactcactttagctgccactttcctcaatgccaacaaatttaccaactccttcttctcagactgaaggaaagttatgtactgagcaccaggcttcaaagaaacctgttctttagacacaatcttcctcaccgtcatcgaattagcagtcgcgttatgtgttctgacatctccagtatagtacttgttcaatacggcagccacatctgctggacttctgtaagtttctcttttttctgtaacaatgtcaaactttccgaccagactagaaaacctcaaatcaacttgagataccctaggcttgaaagaaatttgattctgatctccttgacagatacatcgtttagcaccagctatctgagcgcaaaagtacaccataccagcatgggccatgagagcttcatcgaaatgcaacacgtctccggttaaagaaccatcaacacaatgcatcaaaaaagaatcaaccgtcttcactctccttttcaatttgcatggaaaacctttgctcgcgaatctctcgatcaagtcgtcggttgctgctctcccagtagagagaaccacatcgacacaaggattagctgagttgacaatcattgtcgacttaccacaaccaggtacaccgtcaactaattcaaaagaacagttaactggtcctagatccagggcacttaaattgtccaagatcaacctgttattgcaaagatacgtctgatcgcaaactatgacataatcactcatcctttcgaccgcgaagtcgttgaccaacatacctcgttcccaaacagccacttctaagtatcccgaccaatcatccatacgtttgtctggttggaacacataagcatgttcagtgaccactggacgtatgcaccagttcttcatctccacatcccacactcccagtccgtgcgggtaattctgacctccagttcgaataactcctctcgaatagtcacgatacagagacataatttcatcattttgagcgactctctccgcatctaagtagtacagaaactctcttctagcgtccacagaatcgtctccactgacaaccggcctttttggtaatctctcacctcccatcacttggaagtaatcgacacgtttgaccgtttttcctttgctgaccaaaggtttgacagctggcaacacgttctcaaatcctggtggcgttaaaggtttacacaacacctttgtaccgcttgtttcccctctttcacattcagctgaaaaggctgaaacaacctcgtcaatttctgcttcagtgtcaataagaccactcagaaactgctccatgatctcctcatcgtctacctttcttgaaactccaactctagcttgggtcagcacgttactgtcttccaacaacagacccagtccgcttcgagatgaaccgaccaccgatggtggagaagttctcaccgaactgccagaatagttagagaattttgcacttagaaactcgtcagcgatttctttcttctgctgcaatagcttatcagacttggcttttacctcggataaatctggtaaaacggtctcaccggactcagatgccgaatacagactgctaaagaaatcgttgatttcataatagcggcacggattctcaacgtctaagacatgggccccccgtttgatatcaaacatatcacatagttttttcaccagtccatctcggatgtctcgacgcataagcgtgattcctccaatggcatacacaacactcagaaatactatcaccttggttttgtgcgagttctcgtaacatagttctggaccatttggtgtgtccttcgcccaggttagtaccttctttggatagaatccaattagagtaccaaccatgccaacgaatccgccgactgcgatcttgttagcaaactgcttccacagttttcttgatattaaacccagcacatcagtcaatgacgtgataggtttcgtctcagtcagaattgcttcacgcaactccctcatgacaggtctcgccacttgttccttcactagcaaagtttgagccaacaactgtaaatctcgagaatctacactttgcttgttcttcacggccgccccgtttatgaataagacccaattatttgaactcaagtatgatttaacattgcttatggtcagctgctggtcagataacctagctatgtaatccaaacacttgtccatgaattgtttctctacaaacaggtctttcttgaccaactctcgcgttgattcgaccagatcgaaaattgggacaacaaccatgttttcccatctactgatatatattcttcggtagtactcttgcgatgatagagacctcctcggaactccagctatcctgtagatcgaaaaaagcattgtgtctcctctcacctgccacggttcgaagtagaacacattcccttggtaactcactggcttccccagtagatacttcttgtactcttcccagtcatgaatgtaagaaaaggaaggatctttctcaaaaccgaaatagatcttcccgttcttcttcatgtagtaaccgtcaacagaaggtaatggaccttcgtccactaacatactttcaggagcaaaaagcatagcagcatgaaccactttagttttcttctcaaccatagtagccatcaagtctttcaacttgaagtcatacaggctgtgaagagctatcgcatactgcgcgcctttcttcttaccatcagaaaaacccctacatgtacaaccctgatatgtattattgcacctaaccgcgttaggcgcagcaatattttcacgaaattggtcatacatccacagctcattctccgacatctgtctcttaccacttctagcttgatcaccaatagcagtaagaaatatctgtcgagaaatttctctctgatcatcccgaatatctagatttggacaacaggaatgaatccttttctcacctctagttacccaaaacgaccattgaccaccgatgtcatccacctcttgcagagcaggaactctatctaaaagcaataaagcttgtagttttcgctcagcagccgcaaacccatgaaccatgtttttgtcgtctttaaacacgatattgaattcaggataaatttccatcaacttgttctgttccttctgagaaagaacttcatgaacaatcactggatctttagtctttctttctctaatcatgtcatcgactttatcaacaacaactctttgcatcatttgcccgatctcacaatcaggtttagtcagcatcaagtcaaagaaatgactcctctgctcagcagacatctcgtccacattgagcaattgagacaacttgaagttaccgttcgccattgtatctgtatgtatttttacaaagtatcaaagaaaaggttttaggcacaccagtcactttcccccactcggtactaagctggatagattaaaccaaaaagaaaaacaaaataaaactgtaggtgataagtacgttctaccgcgttctcaccaaaatccaattagcacgttctaccgcgttcaaaggattgaaatgttttatcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtagaagccatcttccttttctactgtcctttcgatgaagtgacagatagctgggcaatggaatccgaggaggtttcccgatattaccctttgttgaaaagtctcaatagccctctggtcttctgagactgtatctttgatattcttggagtagacgagagtgtcgtgctccaccatgttgaccatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtagaagccatcttccttttctactgtcctttcgatgaagtgacagatagctgggcaatggaatccgaggaggtttcccgatattaccctttgttgaaaagtctcaatagccctctggtcttctgagactgtatctttgatattcttggagtagacgagagtgtcgtgctccaccatgttgacctgcaggggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatctgtcgatcgaccatggggattgaacaagatggattgcacgcaggttctccggccgcttgggtggagaggctattcggctatgactgggcacaacagacaatcggctgctctgatgccgccgtgtttcggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggatcgatcctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacggatccatatatagggcccaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcg

SEQ ID NO: 5 - DNA, 17221 bp, synthetic construct
  source 1..17221: mol_type = other DNA
gcatgcctgcaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggataaaacatttcaatcctttgaacgcggtagaacgtgctaattggattttggtgagaacgcggtagaacgtacttatcacctacagttttattttgtttttctttttggtttaatctatccagcttagtaccgagtgggggaaagtgactggtgtgcctaaaaccttttctttgatactttgtaaaaatacatacagatacaatggcgaacggtaacttcaagttgtctcaattgctcaatgtggacgagatgtctgctgagcagaggagtcatttctttgacttgatgctgactaaacctgattgtgagatcgggcaaatgatgcaaagagttgttgttgataaagtcgatgacatgattagagaaagaaagactaaagatccagtgattgttcatgaagttctttctcagaaggaacagaacaagttgatggaaatttatcctgaattcaatatcgtgtttaaagacgacaaaaacatggttcatgggtttgcggctgctgagcgaaaactacaagctttattgcttttagatagagttcctgctctgcaagaggtggatgacatcggtggtcaatggtcgttttgggtaactagaggtgagaaaaggattcattcctgttgtccaaatctagatattcgggatgatcagagagaaatttctcgacagatatttcttactgctattggtgatcaagctagaagtggtaagagacagatgtcggagaatgagctgtggatgtatgaccaatttcgtgaaaatattgctgcgcctaacgcggttaggtgcaataatacatatcagggttgtacatgtaggggtttttctgatggtaagaagaaaggcgcgcagtatgcgatagctcttcacagcctgtatgacttcaagttgaaagacttgatggctactatggttgagaagaaaactaaagtggttcatgctgctatgctttttgctcctgaaagtatgttagtggacgaaggtccattaccttctgttgacggttactacatgaagaagaacgggaagatctatttcggttttgagaaagatccttccttttcttacattcatgactgggaagagtacaagaagtatctactggggaagccagtgagttaccaagggaatgtgttctacttcgaaccgtggcaggtgagaggagacacaatgcttttttcgatctacaggatagctggagttccgaggaggtctctatcatcgcaagagtactaccgaagaatatatatcagtagatgggaaaacatggttgttgtcccaattttcgatctggtcgaatcaacgcgagagttggtcaagaaagacctgtttgtagagaaacaattcatggacaagtgtttggattacatagctaggttatctgaccagcagctgaccataagcaatgttaaatcatacttgagttcaaataattgggtcttattcataaacggggcggccgtgaagaacaagcaaagtgtagattctcgagatttacagttgttggctcaaactttgctagtgaaggaacaagtggcgagacctgtcatgagggagttgcgtgaagcaattctgactgagacgaaacctatcacgtcattgactgatgtgctgggtttaatatcaagaaaactgtggaagcagtttgctaacaagatcgcagtcggcggattcgttggcatggttggtactctaattggattctatccaaagaaggtactaacctgggcgaaggacacaccaaatggtccagaactatgttacgagaactcgcacaaaaccaaggtgatagtatttctgagtgttgtgtatgccattggaggaatcacgcttatgcgtcgagacatccgagatggactggtgaaaaaactatgtgatatgtttgatatcaaacggggggcccatgtcttagacgttgagaatccgtgccgctattatgaaatcaacgatttctttagcagtctgtattcggcatctgagtccggtgagaccgttttaccagatttatccgaggtaaaagccaagtctgataagctattgcagcagaagaaagaaatcgctgacgagtttctaagtgcaaaattctctaactattctggcagttcggtgagaacttctccaccatcggtggtcggttcatctcgaagcggactgggtctgttgttggaagacagtaacgtgctgacccaagctagagttggagtttcaagaaaggtagacgatgaggagatcatggagcagtttctgagtggtcttattgacactgaagcagaaattgacgaggttgtttcagccttttcagctgaatgtgaaagaggggaaacaagcggtacaaaggtgttgtgtaaacctttaacgccaccaggatttgagaacgtgttgccagctgtcaaacctttggtcagcaaaggaaaaacggtcaaacgtgtcgattacttccaagtgatgggaggtgagagattaccaaaaaggccggttgtcagtggagacgattctgtggacgctagaagagagtttctgtactacttagatgcggagagagtcgctcaaaatgatgaaattatgtctctgtatcgtgactattcgagaggagttattcgaactggaggtcagaattacccgcacggactgggagtgtgggatgtggagatgaagaactggtgcatacgtccagtggtcactgaacatgcttatgtgttccaaccagacaaacgtatggatgattggtcgggatacttagaagtggctgtttgggaacgaggtatgttggtcaacgacttcgcggtcgaaaggatgagtgattatgtcatagtttgcgatcagacgtatctttgcaataacaggttgatcttggacaatttaagtgccctggatctaggaccagttaactgttcttttgaattagttgacggtgtacctggttgtggtaagtcgacaatgattgtcaactcagctaatccttgtgtcgatgtggttctctctactgggagagcagcaaccgacgacttgatcgagagattcgcgagcaaaggttttccatgcaaattgaaaaggagagtgaagacggttgattcttttttgatgcattgtgttgatggttctttaaccggagacgtgttgcatttcgatgaagctctcatggcccatgctggtatggtgtacttttgcgctcagatagctggtgctaaacgatgtatctgtcaaggagatcagaatcaaatttctttcaagcctagggtatctcaagttgatttgaggttttctagtctggtcggaaagtttgacattgttacagaaaaaagagaaacttacagaagtccagcagatgtggctgccgtattgaacaagtactatactggagatgtcagaacacataacgcgactgctaattcgatgacggtgaggaagattgtgtctaaagaacaggtttctttgaagcctggtgctcagtacataactttccttcagtctgagaagaaggagttggtaaatttgttggcattgaggaaagtggcagctaaagtgagtacagtacacgagtcgcaaggagagacattcaaagatgtagtcctagtcaggacgaaacctacggatgactcaatcgctagaggtcgggagtacttaatcgtggcgttgtcgcgtcacacacaatcacttgtgtatgaaactgtgaaagaggacgatgtaagcaaagagatcagggaaagtgccgcgcttacgaaggcggctttggcaagattttttgttactgagaccgtcttatgacggtttcggtctaggtttgatgtctttagacatcatgaagggccttgcgccgttccagattcaggtacgattacggacttggagatgtggtacgacgctttgtttccgggaaattcgttaagagactcaagcctagacgggtatttggtggcaacgactgattgcaatttgcgattagacaatgttacgatcaaaagtggaaactggaaagacaagtttgctgaaaaagaaacgtttctgaaaccggttattcgtactgctatgcctgacaaaaggaagactactcagttggagagtttgttagcattgcagaaaaggaaccaagcggcacccgatctacaagaaaatgtgcacgcgacagttctaatcgaagagacgatgaagaagctgaaatctgttgtctacgatgtgggaaaaattcgggctgatcctattgtcaatagagctcaaatggagagatggtggagaaatcaaagcacagcggtacaggctaaggtagtagcagatgtgagagagttacatgaaatagactattcgtcttacatgtatatgatcaaatctgacgtgaaacctaagactgatttaacaccgcaatttgaatactcagctctacagactgttgtgtatcacgagaagttgatcaactcgttgttcggtccaattttcaaagaaattaatgaacgcaagttggatgctatgcaaccacattttgtgttcaacacgagaatgacatcgagtgatttaaacgatcgagtgaagttcttaaatacggaagcggcttacgactttgttgagatagacatgtctaaattcgacaagtcggcaaatcgcttccatttacaactgcagctggagatttacaggttatttgggctagatgagtgggcggccttcctttgggaggtgtcgcacactcaaactactgtgagagatattcaaaatggtatgatggcgcatatttggtaccaacaaaagagtggagatgctgatacttataatgcaaattcagatagaacactgtgtgcactcttgtctgaattaccattggagaaagcagtcatggttacatatggaggagatgactcactgattgcgtttcctagaggaacgcagtttgttgatccgtgtccaaagttggctactaagtggaatttcgagtgcaagatttttaagtacgatgtcccaatgttttgtgggaagttcttgcttaagacgtcatcgtgttacgagttcgtgccagatccggtaaaagttctgacgaagttggggaaaaagagtataaaggatgtgcaacatttagccgagatctacatctcgctgaatgattccaatagagctcttgggaactacatggtggtatccaaactgtccgagtctgtttcagaccggtatttgtacaaaggtgattctgttcatgcgctttgtgcgctatggaagcatattaagagttttacagctctgtgtacattattccgagacgaaaacgataaggaattgaacccggctaaggttgattggaagaaggcacagagagctgtgtcaaacttttacgactggtaatatggaagacaagtcattggtcaccttgaagaagaagactttcgaagtctcaaaattctcaaatctaggggccattgaattgtttgtggacggtaggaggaagagaccgaagtattttcacagaagaagagaaactgtcctaaatcatgttggtgggaagaagagtgaacacaagttagacgtttttgaccaaagggattacaaaatgattaaatcttacgcgtttctaaagatagtaggtgtacaactagttgtaacatcacatctacctgcagatacgcctgggttcattcaaatcgatctgttggattcgagacttactgagaaaagaaagagaggaaagactattcagagattcaaagctcgagcttgcgataactgttcagttgcgcagtacaaggttgaatacagtatttccacacaggagaacgtacttgatgtctggaaggtgggttgtatttctgagggcgttccggtctgtgacggtacataccctttcagtatcgaagtgtcgctaatatgggttgctactgattcgactaggcgcctcaatgtggaagaactgaacagttcggattacattgaaggcgattttaccgatcaagaggttttcggtgagttcatgtctttgaaacaagtggagatgaagacgattgaggcgaagtacgatggtccttacagaccagctactactagacctaagtcattattgtcaggtgaagatgttaagagagcgcctaataagaaaaactcgtcttaatgcataaagaaatttattgtcaatatgacgtgtgtactcaagggttgtgtgaatgaagtcactgttcttggtcacgagacgtgtagtatcggtcatgctaacaaattgcgaaagcaagttgctgacatggttggtgtcacacgtaggtgtgcggaaaataattgtggatggtttgtctgtgttgttatcaatgattttacttttgatgtgtataattgttgtggccgtagtcaccttgaaaagtgtcgtaaacgtgttgaaacaagaaatcgagaaatttggaaacaaattcgacgaaatcaagctgaaaacatgtctgcgacagctaaaaagtctcataattcgaagacctctaagaagaaattcaaagaggacagagaatttgggacaccaaaaagatttttaagagatgatgttcctttcgggattgatcgtttgtttgctttttgattttattttatattgttatctgtttctgtgtatagactgtttgagattggcgcttggccgactcattgtcttaccataggggaacggactttgtttgtgttgttattttatttgtattttattaaaattctcaatgatctgaaaaggcctcgaggctaagagattattggggggtgagtaagtacttttaaagtgatgatggttacaaaggcaaaaggggtaaaacccctcgcctacgtaagcgttattacgcccgtctgtacttatatcagtacactgacgagtccctaaaggacgaaacgggcccctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatatcaactcccctatccattgctcaccgaatggtacaggtcggggacccgaagttccgactgtcggcctgatgcatccccggctgatcgaccccagatctggggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgtcttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagtttgggccctatatatggatccaattgcaatgatcatcatgacagatctgcgcgcgatcgatatcagcgctttaaatttgc

SEQ ID NO: 6 - DNA, 15232 bp, synthetic construct
  source 1..15232: mol_type = other DNA
taaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgttggtagtagcgactccatggttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataagcttgcatgcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgacctcaacacaacatatacaaaacaaacgaatctcaagcaatcaagcattctacttctattgcagcaatttaaatcatttcttttaaagcaaaagcaattttctgaaaattttcaccatttacgaacgatactcgagtaatctagatggattacaaggaccacgacggggattacaaggaccacgacattgattacaaggatgatgatgacaagatggctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgagctcagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatctgtcgatcgaccatggggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggatcgatcctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaa

SEQ ID NO: 7 - DNA, 26 bp, synthetic construct
  source 1..26: mol_type = other DNA
ggtaattcagcttatctttggagctc

SEQ ID NO: 8 - DNA, 26 bp, synthetic construct
  source 1..26: mol_type = other DNA
aaaacaaacctttaaaggattaaagt

SEQ ID NO: 9 - DNA, 26 bp, synthetic construct
  source 1..26: mol_type = other DNA
tcacttcttcttcttcgcctgccccg

SEQ ID NO: 10 - DNA, 26 bp, synthetic construct
  source 1..26: mol_type = other DNA
tttccagttctacaaggttcgggaga

SEQ ID NO: 11 - DNA, 26 bp, synthetic construct
  source 1..26: mol_type = other DNA
tcgtacaggagcgagtgcttagggag

SEQ ID NO: 12 - DNA, 26 bp, synthetic construct
  source 1..26: mol_type = other DNA
atggattacaaggaccacgacgggga

SEQ ID NO: 13 - DNA, 26 bp, synthetic construct
  source 1..26: mol_type = other DNA
ataaaacattgcacctatggtgttgc

SEQ ID NO: 14 - DNA, 26 bp, synthetic construct
  source 1..26: mol_type = other DNA
ttactcacagaatctaagtccactcg

SEQ ID NO: 15 - DNA, 26 bp, synthetic construct
  source 1..26: mol_type = other DNA
atgtcccgaagacattaaactacggt

SEQ ID NO: 16 - DNA, 26 bp, synthetic construct
  source 1..26: mol_type = other DNA
gggcgtaataacgcttacgtaggcga

SEQ ID NO: 17 - DNA, 23 bp, synthetic construct
  source 1..23: mol_type = other DNA
caccgactcggtgccactttttc

SEQ ID NO: 18 - DNA, 26 bp, synthetic construct
  source 1..26: mol_type = other DNA
cagcttacattttcttgaaccgtagc

SEQ ID NO: 19 - DNA, 26 bp, synthetic construct
  source 1..26: mol_type = other DNA
atggcgaacggtaacttcaagttgtc

SEQ ID NO: 20 - DNA, 26 bp, synthetic construct
  source 1..26: mol_type = other DNA
tgtctcgacgcataagcgtgattcct

SEQ ID NO: 21 - DNA, 26 bp, synthetic construct
  source 1..26: mol_type = other DNA
atggaagacaagtcattggtcacctt

SEQ ID NO: 22 - DNA, 26 bp, synthetic construct
  source 1..26: mol_type = other DNA
ttaagacgagtttttcttattaggcg

SEQ ID NO: 23 - DNA, 20 bp, synthetic construct
  source 1..20: mol_type = other DNA
tggacgaagagcatcagggg

SEQ ID NO: 24 - DNA, 26 bp, synthetic construct
  source 1..26: mol_type = other DNA
tataagggcccgtactgaattaacgc

SEQ ID NO: 25 - DNA, 24 bp, synthetic construct
  source 1..24: mol_type = other DNA
attgttggtagtagcgactccatg

SEQ ID NO: 26 - DNA, 24 bp, synthetic construct
  source 1..24: mol_type = other DNA
aaaccatggagtcgctactaccaa

SEQ ID NO: 27 - DNA, 26 bp, synthetic construct
  source 1..26: mol_type = other DNA
taggcgggcccggtttgcgtattggc

SEQ ID NO: 28 - DNA, 29 bp, synthetic construct
  source 1..29: mol_type = other DNA
tatatacctgcagggtactgaattaacgc

SEQ ID NO: 29 - DNA, 23 bp, synthetic construct
  source 1..23: mol_type = other DNA
taacgccgaattaattcctaggc

SEQ ID NO: 30 - DNA, 37 bp, synthetic construct
  source 1..37: mol_type = other DNA
atatgtttaaacacactgatagtttaattcccgatct

SEQ ID NO: 31 - DNA, 31 bp, synthetic construct
  source 1..31: mol_type = other DNA
gaaactcgagatggattacaaggaccacgac

SEQ ID NO: 32 - DNA, 35 bp, synthetic construct
  source 1..35: mol_type = other DNA
atatttaagctttcacttcttcttcttcgcctgcc

SEQ ID NO: 33 - DNA, 20 bp, synthetic construct
  source 1..20: mol_type = other DNA
ctgagcgcaaaagtacacca

SEQ ID NO: 34 - DNA, 31 bp, synthetic construct
  source 1..31: mol_type = other DNA
atataccatggatggcgaacggtaacttcaa

SEQ ID NO: 35 - DNA, 20 bp, synthetic construct
  source 1..20: mol_type = other DNA
ttgggtggaatcagtttcgt

SEQ ID NO: 36 - DNA, 20 bp, synthetic construct
  source 1..20: mol_type = other DNA
tcttccaaagtcgagccagt

SEQ ID NO: 37 - protein, 1423 aa, Streptococcus pyogenes
MDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 38 - RNA, 20 nt, synthetic construct
  source 1..20: mol_type = other RNA
tttggtagtagcgactccat

SEQ ID NO: 39 - protein, 1368 aa, Staphylococcus aureus [sequence omitted]

SEQ ID NO: 40 - protein, 763 aa, Biggievirus Mos11
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTSHHHHHH

SEQ ID NO: 41 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 42 - protein, 204 aa, Tobacco rattle virus
  source 1..204: note = Tobacco rattle virus 2
MGDMYDESFDKSGGPADLMDDSWVESVSWKDLLKKLHSIKFALQSGRDEITGLLAALNRQCPYSPYEQFPDKKVYFLLDSRANSALGVIQNASAFKRRADEKNAVAGVTNIPANPNTTVTTNQGSTTTTKANTGSTLEEDLYTYYKFDDASTAFHKSLTSLENMELKSYYRRNFEKVFGIKFGGAAASSSAPPPASGGPIRPNP

SEQ ID NO: 43 - protein, 252 aa, Tobacco rattle virus
  source 1..252: note = Tobacco rattle virus 1
MEDKSLVTLKKKTFEVSKFSNLGAIELFVDSRRKRPKYFHRRRETVLNHVGGKKSEHKLDVFDQRDYKMIKXYTFLKIVGVQLVVTSHLPADTPGFIQIDLLDSRLTEKRKKGKTIQRFKARACDNCSVAQYKVEYSISTQENVLDVWKVGCISEGVPVCDGTYPFSIEVSLIWVATDSTRRLNVEELNSSDYIEGDFTDQEVFGEFMSLKQVEMKTIEPKYDGPYRPATTRPKSLLSSEDVKRASNKKNSS

SEQ ID NO: 44 - protein, 1707 aa, Tobacco rattle virus
  source 1..1707: note = Tobacco rattle virus 1
MANGNFKLSQLLNVDEMSAEQRSHFFDLMLTKPDCEIGQMMQRVVVDKVDDMIRERKTKDPVIVHEVLSQKEQNKLMEIYPEFNIVFKDDKNMVHGFAAAERKLQALLLLDRVPALQEVDDIGGQWSFWVTRGEKRIHSCCPNLDIRDDQREISRQIFLTAIGDQARSGKRQMSENELWMYDQFRKNIAAPNAVRCNNTYHGCTCRGFSDGKKKGAQYAIALHSLYDFKLKDLMATMVEKKTKVGHAAMLFAPESMLVDEGPLPSVDGYYMKKNGKIYFGFEKDPSFSYIHDWEEYKKYLLGKPVSYQGNVFYFEPWQVRGDTMLFSIYRIAGVPRRSLSSQEYYRRIYISRWENMVVVPIFDLVESTRELVKKDLFVEKQFMDKCLDYIARLSDQQLTISNVKSYLSSNNWVLFINGAAVKNKQSVDSRDLQLLAQTLLVKEQVARPVMRELREAILTETKPITSLTDVLGLISRKMWKQFANKIAVGGFVGMVGTLIGFYPKKVLTWAKDTPNGPELCYENSHKTKVIVFLSVVYAIGGITLMRRDIRDGLVKKLCDMFDIKRGAHVLDVENPCRYYDINDFFSSLYSASESGETVLPDLSEVKAKSDKLLQQKKEIADEFLSAKFSNYSGSSVRTSPPSVVGSSRSGLGLLLEDSNVLTQARVGVSRKVADEEIMEQFLSGLIDTEAEIDEVVPAFSAECERGETSGTKVLCNLLTPPGFENVLPAVKPLVSKGKTVKRVDYFQVMGGERLPKRPVVSGDDSVDARREFLYYLDAERVAQNDEIMSLYRDYSRGVIRTGGQNYPHGLGVWDVEMKNWCIRPVVTEHAYVSNPDKRMDDWSGYLEVAVWERGMLVNDFAVERMSDYVIVCDQTYLCNNRLILDNLSALDLGPVNCSFELVDGVPGCGKSTMIVNSANPCVDVVLSTGRAATDDLIERFASKGFPCKLKRRVKTVDSFLMHCVDGSLTGDVLHFDEALMAHAGMVYFCAQIAGAKRCICQGDQNQISFKPRVSQVDLRFSSLVGKFDIVTEKRETYRSPADVAAVLNKYYTGDVRTHNATANSMTVRKIVSKEQVSLKPGAQYITFLQSEKKELVNLLALRKVAAKVSTVHESQGETFKDVVLVRTKPTDDSIARGREYLIVALSRHTQSLVYETVKEDDVSKEIRESAALTKAALARFFVTETVLXRFRSRFDVFRHHEGPCAVPDSGTITDLEMWYDALFPGNSLRDSSLDGYLVATTDCNLRLDNVTIKSGNWKDKFAEKETFLKPVIRTAMPDKRKTTQLESLLALQKRNQAAPDLQENVHATVLIEETMKKLKSVVYDVGKIRADPIVNRAQMERWWRNQSTAVQAKVVADVRELHEIDYSSYMFMIKSDVKPKTDLTPQFEYSALQTVVYHEKLINSLFGPIFKEINERKLDAMQPHFVFNTRMTSSDLNDRVKFLNTEAAYDFVEIDMSKFDKSANRFHLQLQLEIYRLFGLDEWAAFLWEVSHTQTTVRDIQNGMMAHIWYQQKSGDADTYNANSDRTLCALLSELPLEKAVMVTYGGDDSLIAFPRGTQFVDPCPKLATKWNFECKIFKYDVPMFCGKFLLKTSSCYEFVPDPVKVLTKLGKKSIKDVQHLAEIYISLNDSNRALGNYMVVSKLSESVSDRYLYKGDSVHALCALWKHIKSFTALCTLFRDENDKELNPAKVDWKKAQRAVSNFYDW

SEQ ID NO: 45 - protein, 141 aa, Tobacco rattle virus
  source 1..141: note = Tobacco rattle virus 1
MTCVLKGCVNEVTVLGHETCSIGHANKLRKQVADMVGVTRRCAENNCGWFVCVVINDFTFDVYNCCGRSHLEKCRKRVETRNREIWKQIRRNQAENMSATAKKSHNSKTSKKKFKEDREFGTPKRFLRDDVPFGIDRLFAF

SEQ ID NO: 46 - protein, 292 aa, Tobacco rattle virus
  source 1..292: note = Tobacco rattle virus 2
MVIVTKGAYVHEFPRTAEWNAFAQILSREHGYIVSDTALEGAAKPYYVVNSSGFYGPPGLDGLISTLDRELQYYSKLLYEIKGLGVMSDENVFGTQYDGNLTARVSRLERRLNPMSNIGSSSRPWSEHKSAVKKADLERYVYANFADWSNHLGPAGKSTREVVKYLMYRMGYYSDTSGIGHDLNYKHFRDHLDIYNLTCSPPFLVSNAVVDGHYARDKFVSFQGVCGFNPMFPDVNGLKSSWSLGRQLDDIRSQKKEVSGTNQEPNYYYDGDTLKPIGSGASVVGERRPGWR